(19) United States
(12) Patent Application Publication
(10) Pub. No.: US 2010/0029613 A1
(43) Pub. Date: Feb. 4, 2010

(54) TREATMENT AND PREVENTION OF EPILEPSY

(75) Inventors: Maiken Nedergaard, Webster, NY (US); Guo Feng Tian, Rochester, NY (US); Takahiro Takano, Rochester, NY (US)

Correspondence Address: NIXON PEABODY LLP - PATENT GROUP, 1100 CLINTON SQUARE, ROCHESTER, NY, 14604, US

(73) Assignee: University of Rochester, Rochester, NY (US)

(21) Appl. No.: 11/719,238

(22) PCT Filed: Nov. 14, 2005
(86) PCT No.: PCT/US05/41058

§ 371 Date: Aug. 22, 2008

Related U.S. Application Data
(60) Provisional application No. 60/627,847, filed on Nov. 15, 2004.

(51) Int. Cl. A61K 31/55; C12Q 1/02; C12N 5/02

(52) U.S. Cl. 514/212.01; 435/29; 435/375

(57) ABSTRACT

The present invention is directed to a method of treating or preventing epileptic seizures in a subject and a method of inhibiting hypersynchronous burst activity of neurons by administering an agent which interferes with glutamate, aspartate, and/or ATP release from astrocytes. Also presented is a method of identifying agents suitable for treating or preventing epileptic seizures.

This application claims the benefit of U.S. Provisional Patent Application Ser. No. 60/627,847, filed Nov. 15, 2004, which is hereby incorporated by reference in its entirety.

The subject matter of this application was made with support from the National Institute of Health under Grant No. 5-28926. The U.S. Government may have certain rights.

### FIELD OF THE INVENTION

The present invention is directed to the treatment and prevention of epilepsy.

### BACKGROUND OF THE INVENTION

Epilepsy is a neurological disorder in which normal brain function is disrupted as a consequence of intensive burst activity from groups of neurons (Wyllie, E., “The Treatment of Epilepsy Principles and Practice,” (Lippincot, Williams, and Wilkins, New York (2001)). Epilepsies result from long-lasting plastic changes in the brain affecting the expression of receptors and channels, and involve sprouting and reorganization of synapses, as well as reactive gliosis (Heinemann et al., “Contribution of Astrocytes to Seizure Activity,” Adv. Neurol. 79:583-590 (1999); Rogawski et al., “The Neurobiology of Antiepileptic Drugs,” Nat. Rev. Neurosci. 5:553-564 (2004)). Epileptic seizures can result from a primary epileptic disorder, such as Rolandic epilepsy, Lennox Gastaut or West syndrome, and juvenile myoclonic epilepsies, petit mal, or idiopathic temporal lobe seizures, psychomotor epilepsy or mesial temporal sclerosis. Epileptic seizures can also result from pediatric or adult-onset hereditary metabolic disorders or as a manifestation or late sequela to stroke, traumatic brain injury, intracerebral hemorrhage, tumors, infection, vascular malformation, metabolic, endocrine or electrolyte disturbance, and coagulation dysfunction. Several lines of evidence suggest a key role of glutamate in the pathogenesis of epilepsy. Local or systemic administration of glutamate agonists triggers excessive neuronal firing, whereas glutamate receptor (GluR) antagonists have anticonvulsant properties (Meldrum, B. S., “Update on the Mechanism of Action of Antiepileptic Drugs,” Epilepsia 37 (Suppl.):6, S4-11 (1996)).

Paroxysmal depolarization shifts (PDSs) are abnormal prolonged depolarizations with repetitive spiking and are reflected as interictal discharges in the electroencephalogram (Heinemann et al., “Contribution of Astrocytes to Seizure Activity,” Adv. Neurol. 79:583-590 (1999); Rogawski et al., “The Neurobiology of Antiepileptic Drugs,” Nat. Rev. Neurosci. 5:553-564 (2004)).

Astrogliosis is a prominent feature of the epileptic brain, with autopsy and surgical resection specimens demonstrating that post-traumatic seizures and chronic temporal lobes epilepsy, may originate from gliotic scars (Tashiro et al., “Calcium Oscillations in Neocortical Astrocytes under Epileptiform Conditions,” J. Neurobiol. 50:45-55 (2002); Rothstein et al., “Knockout of Glutamate Transporters Reveals a Major Role for Astroglial Transport in Excitotoxicity and Clearance of Glutamate,” Neuron 16:675-686 (1996); Duffy et al., “Modulation of Neuronal Excitability by Astrocytes,” in Jasper's Basic Mechanisms of Epilepsies, Third Edition: Advances in Neurology, Vol 79, Delgado-Escueta et al., eds., Lippincott Williams & Wilkins, Philadelphia (1999)). In addition, astrocytes can modulate synaptic transmission through release of glutamate (Haydon, P. G., “GLIA: Listening and Talking to the Synapse,” Nat. Rev. Neurosci. 2:185-193 (2001)). For example, spontaneous astrocytic Ca²⁺ oscillations drive NMDA-receptor-mediated neuronal excitation in the rat ventrobasal thalamus and activate groups of neurons in hippocampus (Fellin et al., “Neuronal Synchrony Mediated by Astrocytic Glutamate Through Activation of Extrasynaptic NMDA Receptors,” Neuron 43:729-743 (2004); Angulo et al., “Glutamate Released from Glial Cells Synchronizes Neuronal Activity in the Hippocampus,” J. Neurosci. 24:6920-6927 (2004)). These and other studies have pointed to glutamate as a key transmitter of bi-directional communication between astrocytes and neurons (Nedergaard et al., “Beyond the Role of Glutamate as a Neurotransmitter,” Nat. Rev. Neurosci. 3:748-755 (2002); Haydon, P. G., “GLIA: Listening and Talking to the Synapse,” Nat. Rev. Neurosci. 2:185-193 (2001)). Nonetheless, experimental observations implicating astrocytes in initiation, maintenance, or spread of seizure activity, have not existed until now.

The present invention is directed to overcoming these and other deficiencies in the art.

### SUMMARY OF THE INVENTION

A first aspect of the present invention relates to a method of treating or preventing epileptic seizures in a subject. The method involves administering an agent which interferes with glutamate, aspartate, and/or ATP release from astrocytes to the subject under conditions effective to treat or prevent epileptic seizures.

Another aspect of the present invention relates to a method of inhibiting hypersynchronous burst activity of a large group of neurons. The method involves administering an agent which interferes with glutamate, aspartate, and/or ATP release from astrocytes to the group of neurons under conditions effective to inhibit hypersynchronous burst activity.

A further aspect of the present invention relates to a method of identifying agents suitable for treating or preventing epileptic seizures. The method involves contacting astrocytes with one or more candidate compounds, evaluating the astrocytes for glutamate, aspartate, and/or ATP release, and then identifying the candidate compounds which interfere with glutamate, aspartate, and/or ATP release as agents potentially suitable for treating or preventing epileptic seizures.

According to the present invention, glutamate released by astrocytes can trigger PDSs in several models of experimental seizure. A unifying feature of seizure activity was its consistent association with antecedent astrocytic Ca²⁺ signaling. Oscillatory, tetrodotoxin (TTX)-insensitive increases in astrocytic Ca²⁺ preceded or occurred concomitantly with PDSs, and targeting astrocytes by photolysis of caged Ca²⁺ evoked PDSs. Furthermore, several anti-epileptic agents, including valproate, gabapentin, and phenyloin, potently reduced astrocytic Ca²⁺ signaling detected by 2-photon imaging in live animals. This suggests that pathologic activation of astrocytes likely play a central role in the genesis of epilepsy, as well as in the pathways targeted by current anti-epileptics. The observation that astrocytes release glutamate via a regulated Ca²⁺ dependent mechanism (Parpura et al., “Glutamate-Mediated Astrocyte-Neuron Signalling,” Nature 369:744-747 (1994); Bezzi et al., “Prostaglandins Stimulate Calcium-Dependent Glutamate Release in Astrocytes,” Nature 391:281-285 (1998); Fellin et al., “Neuronal Synchrony Mediated by Astrocytic Glutamate Through Activation of Extrasynaptic NMDA Receptors,” Neuron 43:729-743 (2004); Angulo et al., “Glutamate Released from Glial Cells Synchronizes Neuronal Activity in the Hippocampus,” J. Neurosci. 24:6920-6927 (2004), which are hereby incorporated by reference in their entirety) leads one to hypothesize that glutamate released by astrocytes plays a causal role in synchronous firing of large populations of neurons.

### BRIEF DESCRIPTION OF THE DRAWINGS

FIGS. 1A-F illustrate that synaptic activity is not required for PDSs in hippocampal slices evoked by 4-AP. FIG. 1A shows whole-cell recording of CA1 pyramidal neuron during epileptiform activity triggered by 4-AP (100 μM, upper trace) combined with field potential recording (lower trace). Spontaneous neuronal depolarization events elicit trains of action potentials, which are mirrored by negative deflections of the field potential. FIG. 1B shows that the addition of TTX (1 μM) eliminated neuronal firing, but not the transient episodes of neuronal depolarizations and the drop in field potential. FIG. 1C shows 4-AP induced PDSs in a CA1 pyramidal neuron. FIG. 1D shows that this effect continues in the presence of a cocktail of voltage-gated Ca²⁺ blockers, Nifedipine (L-type channel blocker, 10 μM), Mibefradil (T-type channel blocker, 10 μM), Omega-Conotoxin MVIIC (P/Q type Blocker, 1 μM), Omega-Conotoxin GVIA (N-type blocker, 1 μM), SNX-482 (R-type blocker, 0.1 μM) and TTX (1 μM). FIGS. 1E-F show an astrocytic membrane potential decline of 0.5-1.0 mV during PDSs before, and after addition of TTX, respectively. In all recordings, the field potential electrode was placed less than 30 μm from either the neuronal (FIGS. 1A-D), or astrocytic cell body (FIGS. E-F).

FIGS. 2A-I show that PDSs are mediated by release of glutamate from action potential-independent sources. FIG. 2A depicts representative traces of field potential recording in 4-AP; 4-AP and TTX; 4-AP, TTX, 2-amino-5-phosphonovalerate (APV) (50 μM), and 6-cyano-7-nitroquinoxaline-2,3-dione (CNQX) (20 μM). FIG. 2B shows frequency, amplitude and area (amplitude x duration) plotted as a function of time (n=7). Spontaneous field potential events were observed in all slices exposed to 4-AP. The frequency and amplitude of PDSs were reduced by 20-35% by TTX and by 85-90% by APV and CNQX. FIG. 2C shows normalized mean values of frequency, amplitude, and area (amplitude x duration) during exposure to 4-AP; 4-AP+TTX; and 4-AP+TTX+APV/CNQX, during washout of APV/CNQX (4-AP+TTX), and during washout of TTX (4-AP) (n=7). FIG. 2D shows that the cocktail of VGCC blockers (Nifedipine, Mibefradil, Omega-Conotoxin MVIIC, Omega-Conotoxin GVIA, SNX-482, same concentration as in FIG. 1) and TTX did not decrease the frequency or amplitude of PDSs compared with TTX alone (n=5). FIG. 2E shows that D,L-threo-beta-benzyloxyaspartate (TBOA, a glutamate transport inhibitor, 100 μM) did not reduce the occurrence of PDSs, but increased the frequency, amplitude and area of PDSs significantly suggesting that inverted transport of glutamate did not contribute to PDSs (n=6). FIG. 2F shows that (S)-Alpha-methyl-4-carboxy-phenylglycine ((S)-MCPG, a non-selective mGluR antagonist, 1 mM) did not decrease the frequency or amplitude of PDSs compared with TTX alone (n=7). FIG. 2G shows that CNQX alone significantly reduced PDSs (n=6). FIG. 2H shows that APV alone highly significantly reduced PDSs (n=6). FIG. 2I shows that TTX added before (10-15 min) had no effect on frequency of PDSs, but significantly reduced the amplitude of PDSs compared with slices first exposed to TTX 20 min after addition of 4-AP (n=7). * P<0.05; ** P<0.001; Student's t-test; mean±s.d.

FIGS. 3A-D show spontaneous depolarization shifts in four experimental models of epilepsy. In FIG. 3A, hippocampal slices were perfused with Mg²⁺-free solutions. Traces in left panel are representative field potential recordings in: Mg²⁺-free solution (upper); after addition of 1 μM TTX (middle), and after addition of TTX+50 μM APV and 20 μM CNQX (lower). Plots of the frequency, amplitude, and area (amplitude x duration) of PDSs are also shown. FIGS. 3B-D show similar sets of observations in hippocampal slices exposed to bicuculline (FIG. 3B, 30 μM), penicillin (FIG. 3C, 2000 U/ml), and Ca²⁺-free solution (FIG. 3D, 1 mM EGTA). * P<0.05; ** P<0.001; Student's t-test; mean±s.d.; n=5-7.

FIGS. 4A-E show that epileptogenic agents evoke oscillatory increases in astrocytic cytosolic Ca²⁺ concentration, which precedes PDSs, and PDSs are spatially confined to small domains. FIG. 4A, upper panel shows 2-photon imaging of astrocytic Ca²⁺ oscillations in stratum radiatum of the CA1 region in hippocampal slices exposed to 4-AP (100 μM) and TTX (1 μM). The frames were acquired with an interval of 8.2 s following 20 min exposure to 4-AP and TTX. White arrows indicate astrocytes with oscillatory increases in Ca²⁺. Scale bar, 50 μm. FIG. 4A, lower panel is a histogram showing the frequency of Ca²⁺ oscillations in hippocampal slices exposed to 4-AP (100 μM), Mg²⁺-free solution, bicuculline (30 μM), penicillin (2000 U/ml), and Ca²⁺-free solution with and without TTX (1 μM) (mean±s.d., n=7). FIG. 4B shows average increases in cytosolic Ca²⁺ in cultured astrocytes in response to 4-AP, Mg²⁺-free solution, bicuculline, penicillin, and Ca²⁺-free solution (mean±s.d., n=3). * P<0.05; ANOVA with Dunnett compared with vehicle. FIG. 4C, upper panel, shows 2-photon imaging of Ca²⁺ signaling combined with the field recordings in hippocampal slices exposed to 4-AP. The pipette solution contained 1 μM fluorescein-dextran to make the electrode visible during imaging (red in pseudocolor). White arrow indicates an astrocyte with a transient increase in cytosolic Ca²⁺. Scale bar, 30 μm. FIG. 4C, middle panel shows the rise in astrocytic Ca²⁺ concentration (upper tracing) preceded the negative deflection of the field potential (lower tracing). Numbers on the Ca²⁺ trace represent images in the upper panel. FIG. 4C, lower panel shows a histogram mapping the latency between the onset of oscillatory increases in Ca²⁺ with the onset of drop in field potential. In all cases, astrocytic Ca²⁺ increment preceded the depolarization shift. In FIG. 4D, both electrodes were placed in stratum radiatum of CA1. Representative tracings and summary histograms of dual field potential recordings with the electrodes placed at a distance of less than 100 μM (left panel); 100-200 μm (middle panel); and greater than 200 μm apart (right panel) are shown. In FIG. 4E, one electrode of the paired recordings was placed in stratum pyramidale of CA1 and the other one in stratum radiatum with a distance of less than 100 μM. The left panel shows representative tracings (top) and summary histograms (bottom) of dual field potential recordings. The central panel shows expanding recording traces (top) within the shadow area in the top of the left panel, the rise in astrocytic Ca²⁺ concentration (bottom) preceded the negative deflections of the field potentials. The numbers and letters are indicated in the right panel. In the right panel, the top photo is a DIC image which indicates the locations of the two electrodes. The other three photos are the 2-photon images of Ca²⁺ signaling in hippocampal slice exposed to 4-AP. White arrows indicate astrocytes with transient increases in cytosolic Ca²⁺. Scale bar, 20 μm.

FIGS. 5A-C show that astrocytes are the primary source of glutamate in experimental seizure. FIG. 5A shows that photolysis of caged Ca²⁺ (NP-EGTA) in an astrocyte elicits a local depolarization shift in the presence of 1 μM TTX. The upper panel shows a sequence of pseudocolor images of an astrocyte loaded with NP-EGTA/AM and fluo-4/AM. Delivery of UV pulses targeting the astrocyte elevates cytosolic Ca²⁺ and triggers a spontaneous depolarization shift with a latency of 1.3 s. Scale bar, 10 μm. The lower panel shows traces of astrocytic Ca²⁺ concentration and field potential. Black arrow represents the delivery of UV pulses. Numbers on the Ca²⁺ trace represent images in the upper panel. FIG. 5B shows a profile of amino acids released in an adult rat perfused with 4-AP (5 mM) and TTX (10 μM) through a microdialysis probe implanted in hippocampus. The histogram maps amino acid release before and after stimulation. * P<0.05; ** P<0.001; paired Student's test, mean±s.d., n=4. FIG. 5C shows that anion channel inhibitors, both 5-nitro-2-(3-phenylpropylamino) benzoic acid (NPPB, 100 μM) and flufenamic acid (FFA, 100 μM), which reduce glutamate release from astrocytes, markedly decreased the frequency, amplitude and area of PDSs. ** P<0.001 (compared with 4-AP groups by paired Student's t-test), mean±s.d., n=7.

FIGS. 6A-H show experimental seizure in adult mice and the effect of anti-epileptic agents on astrocytic Ca²⁺ signaling. In FIG. 6A, the primary somatosensory cortex was exposed and loaded with fluo-4/AM and the astrocyte specific dye, sulforhodamine (SR101). Spacebar indicates 25 μm. FIG. 6B shows normal EEG activity and stable astrocytic cytosolic Ca²⁺ levels under resting condition in an anesthetized mouse. Images were collected 130 μm below the pial surface. In FIG. 6C, 4-AP was delivered locally by an electrode and triggered delayed spontaneous episodes of high frequency, large amplitude discharges and astrocytic Ca²⁺ signaling. FIG. 6D shows that in an animal receiving valproate (450 mg/kg i.p.), 4-AP induced seizure activity and astrocytic Ca²⁺ signaling were reduced. In FIG. 6E, astrocytic Ca²⁺ wave induced by iontophoretic application of ATP during basal condition is shown, and in FIG. 6F, in an animal additionally treated with valpropate (450 mg/kg i.p.). Lower panels map changes in fluo-4 emission (ΔF/F) as a function of time. FIG. 6G is a histogram summarizing the effect of valproate, gabapentin (200 mg/kg i.p.), and phenyloin (100 mg/kg i.p.) on 4-AP induced astrocytic Ca²⁺ signaling (5-30 min after delivery of 4-AP). FIG. 6H is a histogram summarizing the effect of valproate (450 mg/kg i.p.), gabapentin (200 mg/kg i.p.), and phenyloin (100 mg/kg i.p.) on ATP-induced Ca²⁺ waves. * P<0.05; ** P<0.001; Student's t-test; mean±s.d.; n=5-7. Space bar indicates 50 μm.

FIGS. 7A-B depict an intracortical ferric chloride injection model of epilepsy. In FIG. 7A, a paroxysmal depolarization shift (arrow) preceded epileptiform bursting activities in a mouse, which received an intracortical injection of ferric chloride 6 months prior. The upper trace shows an EEG recording in AC (1-100 Hz) mode while the lower trace shows an EEG recording in DC (0-1000 Hz) mode. FIG. 7B shows summary histograms of amplitude of PDSs, frequency, duration, and amplitude of seizure activity in mice (n=13) with intracortical injection of ferric chloride 6 months prior. Mean±SD is depicted.

FIGS. 8A-B depict a genetic model in epilepsy. In FIG. 8A, a paroxysmal depolarization shift (black arrow) preceded epileptiform bursting activities in a 2-month-old genetic (B6.D2-Cacnalatg/J, JAX#000544) epilepsy mouse. A paroxysmal depolarization shift (arrow) preceded epileptiform bursting activities in a 2-month-old genetic epilepsy mouse. The upper trace shows an EEG recording in AC (1-100 Hz) mode while the lower trace shows an EEG recording in DC (0-1000 Hz) mode. FIG. 8B shows summary histograms of amplitude of PDSs, frequency, duration, and amplitude of seizure activity in 2-month-old genetic epilepsy mice (n=4). Mean±SD is depicted.

FIGS. 9A-B show that GFAP and Cx43 expression is upregulated in an intracortical ferric chloride injection model of epilepsy. FIG. 9A shows GFAP (green) and Cx43 (red) expressions in cortex of an age-matched control mouse. Cx43 immunoreactive plaques (some indicated by white arrows) are small and evenly distributed. FIG. 9B shows GFAP (green) and Cx43 (red) expressions in cortex of a mouse, which received an intracortical injection of ferric chloride 2 months prior. Reactive gliosis and a massive increase in the size of Cx43 (some indicated by white arrowheads) is evident (same mouse as in FIG. 7A). Scale bar indicates 10 μm.

FIGS. 10A-C show astrocytic Ca²⁺ increases are associated with a transient increase in cell volume. FIG. 10A indicates exposure to ATP (100 μM induces swelling of cultured astrocytes. Confocal vertical cross-sectional images of confluent astrocyte cultures with ≈3-5 cells in the field of view loaded with calcein/AM (5 μM for 30 min) were constructed from repetitive x-z line scans at 488 nm excitation. Two images of cross-sectional area before (red) and 1 min after the exposure to ATP (green) are overlapped to indicate the change in cell volume. Over-lapped areas (no change before and after ATP exposure) are displayed as white. Hypotonicity (214 mOsM) also induced cellular swelling. FIG. 10B shows quantification of relative changes in cross-sectional areas 1 min after addition of vehicle (control, n=12); ATP (100 μM, n=23); ATP to cultures preloaded with BAPTA (20 μM for 30 min, n=11); UTP (100 μM, n=15) and hypotonicity (214 mOsM, n=6). *, P<0.01 compared with control, Tukey-Kramer test. FIG. 10C shows Coulter counter analysis of relative changes in astrocytic cell volume evoked by ATP. ATP exposure of astrocytes in suspension triggered a transient increase in cell volume at 30 and 60 sec. FIG. 10C inset shows hypotonicity induced a large and sustained increase in astrocytic cell volume. n=5; *, P<0.05 compared with control, t test. mean±SEM.

FIGS. 11A-E show pharmacology of Ca²⁺-dependent glutamate release from astrocytes. FIG. 11A shows ATP-induced glutamate release from astrocytic cultures detected by fluorescence enzymatic assay. BAPTA/AM (20 μM for 30 min) and the anion channel blocker NPPB (100 μM) attenuated the glutamate efflux. FIG. 1B, upper panel shows a comparison of ATP-induced and hypotonic-induced glutamate release. ATP-induced release was inhibited by BAPTA/AM (20 μM for 30 min) and thapsigargin (1 μm). Anion channel blockers, NPPB (100 μM), FFA (100 μM), and gossypol (10 μM) all eliminated ATP-induced glutamate release, whereas removal of Ca²⁺ had no effect. A glutamate transport blocker, DL-threo-β-benzyloxyaspartic acid (TBOA) (100 μM), had no effect. Similarly, the inhibition of vesicular release by bafilomycin A1 (1 μM for 1 h) or tetanus neurotoxin (TeNT; 2 μg/ml for 24 h) had no effect. A glutamine synthetase inhibitor, methionin sulfoximine (MSO; 1.5 mM for 2 h), increased ATP-induced glutamate releases. (n=5; *, P<0.01 compared with control, Tukey-Kramer test). The release from cultured astrocytes from Cx43 KO mice was not significantly different from the release from matched wild-type littermates (n=4; P=0.64, t test). FIG. 11B lower panel shows hypoosmotic stimulation (214 mOsM) induced glutamate release that was Ca²⁺-independent, but otherwise had the same pharmacological profile as ATP-induced release (n=5, *, P<0.01 compared with control, Tukey-Kramer test). FIG. 11C shows glutamate release was mediated by P2YR activation. UTP (100 μM; a P2Y agonist) induced glutamate release with a potency comparable to that of ATP. By contrast, αβ-meATP (100 μM), (a P2X agonist), and Bz-ATP (100 μM) elicited little glutamate release. Similarly, Ox-ATP (300 μM) for 1 h) did not significantly attenuate the release. Reactive Blue 2 (RB2, 30 μM; a P2Y antagonist) blocked the release. A cycloloxygenase inhibitor indomethacin (10 μM) also failed to inhibit ATP-induced glutamate release. n=4; *, P<0.01 compared with ATP, Tukey-Kramer test. FIG. 11D, left panel, shows dose-response curve of ATP-induced glutamate release (n=3). FIG. 11D, right panel, shows glutamate release by 10 μM ATP is smaller than the release by 100 μM ATP but retains sensitivity to NPPB (100 μM) and TeNT (10 μg/ml overnight) (n=3≈5). *, P<0.01, Tukey-Kramer test, mean±SEM. FIG. 11E shows cell swelling is required for ATP-induced glutamate release. ATP (100 μM) was added at the time as the osmolarity change, which was accomplished by adding sucrose (for hypertonicity) or distilled water (for hypotonicity). Hyperosmolality >15% completely inhibited glutamate release (n−3−5).

FIGS. 12A-B show astrocytic Ca²⁺ increases are associated with a joint release of organic osmolytes. FIG. 12A shows a time course of glutamate level in extracellular perfusion buffer showed that ATP stimulation (100 μM) triggers a 2- to 5-fold increase in glutamate release, whereas a 10-fold elevation of extracellular glutamate is evoked by hypotonicity (214 mOsm). HPLC analysis of the amino acid profile revealed that ATP stimulation caused release of glutamate, aspartate, glutamine, and taurine but not of asparagines, isoleucin, leucine, phenelalanine, and tyrosine. The hypoosmotic challenge triggered amino acid release of a larger amplitude, but the profile was almost identical to that observed after ATP stimulation. Amino acid release is displayed as changes from baseline concentration (n=9). FIG. 12B shows that BAPTA and NPPB not only inhibited ATP-induced release of glutamate but also the release of aspartate, glutamine, and taurine.

FIGS. 13A-E show astrocytic Ca²⁺ increases are associated with activation of a glutamate-permeable channel. FIG. 13A, left panel, shows ATP induced an inward current in cultured astrocytes. Astrocytes were patched in the whole-cell voltage-claim configuration with a holding potential of −60 mV. Continuous recording with 123 mM Cs-glutamate in the pipette and 250 mM sucrose in the extracellular solution showed that ATP evoked an inward current, indicated as (1). When Cs-gluconate replaced intracellular Cs-glutamate and sucrose in extracellular solution, no currents were induced by ATP (2). When aCSF was replaced with 126 mM NaCl in the bath, the amplitude of the inward current increased (3). When 126 mM NMDG-Cl replaced sucrose, a similar inward current was induced by ATP (4). When K-gluconate replaced intracellular Cs-glutamate, a small outward current was recorded (5). The addition of 10 mM BAPTA in the pipette (same conditions as in 3) inhibited the inward current (6). When 100 μM NPPB was added to the bath (same conditions as in 3), the inward current was inhibited (7). FIG. 13A, right panel, shows mean amplitude of ATP-induced currents. Replacing Cl⁻ with F (NaI) potently inhibited the inward current. Increasing the bath osmolarity by 15% (+15% Osm) by adding sucrose to the bath solution attenuated the ATP-induced current, whereas OxATP (300 μM for 1 h) was without effect. The ATP-induced current was inhibited by gossypol (10 μM) and FFA (100 μM). *, P<0.05 and **, P<0.01 compared with α. The numbers indicate responding cells/total cells in each experiment. FIG. 13B, upper panel, shows that the ramp I-V currents (ATP-induced net currents) with [CsGlu]ᵢₙ/[sucrose]ₒᵤₜ(a), [CsGlu]ᵢₙ/[NMDG]ₒᵤₜ (b), [KGlu]ᵢₙ/[NMDG]ₒᵤₜ(c), [CsGlu]ᵢₙ/[NaGluconate]ₒᵤₜ (d), [CsGlu]ᵢₙ/[CholineCl]ₒᵤₜ (e). FIG. 13B, lower panel shows a summary table of the reversal potentials (mean±SEM in mV). FIG. 13C shows measurements of reversal potential by using steady-state holding potentials in the same conditions as in FIG. 13B (a-e labeling as in FIG. 13B). The numbers to the left side of each trace show the holding potential, whereas the numbers on the right show the number of responding cells/the total number of tested cells. FIG. 13D shows ATP-induced Ca²⁺ increases in astrocytes in hippocampal slices (P14). Ca²⁺ normalized within 1 or 2 min, but some cells continued to display oscillatory increases in Ca²⁺ (white arrows). (Scale bar: 10 μm.) FIG. 13E shows ATP-evoked inward currents in astrocytes in hippocampal slices. FIG. 13E, left panel, show astrocytes in hippocampus (stratum radiatum) were identified under DIC optics by their small cell bodies (white arrowhead), which stained positive for GFAP (red), and by their high resting membrane potential, and absence of depolarization-evoked action potential. FIG. 13E, middle panel, shows representative recordings of ATP-induced currents. (a) indicates 50 mM K-glutamate/73 mM K-gluconate in the pipette and 126 mM NaCl in the bath. (b) indicates 10 mM BAPTA in the pipette with the same solutions as in (a). (c) indicates NPPB (100 μM) was added to the bath. Tetrodotoxin (1 μM) was present in the bath. FIG. 13E, right panel, is a summary histogram showing the mean amplitude of the ATP-induced currents with the number of responding cells/the total number of tested cells. *, P<0.05 compared to a Mean+SEM.

### DETAILED DESCRIPTION OF THE INVENTION

The present invention relates to a method of treating or preventing epileptic seizures in a subject. The method involves administering an agent which interferes with glutamate, aspartate, and/or ATP release from astrocytes to the subject under conditions effective to treat or prevent epileptic seizures.

Astrocytes are primarily viewed as passive support cells, which perform important but perfunctory housekeeping tasks to optimize the environment for neural transmission. New evidence has questioned this concept by demonstrating that astrocytes can actively modulate neuronal function. Indeed, astrocytes are required for synapse formation and, stability and can actively modulate synaptic transmission by release of glutamate by exocytosis (Volterra et al., “Astrocytes, From Brain Glue to Communication Elements: The Revolution Continues,” Nat. Rev. Neurosci. 6(8):626-640 (2005); Haydon, P. G., “GLIA: Listening and Talking to the Synapse,” Nat. Rev. Neurosci. 2(3):185-193 (2001), which are hereby incorporated by reference in their entirety). Astrocytes express several proteins that are required for exocytosis, and neurotoxins inhibit astrocytic glutamate release in cultures. Astrocytes also express functional vesicular glutamate transporters VGLUT1/2 and pharmacological inhibition of VGLUT1/2 reduced Ca²⁺-dependent glutamate release (Montana et al., “Vesicular Glutamate Transporter-Dependent Glutamate Release From Astrocytes,” J. Neurosci. 24(12):2633-2642 (2004); Bezzi et al., “Astrocytes Contain a Vesicular Compartment That is Competent for Regulated Exocytosis of Glutamate,” Nat. Neurosci. 7(6):613-620 (2004), which are hereby incorporated by reference in their entirety). However, other mechanisms by which astrocytes release glutamate likely exist. In addition, astrocytes possess multiple mechanisms for several key functions. For example, the important task of K⁺ buffering is undertaken by several K⁺ channels expressed by astrocytes, including KIR4.1 and rSlo K(Ca) (Price et al., “Distribution of rSlo Ca²⁺-Activated K⁺ Channels in Rat Astrocyte Perivascular Endfeet,” Brain Res. 956(2):183-193 (2002), which is hereby incorporated by reference in its entirety), but also by the K⁺—Na⁺—Cl⁻ cotransporter (Su et al., “Contribution of Na(+)-K(+)-Cl(−) Cotransporter to High-[K(+)](o)-Induced Swelling and EAA Release in Astrocytes,” Am. J. Physiol. 282(5):C1136-C1146 (2002), which is hereby incorporated by reference in its entirety). The present invention utilizes these properties of astrocytes to treat and/or prevent epileptic seizures.

Glutamate is a small anion that permeates through several channels, including volume-sensitive channels (VSCs) (Mongin et al., “ATP Regulates Anion Channel-Mediated Organic Osmolyte Release From Cultured Rat Astrocytes via Multiple Ca²⁺-Sensitive Mechanisms,” Am. J. Physiol. 288(1):C204-C213 (2005), which is hereby incorporated by reference in its entirety). Furthermore, glutamate functions as an osmolyte and is released in large quantities by astrocytes in response to external hypotonicity (Kimelberg et al., “Swelling-Induced Release of Glutamate, Aspartate, and Taurine from Astrocyte Cultures,” J. Neurosci. 10(5):1583-1591 (1990), which is hereby incorporated by reference in its entirety). Cellular swelling leads to activation of VSCs and to the release of glutamate and other amino acids including aspartate, glutamine, and taurine, as a part of the regulatory volume decrease (Jentsch et al., “Molecular Structure and Physiological Function of Chloride Channels,” Physiol. Rev. 82(2):503-568 (2002), which is hereby incorporated by reference in its entirety). Ca²⁺-dependent astrocytic glutamate release has not been linked previously to the opening of VSCs, because these channels are activated by Ca²⁺-independent processes. The present invention shows that receptor-induced Ca²⁺ increase is associated with an increase in astrocytic cell volume, which leads to the activation of VSCs and, thereby, results in the Ca²⁺-dependent release of glutamate.

The methods of the present invention, when used to treat epilepsy, are particularly useful in reducing the incidence of and/or the spread of epileptic seizures. The agents which are administered can include those that do not suppress neural transmission.

In preferred embodiments, the agent interferes with glutamate release, aspartate release, and/or ATP release from astrocytes and includes compounds selected from those presented in Tables 1, 2, 3, 4, 5, or 6; all cited references are hereby incorporated by reference.

TABLE 1

| | Calcium Buffers | References |
|---|---|---|
| | Calcium-Binding Proteins: | |
| 1 | Calretinin (CR); | Schwaller B et al., Cerebellum. 2002 Dec; 1(4): 241-58 |
| 2 | Calbindin D-28k (CB); | Schwaller B et al., Cerebellum. 2002 Dec; 1(4): 241-58 |
| 3 | Parvalbumin (PV); | Schwaller B et al., Cerebellum. 2002 Dec; 1(4): 241-58 |
| 4 | Calcyclin (S100A6); | Lesniak W et al., Acta Neurobiol Exp (Wars). 2000; 60(4): 569-75 |
| 5 | hGCAP1 | Haeseleer F et al., Biochem Biophys Res Commun. 2002 Jan 18; |
| | | 290(2): 615-23 |
| 6 | hGCAP2 | Haeseleer F et al., Biochem Biophys Res Commun. 2002 Jan 18; |
| | | 290(2): 615-23 |
| 7 | hGCAP3 | Haeseleer F et al., Biochem Biophys Res Commun. 2002 Jan 18; |
| | | 290(2): 615-23 |
| 8 | hCaBP1 | Haeseleer F et al., Biochem Biophys Res Commun. 2002 Jan 18; |
| | | 290(2): 615-23 |
| 9 | mCaBP1 | Haeseleer F et al., Biochem Biophys Res Commun. 2002 Jan 18; |
| | | 290(2): 615-23 |
| 10 | hCaBP2 | Haeseleer F et al., Biochem Biophys Res Commun. 2002 Jan 18; |
| | | 290(2): 615-23 |
| 11 | mCaBP2 | Haeseleer F et al., Biochem Biophys Res Commun. 2002 Jan 18; |
| | | 290(2): 615-23 |
| 12 | hCaBP5 | Haeseleer F et al., Biochem Biophys Res Commun. 2002 Jan 18; |
| | | 290(2): 615-23 |
| 13 | mCaBP5 | Haeseleer F et al., Biochem Biophys Res Commun. 2002 Jan 18; |
| | | 290(2): 615-23 |
| 14 | Recoverin | Haeseleer F et al., Biochem Biophys Res Commun. 2002 Jan 18; |
| | | 290(2): 615-23 |
| 15 | Visinin | Haeseleer F et al., Biochem Biophys Res Commun. 2002 Jan 18; |
| | | 290(2): 615-23 |
| 16 | VILIP1: human visinin-like protein 1 | Haeseleer F et al., Biochem Biophys Res Commun. 2002 Jan 18; |
| | | 290(2): 615-23 |
| 17 | VILIP2: rat visinin-like protein 2 | Haeseleer F et al., Biochem Biophys Res Commun. 2002 Jan 18; |
| | | 290(2): 615-23 |
| 18 | VILIP3: human visinin-like protein 3 | Haeseleer F et al., Biochem Biophys Res Commun. 2002 Jan 18; |
| | | 290(2): 615-23 |
| 19 | NCS-1: rat neuronal calcium sensor 1 | Haeseleer F et al., Biochem Biophys Res Commun. 2002 Jan 18; |
| | | 290(2): 615-23 |
| 20 | Neurocalcin | Haeseleer F et al., Biochem Biophys Res Commun. 2002 Jan 18; |
| | | 290(2): 615-23 |
| 21 | Hippocalcin | Haeseleer F et al., Biochem Biophys Res Commun. 2002 Jan 18; |
| | | 290(2): 615-23 |
| 22 | CaM-like protein | Haeseleer F et al., Biochem Biophys Res Commun. 2002 Jan 18; |
| | | 290(2): 615-23 |
| 23 | CaM | Haeseleer F et al., Biochem Biophys Res Commun. 2002 Jan 18; |
| | | 290(2): 615-23 |
| 24 | GCIP | Haeseleer F et al., Biochem Biophys Res Commun. 2002 Jan 18; |
| | | 290(2): 615-23 |
| 25 | KChIP1 | Haeseleer F et al., Biochem Biophys Res Commun. 2002 Jan 18; |
| | | 290(2): 615-23 |
| 26 | KChIP2 | Haeseleer F et al., Biochem Biophys Res Commun. 2002 Jan 18; |
| | | 290(2): 615-23 |
| 27 | KChIP3 | Haeseleer F et al., Biochem Biophys Res Commun. 2002 Jan 18; |
| | | 290(2): 615-23 |
| | GCAP: guanylate cyclase-activating | |
| | protein | |
| | CaBP: Calcium-binding proteins | |
| | CaM: Calmodulin | |
| | Prefix h: Homo sapiens | |
| | Prefix m: Mus musculus | |
| 28 | Calcineurin | Ikura M et al., BioEssays 24: 625-636, 2002 |
| | Calcium Chelators: | |
| 29 | 1,2-bis (2-aminophenoxy) Ethane- | Ouanounou A et al., Journal of Neuroscience, Feb. 1, 1999, |
| | N,N,N′,N′-tetraacetic acid (BAPTA) | 19(3): 906-915 |
| 30 | Ethylene glycol-bis(β-aminoethyl)- | |
| | N,N,N′N′-tetraacetic acid (EGTA) | |
| 31 | Ethylenediamine tetra-acetate (EDTA) | |
| | Calcium Channel Blockers: | |
| 32 | Nifedipine | Kochegarov AA, Cell Calcium. 2003 Mar; 33(3): 145-62 |
| 33 | Verapamil | Kochegarov AA, Cell Calcium. 2003 Mar; 33(3): 145-62 |
| 34 | Diltiazem | Kochegarov AA, Cell Calcium. 2003 Mar; 33(3): 145-62 |
| 35 | BAY K 8644 | Kochegarov AA, Cell Calcium. 2003 Mar; 33(3): 145-62 |
| 36 | SDZ-202 791 | Kochegarov AA, Cell Calcium. 2003 Mar; 33(3): 145-62 |
| 37 | Nicardipine | Kochegarov AA, Cell Calcium. 2003 Mar; 33(3): 145-62 |
| 38 | Nimodipine | Kochegarov AA, Cell Calcium. 2003 Mar; 33(3): 145-62 |
| 39 | Isradipine | Kochegarov AA, Cell Calcium. 2003 Mar; 33(3): 145-62 |
| 40 | Amlodipine besylate | Kochegarov AA, Cell Calcium. 2003 Mar; 33(3): 145-62 |
| 41 | Vatanidipine | Kochegarov AA, Cell Calcium. 2003 Mar; 33(3): 145-62 |
| 42 | Iganidipine | Kochegarov AA, Cell Calcium. 2003 Mar; 33(3): 145-62 |
| 43 | Lacidipine | Kochegarov AA, Cell Calcium. 2003 Mar; 33(3): 145-62 |
| 44 | Nilvadipine | Kochegarov AA, Cell Calcium. 2003 Mar; 33(3): 145-62 |
| 45 | Lemidipine | Kochegarov AA, Cell Calcium. 2003 Mar; 33(3): 145-62 |
| 46 | Aranidipine | Kochegarov AA, Cell Calcium. 2003 Mar; 33(3): 145-62 |
| 47 | Sipatrigine (BW619C89) | Kochegarov AA, Cell Calcium. 2003 Mar; 33(3): 145-62 |
| 48 | NS-7 | Kochegarov AA, Cell Calcium. 2003 Mar; 33(3): 145-62 |
| 49 | (R-(—)-2,4-diamino-6-(fluromethyl)-5- | Kochegarov AA, Cell Calcium. 2003 Mar; 33(3): 145-62 |
| | (2,3,5-trichlorophenyl)pyrimidine) | |
| | (202W92) | |
| 50 | Lamotrigine | Kochegarov AA, Cell Calcium. 2003 Mar; 33(3): 145-62 |
| 51 | Flunarizine | Kochegarov AA, Cell Calcium. 2003 Mar; 33(3): 145-62 |
| 52 | Cinnarizine | Kochegarov AA, Cell Calcium. 2003 Mar; 33(3): 145-62 |
| 53 | Lidoflazine | Kochegarov AA, Cell Calcium. 2003 Mar; 33(3): 145-62 |
| 54 | Dotarizine | Kochegarov AA, Cell Calcium. 2003 Mar; 33(3): 145-62 |
| 55 | Aligeron | Kochegarov AA, Cell Calcium. 2003 Mar; 33(3): 145-62 |
| 56 | Fluspirilene | Kochegarov AA, Cell Calcium. 2003 Mar; 33(3): 145-62 |
| 57 | Pimozide | Kochegarov AA, Cell Calcium. 2003 Mar; 33(3): 145-62 |
| 58 | Penfluridol | Kochegarov AA, Cell Calcium. 2003 Mar; 33(3): 145-62 |
| 59 | Lomerizine | Kochegarov AA, Cell Calcium. 2003 Mar; 33(3): 145-62 |
| 60 | AH-1058 | Kochegarov AA, Cell Calcium. 2003 Mar; 33(3): 145-62 |
| 61 | Amiloride | Kochegarov AA, Cell Calcium. 2003 Mar; 33(3): 145-62 |
| 62 | ω-Conotoxin GVIA, Conus geographus | Kochegarov AA, Cell Calcium. 2003 Mar; 33(3): 145-62 |
| 63 | ω-Conotoxin MVIIA, (SNX-111), | Kochegarov AA, Cell Calcium. 2003 Mar; 33(3): 145-62 |
| | Conus magus | |
| 64 | ω-Conotoxin SVIB, Conus striatus | Kochegarov AA, Cell Calcium. 2003 Mar; 33(3): 145-62 |
| 65 | ω-Conotoxin MVIIC, (SNX-230), | Kochegarov AA, Cell Calcium. 2003 Mar; 33(3): 145-62 |
| | Conus magus | |
| 66 | ω-Agatoxin IVA, Agelenopsis aperta | Kochegarov AA, Cell Calcium. 2003 Mar; 33(3): 145-62 |
| 67 | ω-Agatoxin TK, Agelenopsis aperta | Kochegarov AA, Cell Calcium. 2003 Mar; 33(3): 145-62 |
| 68 | ω-Agatoxin IIIA | Kochegarov AA, Cell Calcium. 2003 Mar; 33(3): 145-62 |
| 69 | FTX, synthetic analog Agelenopsis | Kochegarov AA, Cell Calcium. 2003 Mar; 33(3): 145-62 |
| | aperta toxin | |
| 70 | Calcicludine, Dendroaspis angusticeps | Kochegarov AA, Cell Calcium. 2003 Mar; 33(3): 145-62 |
| 71 | Calciseptine, Dendroaspis polylepis | Kochegarov AA, Cell Calcium. 2003 Mar; 33(3): 145-62 |
| | polylepis | |
| 72 | FS-2, Dendroaspis polylepis polylepis | Kochegarov AA, Cell Calcium. 2003 Mar; 33(3): 145-62 |
| 73 | TaiCatoxin, Oxyuranus scutellatus | Kochegarov AA, Cell Calcium. 2003 Mar; 33(3): 145-62 |
| | scutellatus | |
| 74 | Bepridil | Kochegarov AA, Cell Calcium. 2003 Mar; 33(3): 145-62 |
| 75 | Mibefradil | Kochegarov AA, Cell Calcium. 2003 Mar; 33(3): 145-62 |
| 76 | Astemizole | Kochegarov AA, Cell Calcium. 2003 Mar; 33(3): 145-62 |
| 77 | Cisapride | Kochegarov AA, Cell Calcium. 2003 Mar; 33(3): 145-62 |
| 78 | Terfenadine | Kochegarov AA, Cell Calcium. 2003 Mar; 33(3): 145-62 |
| 79 | Gentamicin | Kochegarov AA, Cell Calcium. 2003 Mar; 33(3): 145-62 |
| 80 | Streptomycin | Kochegarov AA, Cell Calcium. 2003 Mar; 33(3): 145-62 |
| 81 | Netilmicin | Kochegarov AA, Cell Calcium. 2003 Mar; 33(3): 145-62 |
| 82 | Amikacin | Kochegarov AA, Cell Calcium. 2003 Mar; 33(3): 145-62 |
| 83 | Sisomicin | Kochegarov AA, Cell Calcium. 2003 Mar; 33(3): 145-62 |
| 84 | Dactimicin | Kochegarov AA, Cell Calcium. 2003 Mar; 33(3): 145-62 |
| 85 | Kanamycin | Kochegarov AA, Cell Calcium. 2003 Mar; 33(3): 145-62 |
| 86 | Kanendomycin | Kochegarov AA, Cell Calcium. 2003 Mar; 33(3): 145-62 |
| 87 | Tobramycin | Kochegarov AA, Cell Calcium. 2003 Mar; 33(3): 145-62 |
| 88 | Dibekacin | Kochegarov AA, Cell Calcium. 2003 Mar; 33(3): 145-62 |
| 89 | Tetrahydropalmatine (THP) | Kochegarov AA, Cell Calcium. 2003 Mar; 33(3): 145-62 |
| 90 | Tetrandrine | Kochegarov AA, Cell Calcium. 2003 Mar; 33(3): 145-62 |
| 91 | 1-[1-[(6-Methoxy)-naphth-2-yl]]-ethyl- | Kochegarov AA, Cell Calcium. 2003 Mar; 33(3): 145-62 |
| | 2-(1-piperidinyl)-acetyl-6,7-dimethoxy- | |
| | 1,2,3,4-tetrahydroisoquinoline (CPU- | |
| | 23) | |
| 92 | SKF 96365 | Kochegarov AA, Cell Calcium. 2003 Mar; 33(3): 145-62 |
| 93 | Pinaverium | Kochegarov AA, Cell Calcium. 2003 Mar; 33(3): 145-62 |
| 94 | Ethosuximide | Kochegarov AA, Cell Calcium. 2003 Mar; 33(3): 145-62 |
| 95 | Phenytoin | Kochegarov AA, Cell Calcium. 2003 Mar; 33(3): 145-62 |
| 96 | α-methyl-α-phenylsuccinimide | Kochegarov AA, Cell Calcium. 2003 Mar; 33(3): 145-62 |
| 97 | Valproic acid | Kochegarov AA, Cell Calcium. 2003 Mar; 33(3): 145-62 |
| 98 | Thiopental | Kochegarov AA, Cell Calcium. 2003 Mar; 33(3): 145-62 |
| 99 | Pentobarbital | Kochegarov AA, Cell Calcium. 2003 Mar; 33(3): 145-62 |
| 100 | Methohexital | Kochegarov AA, Cell Calcium. 2003 Mar; 33(3): 145-62 |
| 101 | Phenobarbital | Kochegarov AA, Cell Calcium. 2003 Mar; 33(3): 145-62 |
| 102 | Propofol | Kochegarov AA, Cell Calcium. 2003 Mar; 33(3): 145-62 |
| 103 | Octanol | Kochegarov AA, Cell Calcium. 2003 Mar; 33(3): 145-62 |
| 104 | Etomidate | Kochegarov AA, Cell Calcium. 2003 Mar; 33(3): 145-62 |
| 105 | Isoflurane | Kochegarov AA, Cell Calcium. 2003 Mar; 33(3): 145-62 |
| 106 | Halothane | Kochegarov AA, Cell Calcium. 2003 Mar; 33(3): 145-62 |
| 107 | Ketamine | Kochegarov AA, Cell Calcium. 2003 Mar; 33(3): 145-62 |
| 108 | 5-nitro-2-(3- | Kochegarov AA, Cell Calcium. 2003 Mar; 33(3): 145-62 |
| | phenylpropylamino)benzoic acid | |
| | (NPPB) | |
| 109 | Indanyloxyacetic acid 94 (IAA-94) | Kochegarov AA, Cell Calcium. 2003 Mar; 33(3): 145-62 |
| 110 | ω-conotoxin MVIIC | Kochegarov AA, Cell Calcium. 2003 Mar; 33(3): 145-62 |

TABLE 2
Potassium Channel Blockers

| | | |
|---|---|---|
| 1 | Tetraethylammonium (TEA) | Mathie A. et al., Gen Pharmacol. 1998 Jan; 30(1): 13-24 |
| 2 | 4-Aminopyridine (4-AP) | Mathie A. et al., Gen Pharmacol. 1998 Jan; 30(1): 13-24 |
| 3 | Cesium (Cs⁺) | Lesage A., Neuropharmacology 44 (2003) 1-7 |
| 4 | Barium (Ba²⁺) | Lesage A., Neuropharmacology 44 (2003) 1-7 |
| 5 | α-Dendrotoxin (α-DTx); | Mathie A. et al., Gen Pharmacol. 1998 Jan; 30(1): 13-24 |
| 6 | Charybdotoxin | Mathie A. et al., Gen Pharmacol. 1998 Jan; 30(1): 13-24 |
| 7 | Noxiustoxin | Mathie A. et al., Gen Pharmacol. 1998 Jan; 30(1): 13-24 |
| 8 | Nicardipine | Mathie A. et al., Gen Pharmacol. 1998 Jan; 30(1): 13-24 |
| 9 | Nifedipine | Mathie A. et al., Gen Pharmacol. 1998 Jan; 30(1): 13-24 |
| 10 | Nitrendipine | Mathie A. et al., Gen Pharmacol. 1998 Jan; 30(1): 13-24 |
| 11 | Nisoldipine (+) | Mathie A. et al., Gen Pharmacol. 1998 Jan; 30(1): 13-24 |
| 12 | (−) BAY K 8644 (−−) | Mathie A. et al., Gen Pharmacol. 1998 Jan; 30(1): 13-24 |
| 13 | Magnesium (Mg²⁺) | Mathie A. et al., Gen Pharmacol. 1998 Jan; 30(1): 13-24 |
| 14 | Calcium (Ca²⁺) | Mathie A. et al., Gen Pharmacol. 1998 Jan; 30(1): 13-24 |
| 15 | Cobalt (Co²⁺) | Mathie A. et al., Gen Pharmacol. 1998 Jan; 30(1): 13-24 |
| 16 | Manganese (Mn²⁺) | Mathie A. et al., Gen Pharmacol. 1998 Jan; 30(1): 13-24 |
| 17 | Nickel (Ni²⁺) | Mathie A. et al., Gen Pharmacol. 1998 Jan; 30(1): 13-24 |
| 18 | Cadmium (Cd²⁺) | Mathie A. et al., Gen Pharmacol. 1998 Jan; 30(1): 13-24 |
| 19 | Zinc (Zn²⁺) | Mathie A. et al., Gen Pharmacol. 1998 Jan; 30(1): 13-24 |
| 20 | Mercury (Hg²⁺) | Mathie A. et al., Gen Pharmacol. 1998 Jan; 30(1): 13-24 |
| 21 | Lanthanun (La³⁺) | Mathie A. et al., Gen Pharmacol. 1998 Jan; 30(1): 13-24 |
| 22 | Gadolinium ions (Gd³⁺) | Ferroni s et al., J Neurosci Res. 2003 May 1; 72(3): 363-72 |
| 23 | Caffeine | Mathie A. et al., Gen Pharmacol. 1998 Jan; 30(1): 13-24 |
| 24 | 3-isobutyl-1-methylxanthine (IBMX) | Mathie A. et al., Gen Pharmacol. 1998 Jan; 30(1): 13-24 |
| 25 | 1,2bis(2-aminophenoxy)ethane-N,N,N′,N′- | Mathie A. et al., Gen Pharmacol. 1998 Jan; 30(1): 13-24 |
| | tetraacetic acid (BAPTA) | |
| 26 | Quinine | Mathie A. et al., Gen Pharmacol. 1998 Jan; 30(1): 13-24 |
| 27 | Hydroquinidine | Mathie A. et al., Gen Pharmacol. 1998 Jan; 30(1): 13-24 |
| 28 | Quinacrine | Mathie A. et al., Gen Pharmacol. 1998 Jan; 30(1): 13-24 |
| 29 | Tacrine (Tacr) | Mathie A. et al., Gen Pharmacol. 1998 Jan; 30(1): 13-24 |
| 30 | Cyproheptadine (Cyp) | Mathie A. et al., Gen Pharmacol. 1998 Jan; 30(1): 13-24 |
| 31 | Amitriptyline (Amit) | Mathie A. et al., Gen Pharmacol. 1998 Jan; 30(1): 13-24 |
| 32 | Chlopromazine (CPZ) | Mathie A. et al., Gen Pharmacol. 1998 Jan; 30(1): 13-24 |
| 33 | Imipramine (Imip) | Mathie A. et al., Gen Pharmacol. 1998 Jan; 30(1): 13-24 |
| 34 | Phencyclidine (PCP) | Mathie A. et al., Gen Pharmacol. 1998 Jan; 30(1): 13-24 |
| 35 | 1,2,3,4,10-substituted acridin-9-ons | Bohuslavizki KH et al., Gen Physiol Biophys. 1993 Oct; 12(5): 491-6 |
| 36 | Psoralens | Bohuslavizki KH et al., Gen Physiol Biophys. 1994 Aug; 13(4): 309-28 |
| 37 | Benzofurans | Bohuslavizki KH et al., Gen Physiol Biophys. 1994 Aug; 13(4): 309-28 |
| 38 | Acridinons | Bohuslavizki KH et al., Gen Physiol Biophys. 1994 Aug; 13(4): 309-28 |
| 39 | Coumarins | Bohuslavizki KH et al., Gen Physiol Biophys. 1994 Aug; 13(4): 309-28 |
| 40 | Apamin | Roy ML, Sontheimer H, J Neurochem. 1995 Apr; 64(4): 1576-84 |
| 41 | Isoproterenol (ISO) | Roy ML, Sontheimer H, J Neurochem. 1995 Apr; 64(4): 1576-84 |
| 42 | Epinephrine (EPI) | Roy ML, Sontheimer H, J Neurochem. 1995 Apr; 64(4): 1576-84 |
| 43 | Forskolin | Roy ML, Sontheimer H, J Neurochem. 1995 Apr; 64(4): 1576-84 |
| 44 | 5-hydroxydecanoate (5-HD) | Horiguchi T et al., Stroke. 2003 Apr; 34(4): 1015-20. Epub 2003 Mar 20 |
| 45 | ZD7288 | Appel SB, et al., J Pharmacol Exp Ther. 2003 Aug; 306(2): 437-46. Epub |
| | | 2003 Apr 29 |
| 46 | Glipizide | Wan Q, Biol Signals Recept. 1999 Jul-Oct; 8(4-5): 309-15 |
| 47 | Glibenclamide | Calabresi P, et al., J Cereb Blood Flow Metab. 1997 Oct; 17(10): 1121-6 |
| 48 | Tolbutamide | Calabresi P, et al., J Neurosci. 1997 Jun 15; 17(12): 4509-16 |
| 49 | Gliquidone | Haj-Dahmane S, et al., Brain Res. 1993 Jun 18; 614(1-2): 270-8 |

TABLE 3

| | Phospholipase A Inhibitors | References |
|---|---|---|
| 1 | Aristolochic acid | Vesce S et al., Journal of Neurochemistry, Volume 90 |
| | | Issue 3 Page 683 - August 200 |
| 2 | Arachidonyl-trifluoromethyl ketone (ATK or AACOCF3) | Vesce S et al., Journal of Neurochemistry, Volume 90 |
| | | Issue 3 Page 683 - August 200 |
| 3 | Bromoenol lactone (BEL); | Yagi K et al., Neurochem Int. 2004 Jul; 45(1): 39-47 |
| 4 | Para-bromophenacyl bromide (para-BPB); | Tanaka E et al., J Neurophysiol. 2003 Nov; 90(5): 3213-23. |
| | | Epub 2003 Aug 13. |
| 5 | Quinacrine | Judge RK et al., Toxicol Appl Pharmacol. 2002 Jun |
| | | 15; 181(3): 184-91. |

TABLE 4

| | Phospholipase C Inhibitors | References |
|---|---|---|
| 1 | Neomycin | Nishizaki T. J Pharmacol Sci. 2004 Feb; 94(2): 100-2. |
| 2 | (1-[6-[[17α-3-methoxyestra-1,3,5(10)-trien-17- | Nishizaki T. J Pharmacol Sci. 2004 Feb; 94(2): 100-2. |
| | yl]amino]hexyl]-1H-pyrrole-2,5-dione (U73122); | |
| 3 | Tricyclodecan-9-yl xanthogenate (D609); | Kim SG et al., Cell Mol Neurobiol. 2003 June; 23(3): 401-18 |
| 4 | 3-Nitrocoumarin | Tisi R et al., Cell Biochem Funct. 2001 Dec; 19(4): 229-35 |

TABLE 5

| compounds | type | channels | ref | | |
|---|---|---|---|---|---|
| 1,9-dideoxyforskolin (DDF) | anion channel blockers | | Strange K et al., Am J | Kirk K, J Membr | Nilius B and |
| | | | Physiol. 1996 Mar; | Biol. 1997 Jul | Droogmans G, Acta |
| | | | 270(3 Pt 1): C711-30 | 1; 158(1): 1-16 | Physiol Scand. |
| | | | | | 2003 |
| | | | | | Feb; 177(2): 119-47 |
| 2,5-Dichlorodiphenylamine-2- | anion channel blockers | CIC-2 | Strange K et al., Am J | Kirk K, J Membr | |
| carboxylicacid (DCDPC) | | | Physiol. 1996 Mar; | Biol. 1997 Jul | |
| | | | 270(3 Pt 1): C711-30 | 1; 158(1): 1-16 | |
| 4,4′-diisothiocyanostilbene-2,2′- | anion channel blockers | | Strange K et al., Am J | Kirk K, J Membr | Nilius B and |
| disulfonic acid (DIDS) | | | Physiol. 1996 Mar; | Biol. 1997 Jul | Droogmans G, |
| | | | 270(3 Pt 1): C711-30 | 1; 158(1): 1-16 | Physiol Rev. 2001 |
| | | | | | Oct; 81(4): 1415-59 |
| 4,4′-dinitrostilbene-2,2′- | anion channel blockers | | | | |
| disulfonic acid (DNDS) | | | | | |
| 4-acetamido-4′- | anion channel blockers | | | | Nilius B and |
| isothiocyanatostilbene-2,2′- | | | | | Droogmans G, |
| disulfonic acid (SITS) | | | | | Physiol Rev. 2001 |
| | | | | | Oct; 81(4): 1415-59 |
| 5-nitro-2-(3- | anion channel blockers | | Strange K et al., Am J | Kirk K, J Membr | Nilius B and |
| phenylpropylamino)benzoic acid | | | Physiol. 1996 Mar; | Biol. 1997 Jul | Droogmans G, |
| (NPPB) | | | 270(3 Pt 1): C711-30 | 1; 158(1): 1-16 | Physiol Rev. 2001 |
| | | | | | Oct; 81(4): 1415-59 |
| 9-anthracene carboxylic acid | anion channel blockers | CIC-2 | Strange K et al., Am J | | Nilius B and |
| (9AC) | | | Physiol. 1996 Mar; | | Droogmans G, |
| | | | 270(3 Pt 1): C711-30 | | Physiol Rev. 2001 |
| | | | | | Oct; 81(4): 1415-59 |
| Cd²⁺ | anion channel blockers | CIC-2 | | | |
| Dipyridamole | anion channel blockers | | | Kirk K, J Membr | |
| | | | | Biol. 1997 Jul | |
| | | | | 1; 158(1): 1-16 | |
| flufenamic acid | anion channel blockers | | | Kirk K, J Membr | |
| | | | | Biol. 1997 Jul | |
| | | | | 1; 158(1): 1-16 | |
| Furosemide | anion channel blockers | | | Kirk K, J Membr | |
| | | | | Biol. 1997 Jul | |
| | | | | 1; 158(1): 1-16 | |
| Niflumic acid | anion channel blockers | | | Kirk K, J Membr | Nilius B and |
| | | | | Biol. 1997 Jul | Droogmans G, |
| | | | | 1; 158(1): 1-16 | Physiol Rev. 2001 |
| | | | | | Oct; 81(4): 1415-59 |
| Pyridoxal-5-phosphate, 4- | anion channel blockers | | Davis-Amara EM. J | Kirk K, J Membr | |
| pyridoxic acid3, pyridoxal HCl, | | | Exp Zool 1997 | Biol. 1997 Jul | |
| other pyridoxal derivatives | | | 279: 456-461 | 1; 158(1): 1-16 | |
| Zn²⁺ | anion channel blockers | CIC-2 | | Kirk K, J Membr | |
| | | | | Biol. 1997 Jul | |
| | | | | 1; 158(1): 1-16 | |
| 4-(2-Butyl-6,7-dichlor-2- | anion channel blockers, | | Decher N. Br J | Bourke. J | Nilius B and |
| cyclopentyl-indan-1-on-5-yl)oxybutyric | selective | | Pharmacol 2001 | Neurosurg 1981 | Droogmans G, |
| acid (DCPIB) | | | 134: 1467-1479 | 55: 364-370 | Physiol Rev. 2001 |
| | | | | | Oct; 81(4): 1415-59 |
| Mefloquine | Antimalarials | | | | Nilius B and |
| | | | | | Droogmans G, |
| | | | | | Physiol Rev. 2001 |
| | | | | | Oct; 81(4): 1415-59 |
| Chlorpromazine | Calmodulin antagonists | | | Kirk K, J Membr | |
| | | | | Biol. 1997 Jul | |
| | | | | 1; 158(1): 1-16 | |
| N-(6-aminohexyl)-5-chloro-1- | Calmodulin antagonists | | | Kirk K, J Membr | |
| naphthalene-sulfonamide(W7) | | | | Biol. 1997 Jul | |
| | | | | 1; 158(1): 1-16 | |
| Pimozide | Calmodulin antagonists | | | Kirk K, J Membr | |
| | | | | Biol. 1997 Jul | |
| | | | | 1; 158(1): 1-16 | |
| Trifluoperazine | Calmodulin antagonists | | | Kirk K, J Membr | |
| | | | | Biol. 1997 Jul | |
| | | | | 1; 158(1): 1-16 | |
| Ba²⁺ | cation channel blockers | | | Kirk K, J Membr | |
| | | | | Biol. 1997 Jul | |
| | | | | 1; 158(1): 1-16 | |
| lanthanum (La³⁺) | cation channel blockers | | Strange K et al., Am J | Kirk K, J Membr | |
| | | | Physiol. 1996 Mar; | Biol. 1997 Jul | |
| | | | 270(3 Pt 1): C711-30 | 1; 158(1): 1-16 | |
| Quinidine | cation channel blockers | | | Kirk K, J Membr | Nilius B and |
| | | | | Biol. 1997 Jul | Droogmans G, |
| | | | | 1; 158(1): 1-16 | Physiol Rev. 2001 |
| | | | | | Oct; 81(4): 1415-59 |
| Quinine | cation channel blockers | | | Kirk K, J Membr | Nilius B and |
| | | | | Biol. 1997 Jul | Droogmans G, |
| | | | | 1; 158(1): 1-16 | Physiol Rev. 2001 |
| | | | | | Oct; 81(4): 1415-59 |
| Clomiphen | estrogen inhibitors | | | | Nilius B and |
| | | | | | Droogmans G, |
| | | | | | Physiol Rev. 2001 |
| | | | | | Oct; 81(4): 1415-59 |
| Nafoxidine | estrogen inhibitors | | | | Nilius B and |
| | | | | | Droogmans G, |
| | | | | | Physiol Rev. 2001 |
| | | | | | Oct; 81(4): 1415-59 |
| Tamoxifen and derivatives | estrogen inhibitors/ | | Strange K et al., Am J | Kirk K, J Membr | Nilius B and |
| | Calmodulin antagonists | | Physiol. 1996 Mar; | Biol. 1997 Jul | Droogmans G, |
| | | | 270(3 Pt 1): C711-30 | 1; 158(1): 1-16 | Physiol Rev. 2001 |
| | | | | | Oct; 81(4): 1415-59 |
| nordihydroguaiaretic acid | inhibitors cyclooxygenase/ | | | | Nilius B and |
| (NDGA) | lipoxygenase | | | | Droogmans G, |
| | | | | | Physiol Rev. 2001 |
| | | | | | Oct; 81(4): 1415-59 |
| Cinnamyl-3,4-dihydroxy- | Lipoxygenase/ | | | Kirk K, J Membr | |
| □alpha-cyanocinnamate | Cytochrome P450 | | | Biol. 1997 Jul | |
| | inhibitors | | | 1; 158(1): 1-16 | |
| Eicosatetraenoicacid (ETYA) | Lipoxygenase/ | | | Kirk K, J Membr | |
| | Cytochrome P450 | | | Biol. 1997 Jul | |
| | inhibitors | | | 1; 158(1): 1-16 | |
| Gossypol | Lipoxygenase/ | | | Kirk K, J Membr | Nilius B and |
| | Cytochrome P450 | | | Biol. 1997 Jul | Droogmans G, |
| | inhibitors | | | 1; 158(1): 1-16 | Physiol Rev. 2001 |
| | | | | | Oct; 81(4): 1415-59 |
| Ketoconazole | Lipoxygenase/ | | | Kirk K, J Membr | |
| | Cytochrome P450 | | | Biol. 1997 Jul | |
| | inhibitors | | | 1; 158(1): 1-16 | |
| Nordihydroguaiareticacid | Lipoxygenase/ | | | Kirk K, J Membr | |
| (NDGA) | Cytochrome P450 | | | Biol. 1997 Jul | |
| | inhibitors | | | 1; 158(1): 1-16 | |
| Phloretin | L-type Ca²⁺ channels | | | Kirk K, J Membr | |
| | blocker | | | Biol. 1997 Jul | |
| | | | | 1; 158(1): 1-16 | |
| 2,4-Dinitrophenol (DNP) | Metabolic inhibitors | | | Kirk K, J Membr | |
| | | | | Biol. 1997 Jul | |
| | | | | 1; 158(1): 1-16 | |
| 2-Deoxy-D-glucose | Metabolic inhibitors | | | Kirk K, J Membr | |
| | | | | Biol. 1997 Jul | |
| | | | | 1; 158(1): 1 | |
| Azide | Metabolic inhibitors | | | Kirk K, J Membr | |
| | | | | Biol. 1997 Jul | |
| | | | | 1; 158(1): 1 | |
| Carbonyl cyanide p- | Metabolic inhibitors | | | Kirk K, J Membr | |
| trifluoromethoxyphenyl- | | | | Biol. 1997 Jul | |
| hydrazone (FCCP) | | | | 1; 158(1): 1 | |
| Rotenone | Metabolic inhibitors | | | Kirk K, J Membr | |
| | | | | Biol. 1997 Jul | |
| | | | | 1; 158(1): 1 | |
| Bumetanide | Na⁺-K⁺-2Cl⁻ cotransport | | Walker VE. Am J | | |
| | inhibitors | | Physiol 1999 | | |
| | | | 276: C1432-1438 | | |
| Verapamil | P-glycoprotein inhibitors | | | | |
| 4-pyridoxic acid3 | Pyridoxal Derivatives | | | | |
| N-Ethylmaleimide | Sulfhydryl reagent | | | Kirk K, J Membr | |
| | | | | Biol. 1997 Jul | |
| | | | | 1; 158(1): 1-16 | |
| Mibefradil | T-type Ca²⁺ channel | | | | Nilius B. 1997 Br J |
| | blocker | | | | Pharmacol |
| | | | | | 121: 547-555 |
| Arachidonicacid | unsaturated fatty acids | | | Kirk K, J Membr | Nilius B and |
| | | | | Biol. 1997 Jul | Droogmans G, |
| | | | | 1; 158(1): 1-16 | Physiol Rev. 2001 |
| | | | | | Oct; 81(4): 1415-59 |
| Linoleicacid | unsaturated fatty acids | | | Kirk K, J Membr | |
| | | | | Biol. 1997 Jul | |
| | | | | 1; 158(1): 1-16 | |
| Calixarenes | | | | | Nilius B and |
| | | | | | Droogmans G, |
| | | | | | Physiol Rev. 2001 |
| | | | | | Oct; 81(4): 1415-59 |
| Chromones | | | | | Nilius B and |
| | | | | | Droogmans G, |
| | | | | | Physiol Rev. 2001 |
| | | | | | Oct; 81(4): 1415-59 |
| extracellular milimolar | | | Strange K et al., Am J | | Nilius B and |
| nucleotides | | | Physiol. 1996 Mar; | | Droogmans G, |
| | | | 270(3 Pt 1): C711-30 | | Physiol Rev. 2001 |
| | | | | | Oct; 81(4): 1415-590 |
| delta9-tetrahydrocannabinol | Cannabinoids | | Dhein S. Cardiovasc | | |
| | | | Res. 2004 May | | |
| | | | 1; 62(2): 287-98 | | |
| Oubain | cardiac glycosides | | D Dhein S. | | |
| | | | Cardiovasc Res. 2004 | | |
| | | | May 1; 62(2): 287-98 | | |
| Strophanthidin | cardiac glycosides | | Dhein S. Cardiovasc | | |
| | | | Res. 2004 May | | |
| | | | 1; 62(2): 287-98 | | |
| 2,3 butandione monoxime | dephosphrylating agents | | Dhein S. Cardiovasc | | |
| | | | Res. 2004 May | | |
| | | | 1; 62(2): 287-98 | | |
| 11,12-epoxyeicosatrienoic acid | Eicosanoids | | Dhein S. Cardiovasc | | |
| | | | Res. 2004 May | | |
| | | | 1; 62(2): 287-98 | | |
| thromboxane A2 | Eicosanoids | | Dhein S. Cardiovasc | | |
| | | | Res. 2004 May | | |
| | | | 1; 62(2): 287-98 | | |
| arachidonic acid | fatty acids | | Dhein S. Cardiovasc | | |
| | | | Res. 2004 May | | |
| | | | 1; 62(2): 287-98 | | |
| decaenoic acid | fatty acids | | Dhein S. Cardiovasc | | |
| | | | Res. 2004 May | | |
| | | | 1; 62(2): 287-98 | | |
| myristoleic acid | fatty acids | | Dhein S. Cardiovasc | | |
| | | | Res. 2004 May | | |
| | | | 1; 62(2): 287-98 | | |
| oleic acid | fatty acids | | Dhein S. Cardiovasc | | |
| | | | Res. 2004 May | | |
| | | | 1; 62(2): 287-98 | | |
| flufenamic acid | fenamates | | Dhein S. Cardiovasc | Spray DC, et al., | |
| | | | Res. 2004 May | Curr Drug Targets. | |
| | | | 1; 62(2): 287-98 | 2002 | |
| | | | | Dec; 3(6): 455-64. | |
| meclofenamic acid | fenamates | | Dhein S. Cardiovasc | Spray DC, et al., | |
| | | | Res. 2004 May | Curr Drug Targets. | |
| | | | 1; 62(2): 287-98 | 2002 | |
| | | | | Dec; 3(6): 455-64. | |
| niflumic acid | fenamates | | Dhein S. Cardiovasc | Spray DC, et al., | |
| | | | Res. 2004 May | Curr Drug Targets. | |
| | | | 1; 62(2): 287-98 | 2002 | |
| | | | | Dec; 3(6): 455-64. | |
| 18-alpha-glycyrrhetinic acid | glycyrrhizic acid | | Dhein S. Cardiovasc | Spray DC, et al., | |
| | metabolites | | Res. 2004 May | Curr Drug Targets. | |
| | | | 1; 62(2): 287-98 | 2002 | |
| | | | | Dec; 3(6): 455-64. | |
| 18-beta-glycyrrhetinic acid | glycyrrhizic acid | | Dhein S. Cardiovasc | Spray DC, et al., | |
| | metabolites | | Res. 2004 May | Curr Drug Targets. | |
| | | | 1; 62(2): 287-98 | 2002 | |
| | | | | Dec; 3(6): 455-64. | |
| Carbenoxolone | glycyrrhizic acid | | Dhein S. Cardiovasc | Spray DC, et al., | |
| | metabolites | | Res. 2004 May | Curr Drug Targets. | |
| | | | 1; 62(2): 287-98 | 2002 | |
| | | | | Dec; 3(6): 455-64. | |
| 2-aminoethoxydiphenyl borate | IP3-receptor blocker | | Dhein S. Cardiovasc | | |
| | | | Res. 2004 May | | |
| | | | 1; 62(2): 287-98 | | |
| Heptanol | lipophillic agents | | Dhein S. Cardiovasc | Spray DC, et al., | |
| | | | Res. 2004 May | Curr Drug Targets. | |
| | | | 1; 62(2): 287-98 | 2002 | |
| | | | | Dec; 3(6): 455-64. | |
| Octanol | lipophillic agents | | Dhein S. Cardiovasc | Spray DC, et al., | |
| | | | Res. 2004 May | Curr Drug Targets. | |
| | | | 1; 62(2): 287-98 | 2002 | |
| | | | | Dec; 3(6): 455-64. | |
| 1-oleoyl-2-acetyl-sn-glycerol | Metabolites | | Dhein S. Cardiovasc | | |
| | | | Res. 2004 May | | |
| | | | 1; 62(2): 287-98 | | |
| decrease ATP | Metabolites | | Dhein S. Cardiovasc | | |
| | | | Res. 2004 May | | |
| | | | 1; 62(2): 287-98 | | |
| Diacylglycerol | Metabolites | | Dhein S. Cardiovasc | | |
| | | | Res. 2004 May | | |
| | | | 1; 62(2): 287-98 | | |
| Ethrane | Narcotics | | Dhein S. Cardiovasc | | |
| | | | Res. 2004 May | | |
| | | | 1; 62(2): 287-98 | | |
| Halothane | Narcotics | | Dhein S. Cardiovasc | | |
| | | | Res. 2004 May | | |
| | | | 1; 62(2): 287-98 | | |
| isoflurane | narcotics | | Dhein S. Cardiovasc | | |
| | | | Res. 2004 May | | |
| | | | 1; 62(2): 287-98 | | |
| 12-O-tetradecanoyphorbol-13- | phorbol esters | | Dhein S. Cardiovasc | | |
| acetate | | | Res. 2004 May | | |
| | | | 1; 62(2): 287-98 | | |
| Staurosporine | PKC inhibitors | | Dhein S. Cardiovasc | | |
| | | | Res. 2004 May | | |
| | | | 1; 62(2): 287-98 | | |
| benzylquininium | quinine delivatives | | Spray DC, et al., Curr | | |
| | | | Drug Targets. 2002 | | |
| | | | Dec; 3(6): 455 | | |
| mefloquinine | quinine delivatives | | Spray DC, et al., Curr | | |
| | | | Drug Targets. 2002 | | |
| | | | Dec; 3(6): 455 | | |
| quinine | quinine delivatives | | Spray DC, et al., Curr | | |
| | | | Drug Targets. 2002 | | |
| | | | Dec; 3(6): 455 | | |
| angiotensin-II | receptor ligands | | Dhein S. Cardiovasc | | |
| | | | Res. 2004 May | | |
| | | | 1; 62(2): 287-98 | | |
| atrial natriuretic factor | receptor ligands | | Dhein S. Cardiovasc | | |
| | | | Res. 2004 May | | |
| | | | 1; 62(2): 287-98 | | |
| Glibenclamide | | | | | |
| intracellular Mg2+ | | | | | Nilius B and |
| | | | | | Droogmans G, |
| | | | | | Physiol Rev. 2001 |
| | | | | | Oct; 81(4): 1415-59 |
| L644-711 | | maxi | Strange K et al., Am J | | |
| | | | Physiol. 1996 Mar; | | |
| | | | 270(3 Pt 1): C711-30 | | |
| oxalon dye diBA-5-C4 | | | | | Nilius B and |
| | | | | | Droogmans G, |
| | | | | | Physiol Rev. 2001 |
| | | | | | Oct; 81(4): 1415-59 |
| pertussis toxin | | maxi | Strange K et al., Am J | | |
| | | | Physiol. 1996 Mar; | | |
| | | | 270(3 Pt 1): C711 | | |
| Phalloidin | | maxi | Strange K et al., Am J | | |
| | | | Physiol. 1996 Mar; | | |
| | | | 270(3 Pt 1): C711 | | |
| PKC inhibitors | | maxi | Strange K et al., Am J | Kirk K, J Membr | |
| | | | Physiol. 1996 Mar; | Biol. 1997 Jul | |
| | | | 270(3 Pt 1): C711 | 1; 158(1): 1-16 | |
| carbacho | receptor ligands | | Dhein S. Cardiovasc | | |
| | | | Res. 2004 May | | |
| | | | 1; 62(2): 287-98 | | |
| FGF-2 | receptor ligands | | Dhein S. Cardiovasc | | |
| | | | Res. 2004 May | | |
| | | | 1; 62(2): 287-98 | | |
| noradrenaline | receptor ligands | | Dhein S. Cardiovasc | | |
| | | | Res. 2004 May | | |
| | | | 1; 62(2): 287-98 | | |
| VEGF | receptor ligands | | Dhein S. Cardiovasc | | |
| | | | Res. 2004 May | | |
| | | | 1; 62(2): 287-98 | | |
| acetic acid | weak acid | | Dhein S. Cardiovasc | | |
| | | | Res. 2004 May | | |
| | | | 1; 62(2): 287-98 | | |
| propionic acid | weak acid | | Dhein S. Cardiovasc | | |
| | | | Res. 2004 May | | |
| | | | 1; 62(2): 287-98 | | |

TABLE 6

| Compounds | type | ref | |
|---|---|---|---|
| GF-120918 | acridone carboxamide | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| XR-9576 | Anthranilamide | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| Amioderone | Antiarrhythmics | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| lidocaine | antiarrhythmics | Varma MV, et al., Pharmacol Res. 2003 | |
| | | Oct; 48(4): 347-59 | |
| Quinidine | Antiarrhythmics | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| aureobasidine A | antibiotics | Varma MV, et al., Pharmacol Res. 2003 | |
| | | Oct; 48(4): 347-59 | |
| cefoperazone | antibiotics | Varma MV, et al., Pharmacol Res. 2003 | |
| | | Oct; 48(4): 347-59 | |
| ceftriazone | antibiotics | Varma MV, et al., Pharmacol Res. 2003 | |
| | | Oct; 48(4): 347-59 | |
| erythromycin | antibiotics | Varma MV, et al., Pharmacol Res. 2003 | |
| | | Oct; 48(4): 347-59 | |
| itraconazole | antibiotics | Varma MV, et al., Pharmacol Res. 2003 | |
| | | Oct; 48(4): 347-59 | |
| ketoconozole | antibiotics | Varma MV, et al., Pharmacol Res. 2003 | |
| | | Oct; 48(4): 347-59 | |
| Tamoxifen | Antiestrogen | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| chloroquine | antimalarials | Varma MV, et al., Pharmacol Res. 2003 | |
| | | Oct; 48(4): 347-597 | |
| emetine | antimalarials | Varma MV, et al., Pharmacol Res. 2003 | |
| | | Oct; 48(4): 347-59 | |
| hydroxychloroquine | antimalarials | Varma MV, et al., Pharmacol Res. 2003 | |
| | | Oct; 48(4): 347-59 | |
| quinacrine | antimalarials | Varma MV, et al., Pharmacol Res. 2003 | |
| | | Oct; 48(4): 347-59 | |
| Quinine | Antimalarials | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| bepridil | Ca channel blockers | Varma MV, et al., Pharmacol Res. 2003 | |
| | | Oct; 48(4): 347-59 | |
| diltiazem | Ca channel blockers | Varma MV, et al., Pharmacol Res. 2003 | |
| | | Oct; 48(4): 347-59 | |
| felodipine | Ca channel blockers | Varma MV, et al., Pharmacol Res. 2003 | |
| | | Oct; 48(4): 347-59 | |
| Nifedipine | Ca channel blockers | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| nisoldipine | Ca channel blockers | Varma MV, et al., Pharmacol Res. 2003 | |
| | | Oct; 48(4): 347-59 | |
| nitrendipine | Ca channel blockers | Varma MV, et al., Pharmacol Res. 2003 | |
| | | Oct; 48(4): 347-59 | |
| tiapamil | Ca channel blockers | Varma MV, et al., Pharmacol Res. 2003 | |
| | | Oct; 48(4): 347-59 | |
| Verapamil | Ca channel blockers | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| chloropromazine | calmodulin antagonists | Varma MV, et al., Pharmacol Res. 2003 | |
| | | Oct; 48(4): 347-59 | |
| Trifluoperazine | calmodulin antagonists | Varma MV, et al., Pharmacol Res. 2003 | Thomas H and Coley |
| | antipsychotic | Oct; 48(4): 347-59 | HM, Cancer Control. |
| | | | 2003 Mar- |
| | | | Apr; 10(2): 159-65 |
| actinomycin D | cancer therapeutics | Varma MV, et al., Pharmacol Res. 2003 | Thomas H and Coley |
| | | Oct; 48(4): 347-59 | HM, Cancer Control. |
| | | | 2003 Mar- |
| | | | Apr; 10(2): 159-65 |
| colchicines | cancer therapeutics | Varma MV, et al., Pharmacol Res. 2003 | |
| | | Oct; 48(4): 347-59 | |
| Daunorubicin | cancer therapeutics | Varma MV, et al., Pharmacol Res. 2003 | Thomas H and Coley |
| | | Oct; 48(4): 347-59 | HM, Cancer Control. |
| | | | 2003 Mar- |
| | | | Apr; 10(2): 159-65 |
| Doxorubicin | cancer therapeutics | Varma MV, et al., Pharmacol Res. 2003 | Thomas H and Coley |
| | | Oct; 48(4): 347-59 | HM, Cancer Control. |
| | | | 2003 Mar- |
| | | | Apr; 10(2): 159-65 |
| Etoposide | cancer therapeutics | Varma MV, et al., Pharmacol Res. 2003 | Thomas H and Coley |
| | | Oct; 48(4): 347-59 | HM, Cancer Control. |
| | | | 2003 Mar- |
| | | | Apr; 10(2): 159-65 |
| mithramycin | cancer therapeutics | Varma MV, et al., Pharmacol Res. 2003 | |
| | | Oct; 48(4): 347-59 | |
| mitomycin C | cancer therapeutics | Varma MV, et al., Pharmacol Res. 2003 | |
| | | Oct; 48(4): 347-59 | |
| podophyllotoxin | cancer therapeutics | Varma MV, et al., Pharmacol Res. 2003 | |
| | | Oct; 48(4): 347-59 | |
| puromycin | cancer therapeutics | Varma MV, et al., Pharmacol Res. 2003 | |
| | | Oct; 48(4): 347-59 | |
| taxol | cancer therapeutics | Varma MV, et al., Pharmacol Res. 2003 | |
| | | Oct; 48(4): 347-59 | |
| Topotecan | cancer therapeutics | Varma MV, et al., Pharmacol Res. 2003 | Thomas H and Coley |
| | | Oct; 48(4): 347-59 | HM, Cancer Control. |
| | | | 2003 Mar- |
| | | | Apr; 10(2): 159-65 |
| trimterene | cancer therapeutics | Varma MV, et al., Pharmacol Res. 2003 | |
| | | Oct; 48(4): 347-59 | |
| Vinblastine | cancer therapeutics | Varma MV, et al., Pharmacol Res. 2003 | Thomas H and Coley |
| | | Oct; 48(4): 347-59 | HM, Cancer Control. |
| | | | 2003 Mar- |
| | | | Apr; 10(2): 159-65 |
| Vincristine | cancer therapeutics | Varma MV, et al., Pharmacol Res. 2003 | Thomas H and Coley |
| | | Oct; 48(4): 347-59 | HM, Cancer Control. |
| | | | 2003 Mar- |
| | | | Apr; 10(2): 159-65 |
| GG918 | carboxamide derivative | Borst P et al., J Natl Cancer Inst. 2000 Aug | Varma MV, et al., |
| | | 16; 92(16): 1295-302 | Pharmacol Res. 2003 |
| | | | Oct; 48(4): 347-59 |
| Bepridil | coronary vasodilator | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| Dipyridamole | coronary vasodilator | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| PSC833 | cyclosporin A analogue | Borst P et al., J Natl Cancer Inst. 2000 Aug | Varma MV, et al., |
| | | 16; 92(16): 1295-302 | Pharmacol Res. 2003 |
| | | | Oct; 48(4): 347-59 |
| valspodar (psc-833) | cyclosporine a Analog | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| LY-335979 | Dibenzosuberane | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| BIBW22BS | dipyridamole analog | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| BCECF AM | fluorescent dyes | Varma MV, et al., Pharmacol Res. 2003 | |
| | | Oct; 48(4): 347-59 | |
| Fluoro-2 | fluorescent dyes | Varma MV, et al., Pharmacol Res. 2003 | |
| | | Oct; 48(4): 347-59 | |
| Fura-2 | fluorescent dyes | Varma MV, et al., Pharmacol Res. 2003 | |
| | | Oct; 48(4): 347-59 | |
| hoechst 33342 | fluorescent dyes | Varma MV, et al., Pharmacol Res. 2003 | |
| | | Oct; 48(4): 347-59 | |
| rhodamine 123 | fluorescent dyes | Varma MV, et al., Pharmacol Res. 2003 | |
| | | Oct; 48(4): 347-59 | |
| aldosterone | hormones | Varma MV, et al., Pharmacol Res. 2003 | |
| | | Oct; 48(4): 347-59 | |
| clomiphene | hormones | Varma MV, et al., Pharmacol Res. 2003 | |
| | | Oct; 48(4): 347-59 | |
| cortisol | hormones | Varma MV, et al., Pharmacol Res. 2003 | |
| | | Oct; 48(4): 347-59 | |
| deoxycoticosterone | hormones | Varma MV, et al., Pharmacol Res. 2003 | |
| | | Oct; 48(4): 347-59 | |
| hydrocortisone | hormones | Varma MV, et al., Pharmacol Res. 2003 | |
| | | Oct; 48(4): 347-59 | |
| predinisone | hormones | Varma MV, et al., Pharmacol Res. 2003 | |
| | | Oct; 48(4): 347-59 | |
| testosterone | hormones | Varma MV, et al., Pharmacol Res. 2003 | |
| | | Oct; 48(4): 347-59 | |
| cyclosporin H | immunosuppressive | Varma MV, et al., Pharmacol Res. 2003 | |
| | | Oct; 48(4): 347-59 | |
| cyclosporine A | Immunosuppressive | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| sirolimus | immunosuppressive | Varma MV, et al., Pharmacol Res. 2003 | |
| | | Oct; 48(4): 347-59 | |
| tacrolimus | immunosuppressive | Varma MV, et al., Pharmacol Res. 2003 | |
| | | Oct; 48(4): 347-59 | |
| reserpine | indole alkaloids | Varma MV, et al., Pharmacol Res. 2003 | |
| | | Oct; 48(4): 347-59 | |
| yohimbine | indole alkaloids | Varma MV, et al., Pharmacol Res. 2003 | |
| | | Oct; 48(4): 347-59 | |
| MK571 | leukotriene D4 | Borst P et al., J Natl Cancer Inst. 2000 Aug | |
| | antagonist | 16; 92(16): 1295-302 | |
| bupivacaine | local anesthetics | Varma MV, et al., Pharmacol Res. 2003 | |
| | | Oct; 48(4): 347-59 | |
| Dexniguldipine | nifedipine analog | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| gramicidine D | peptides | Varma MV, et al., Pharmacol Res. 2003 | |
| | | Oct; 48(4): 347-59 | |
| n-acetyl-leucyl- | peptides | Varma MV, et al., Pharmacol Res. 2003 | |
| leucinal | | Oct; 48(4): 347-59 | |
| valinomycin | peptides | Varma MV, et al., Pharmacol Res. 2003 | |
| | | Oct; 48(4): 347-59 | |
| VX-710 | piperidine carboxylate | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| Progesterone | Progestative | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| Cinchonine | quinine analog | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| MS-209 | quinine analog | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| cremophor-EL | surfactants | Varma MV, et al., Pharmacol Res. 2003 | |
| | | Oct; 48(4): 347-59 | |
| triton X-100 | surfactants | Varma MV, et al., Pharmacol Res. 2003 | |
| | | Oct; 48(4): 347-59 | |
| tween 80 | surfactants | Varma MV, et al., Pharmacol Res. 2003 | |
| | | Oct; 48(4): 347-59 | |
| Toremifene | tamoxifen analog | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| S-9788 | triazinopiperidine | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| desipramine | tricyclic antidepressants | Varma MV, et al., Pharmacol Res. 2003 | |
| | | Oct; 48(4): 347-59 | |
| trazadone | tricyclic antidepressants | Varma MV, et al., Pharmacol Res. 2003 | |
| | | Oct; 48(4): 347-59 | |
| trans-flupentixol | trifluoperazine anlog | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| Dexverapamil | verapamil analog | Varma MV, et al., Pharmacol Res. 2003 | Robert J and Jarry CJ, |
| | | Oct; 48(4): 347-59 | Med Chem. 2003 Nov |
| | | | 6; 46(23): 4805-17 |
| benzbromarone | | Borst P et al., J Natl Cancer Inst. 2000 Aug | |
| | | 16; 92(16): 1295-302 | |
| bisantrene | | Thomas H and Coley HM, Cancer Control. 2003 Mar- | |
| | | Apr; 10(2): 159-65 | |
| cisplatin | | Thomas H and Coley HM, Cancer Control. 2003 Mar- | |
| | | Apr; 10(2): 159-65 | |
| docetaxel | | Thomas H and Coley HM, Cancer Control. 2003 Mar- | |
| | | Apr; 10(2): 159-65 | |
| epirubicin | | Thomas H and Coley HM, Cancer Control. 2003 Mar- | |
| | | Apr; 10(2): 159-65 | |
| ethidium bromide | | Varma MV, et al., Pharmacol Res. 2003 | |
| | | Oct; 48(4): 347-59 | |
| homoharringtonine | | Thomas H and Coley HM, Cancer Control. 2003 Mar- | |
| | | Apr; 10(2): 159-65 | |
| ivermectin | | Varma MV, et al., Pharmacol Res. 2003 | |
| | | Oct; 48(4): 347-59 | |
| leukotriene C4 | | Borst P et al., J Natl Cancer Inst. 2000 Aug | |
| | | 16; 92(16): 1295-302 | |
| liposomes | | Varma MV, et al., Pharmacol Res. 2003 | |
| | | Oct; 48(4): 347-59 | |
| methotrexate | | Thomas H and Coley HM, Cancer Control. 2003 Mar- | |
| | | Apr; 10(2): 159-65 | |
| mitoxantrone | | Thomas H and Coley HM, Cancer Control. 2003 Mar- | |
| | | Apr; 10(2): 159-65 | |
| OC144093 | | Varma MV, et al., Pharmacol Res. 2003 | |
| | | Oct; 48(4): 347-59 | |
| paclitaxel | | Thomas H and Coley HM, Cancer Control. 2003 Mar- | |
| | | Apr; 10(2): 159-65 | |
| probenecid | | Borst P et al., J Natl Cancer Inst. 2000 Aug | |
| | | 16; 92(16): 1295-302 | |
| quercein | | Varma MV, et al., Pharmacol Res. 2003 | |
| | | Oct; 48(4): 347-59 | |
| R101933 | | Varma MV, et al., Pharmacol Res. 2003 | |
| | | Oct; 48(4): 347-59 | |
| S-decylglutathione | | Borst P et al., J Natl Cancer Inst. 2000 Aug | |
| | | 16; 92(16): 1295-302 | |
| SDZ | | Varma MV, et al., Pharmacol Res. 2003 | |
| | | Oct; 48(4): 347-59 | |
| SN-38 | | Thomas H and Coley HM, Cancer Control. 2003 Mar- | |
| | | Apr; 10(2): 159-65 | |
| sulfinpyrazone | | Borst P et al., J Natl Cancer Inst. 2000 Aug | |
| | | 16; 92(16): 1295-302 | |
| teniposide | | Thomas H and Coley HM, Cancer Control. 2003 Mar- | |
| | | Apr; 10(2): 159-65 | |
| terfindine | | Varma MV, et al., Pharmacol Res. 2003 | |
| | | Oct; 48(4): 347-59 | |
| tumor necrosis factor | | Varma MV, et al., Pharmacol Res. 2003 | |
| | | Oct; 48(4): 347-59 | |
| vinorelbine | | Thomas H and Coley HM, Cancer Control. 2003 Mar- | |
| | | Apr; 10(2): 159-65 | |
| vitamine A | | Varma MV, et al., Pharmacol Res. 2003 | |
| | | Oct; 48(4): 347-59 | |
| mefloquinine | | | |

Agents of the present invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active agents of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active agents may be incorporated with excipients and used in the form of tablets, capsules, elixirs, suspensions, syrups, and the like. Such compositions and preparations should contain at least 0.1% of active agent. The percentage of the agent in these compositions may, of course, be varied and may conveniently be between about 2% to about 60% of the weight of the unit. The amount of active agent in such therapeutically useful compositions is such that a suitable dosage will be obtained. Preferred compositions according to the present invention are prepared so that an oral dosage unit contains between about 1 and 250 mg of active agent.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active agents may also be administered parenterally. Solutions or suspensions of these active agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents of the present invention may also be administered directly to the airways in the form of an aerosol. For use as aerosols, the agents of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The present invention also relates to a method of inhibiting hypersynchronous burst activity of a large group of neurons. The method involves administering an agent which interferes with glutamate, aspartate, and/or ATP release from astrocytes to the group of neurons under conditions effective to inhibit hypersynchronous burst activity. The method can be carried out either in vivo or in vitro. In carrying out the in vivo embodiment of the present invention, the above-described formulations and modes of administration can be utilized.

In preferred embodiments, the agent interferes with glutamate release, aspartate release, and/or ATP release from astrocytes and includes compounds selected from those presented in Tables 1, 2, 3, 4, 5, or 6, as presented above.

A further aspect of the present invention relates to a method of identifying agents suitable for treating or preventing epileptic seizures. The method involves contacting astrocytes with one or more candidate compounds, evaluating the astrocytes for glutamate, aspartate, and/or ATP release, and then identifying the candidate compounds which interfere with glutamate, aspartate, and/or ATP release as agents potentially suitable for treating or preventing epileptic seizures. Evaluation of astrocytes may also include detecting calcium release. Detection may be accomplished by monitoring changes in intracellular Ca²⁺ levels and Ca²⁺ release using the fluorescence of indicator dyes such as indo or fura, or using confocal Ca²⁺ imaging (Didier et al., “Ca²⁺ Blinks: Rapid Nanoscopic Store Calcium Signaling”, PNAS 102:3099-3104 (2005); Grimaldi et al., “Mobilization of Calcium from Intracellular Stores, Potentiation of Neurotransmitter-Induced Calcium Transients, and Capacitative Calcium Entry by 4-Aminopyridine,” J. Neurosci. 21:3135-3143 (2001), which are hereby incorporated by reference in their entirety).

Preferably, astrocytes are evaluated for glutamate release, aspartate release, and/or ATP release.

### EXAMPLES

The following examples are provided to illustrate embodiments of the present invention but are by no means intended to limit its scope.

### Example 1

### Materials and Methods

Slice preparation, 2-photon laser scanning imaging, and photolysis: Hippocampal slices were prepared from Sprague-Dawley (SD) rats (P14-18) as previously described (Kang et al., “Astrocyte-Mediated Potentiation of Inhibitory Synaptic Transmission,” Nat. Neurosci. 1:683-692 (1998); Zonta et al., “Neuron-to-Astrocyte Signaling is Central to the Dynamic Control of Brain Microcirculation,” Nat. Neurosci. 6:43-50 (2003); Liu et al., “Astrocyte-Mediated Activation of Neuronal Kainate Receptors. Proc. Natl. Acad. Sci. USA 101:3172-3177 (2004), which are hereby incorporated by reference in their entirety). The slices were mounted in a perfusion chamber and viewed by a custom built laser scanning microscope (BX61WI, FV300, Olympus) attached to Mai Tai laser (SpectraPhysics, Inc.). For Ca²⁺ measurements, slices were loaded with the Ca²⁺ indicator, fluo-4/AM (10 M, 1.5 h; Molecular Probes). For uncaging experiments, NP-EGTA/AM (200 μM; Molecular Probes) was co-incubated with fluo-4 AM. Photolysis was carried out by a 3 μm diameter UV pulse delivered as 10 trains (2 pulses with a duration of 10 ms and an interval of 50 ms; 100-500 μW) (DPSS lasers, Inc; 355 nm, 1.0 W).

Culture preparation and Ca²⁺ imaging: Cultured astrocytes were prepared from P1 rat pups as previously described (Arcuino et al., “Intercellular Calcium Signaling Mediated by Point-Source Burst Release of ATP,” Proc. Natl. Acad. Sci. USA 99:9840-9845 (2002), which is hereby incorporated by reference in its entirety). Confluent monolayer cultures were loaded with the Ca²⁺ indicator fluo-4 (5 μM for 1 h) and Ca²⁺ signaling monitored by confocal microscopy (BioRad, MRC1034) (Takano et al., “Glutamate Release Promotes Growth of Malignant Gliomas,” Nat. Med. 7:1010-1015 (2001), which is hereby incorporated by reference). Maximum increase in fluo-4 intensity following stimulation occurred within 20-30 s and was normalized relative to baseline fluorescence.

Electrophysiology: Whole-cell recordings from CA1 pyramidal neurons and stratum radiatum astrocytes in hippocampal slices were performed as previously described (Liu et al., “Astrocyte-Mediated Activation of Neuronal Kainate Receptors. Proc. Natl. Acad. Sci. USA 101:3172-3177 (2004), which is hereby incorporated by reference in its entirety). The perfusion artificial cerebrospinal fluid (ACSF) contained (in mM): 125 NaCl, 5 KCl, 1.25 NaH₂PO₄, 2 MgCl₂, 2 CaCl₂, 10 glucose and 25 NaHCO₃, pH 7.4 when aerated with 95% O₂, 5% CO₂ (Valiante et al., “Coupling Potentials in CA1 Neurons During Calcium-Free-Induced Field Burst Activity,” J. Neurosci. 15:6946-6956 (1995), which is hereby incorporated by reference in its entirety). Membrane potentials were filtered at 1 kHz, digitized at 5 kHz by using an Axopatch 200B amplifier, a pCLAMP 8.2 program and DigiData 1332A interface (Axon Instruments, Foster City, Calif.). Field potential recordings were made in stratum radiatum and stratum pyramidale of CA1 in hippocampal slices as previously described (Valiante et al., “Coupling Potentials in CA1 Neurons During Calcium-Free-Induced Field Burst Activity,” J. Neurosci. 15:6946-6956 (1995), which is hereby incorporated by reference in its entirety). Recording signals were filtered at 1 kHz, digitized at 5 kHz. All experiments were performed at 32-34° C.

Microdialysis, EEG recordings, and HPLC Analysis of Amino Acid Release: Adult SD rats (220-250 g) were anesthetized by ketamine (60 mg/kg) and xylazine (10 mg/kg). Microdialysis probes with a built-in electrode for EEG recordings (Applied Neuroscience, London, UK) were stereotaxically implanted into the right dorsal hippocampus (from bregma: 3.0 mm rostral; 2.0 mm lateral; from dura: 3.5 mm vertical) and fixed to the skull using dental cement and perfused using a microinjection pump (Harvard Apparatus Inc. USA), at a rate of 2 μl/min (Mena et al., “In vivo Glutamine Hydrolysis in the Formation of Extracellular Glutamate in the Injured Rat Brain,” J. Neurosci. Res. 60:632-641 (2000), which is hereby incorporated by reference in its entirety). Seizure activity was induced by delivering 4-AP (5 mM) through the microdialysis probe. The amino acid content was analyzed after reaction with ophthaldialdehyde utilizing fluorometric detection (Mena et al., “In vivo Glutamine Hydrolysis in the Formation of Extracellular Glutamate in the Injured Rat Brain,” J. Neurosci. Res. 60:632-641 (2000), which is hereby incorporated by reference in its entirety). EEG (1-100 Hz) was recorded continuously by an amplifier (DP-311, Warner Instruments, Inc) (Ayala et al., “Expression of Heat Shock Protein 70 Induced by 4-Aminopyridine Through Glutamate-Mediated Excitotoxic Stress in Rat Hippocampus In vivo,” Neuropharmacology 45:649-660 (2003); Urenjak et al., “Kynurenine 3-Hydroxylase Inhibition in Rats: Effects on Extracellular Kynurenic Acid Concentration and N-Methyl-D-Aspartate-Induced Depolarisation in the Striatum,” J. Neurochem. 75:2427-2433 (2000), which are hereby incorporated by reference in their entirety), a pCLAMP 9.2 program and DigiData 1332A interface with an interval of 200 μs.

In vivo two-photon Imaging: Adult mice (25-30 g) were anesthetized with ketamine (60 mg/kg) and xylazine (10 mg/kg) injection and a femoral artery catheterized. A custom made metal frame was glued to the skull with dental acrylic cement. A craniotomy (3 mm in diameter), centered 1-2 mm posterior to bregma and 2-3 mm from midline was performed. Dura was removed and the exposed cortex loaded with fluo-4/am (2 mM, 1 hr) and in selected experiments, sulforhodamine 101 (100 μM, 10 min) (Nimmerjahn et al., “Sulforhodamine 101 as a Specific Marker of Astroglia in the Neocortex In vivo,” Nature Methods 1:1-7 (2004), which is hereby incorporated by reference in its entirety). Agarose (0.75%) in saline was poured into the craniotomy and a coverslip mounted. Valproate was administred i.p. 450 mg/kg, 30 min before imaging; gabapentin 200 mg/kg, 60 min before imaging; and Na⁺ phenyloin, 100 mg/kg, 90 min before imaging (Boothe, D. M., “Anticonvulsant Therapy in Small Animals,” Vet. Clin. North. Am. Small Anim. Pract. 28:411-448 (1998), which is hereby incorporated by reference in its entirety). A custom built microscope attached to Tsunami/Millinium laser (SpectraPhysics, Inc.) and a scanning box (FV300, Olympus) was utilized for two-photon imaging experiments. Electrodes filled with saline containing 100 mM 4-AP were inserted 100-150 μm from the pial surface for cortical EEG (CoEEG) recordings. CoEEG (1-100 Hz) was recorded continuously by an amplifier (700A, Axon Instruments Inc.) (Ayala et al., “Expression of Heat Shock Protein 70 Induced by 4-Aminopyridine Through Glutamate-Mediated Excitotoxic Stress in Rat Hippocampus In vivo,” Neuropharmacology 45:649-660 (2003); Urenjak et al., “Kynurenine 3-Hydroxylase Inhibition in Rats: Effects on Extracellular Kynurenic Acid Concentration and N-Methyl-D-Aspartate-Induced Depolarisation in the Striatum,” J. Neurochem. 75:2427-2433 (2000), which are hereby incorporated by reference in their entirety), and a pCLAMP 9.2 program and DigiData 1332A interface with an interval of 200 μs. The seizure was induced by puffing 4-AP (5-10 pulses of 5-10 ms at 10 psi, Picospitzer). ATP (50 mM) was delivered iontophoretically (100 nA, 15 sec) with an electrode (100-150 μm from surface).

Animals were artificially ventilated with a ventilator (SAR-830, CWE) and blood gasses, pCO₂ (30-50 mm Hg), O₂ (100-150 mm Hg), and pH (7.25-7.45), monitored with a pH/blood gas analyzer (Rapidlab 248, Bayer, samples 40 μl). Body temperature was maintained at 37° C. by a homeothermic blanket system (Harvard Apparatus). All experiments were approved by the Institution Animal Care and Use Committee of University of Rochester.

### Example 2

### PDSs Can Be Triggered by an Action Potential-independent Mechanism

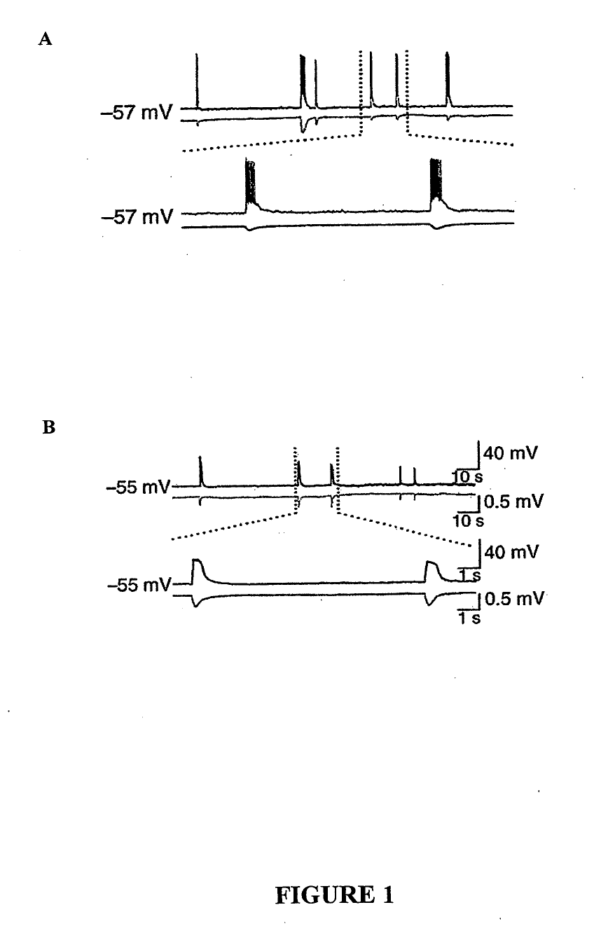

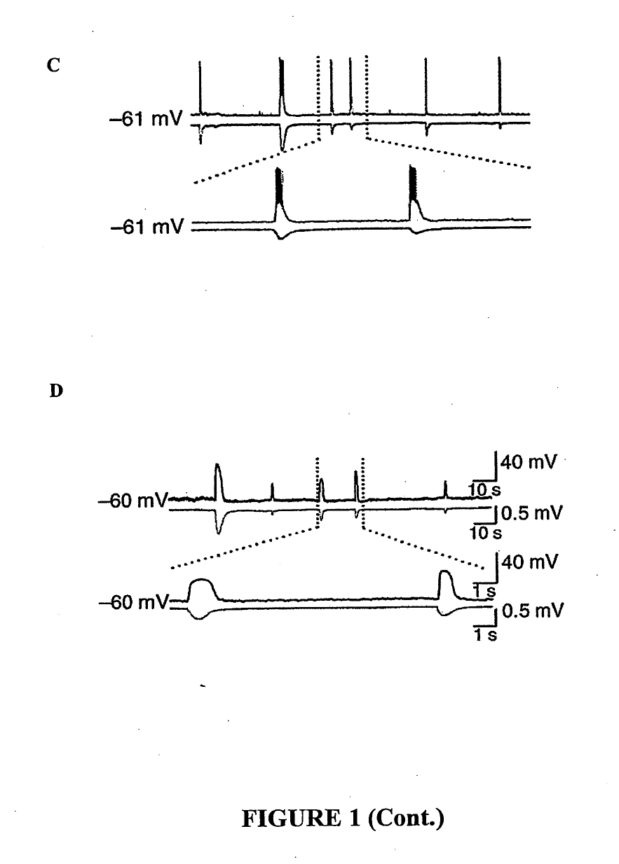

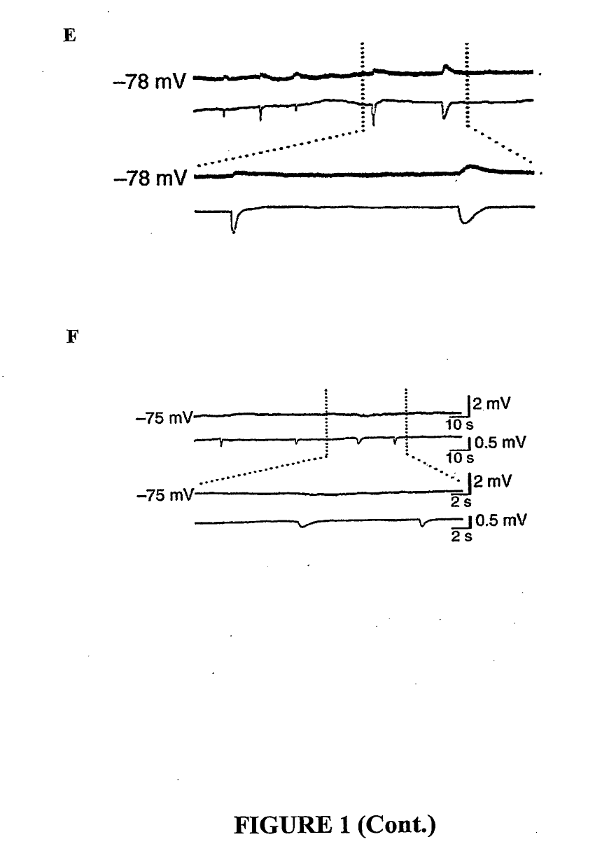

To examine the cellular mechanism underlying PDSs, CA1 pyramidal neurons in rat hippocampal slices exposed to 4-aminopyridine (4-AP) were patch clamped. 4-AP is a K⁺ channel blocker that induces intense electrical discharges in slices (Luhmann et al., “Generation and Propagation of 4-AP-Induced epileptiform activity in neonatal intact limbic structures in vitro. Eur. J. Neurosci. 12, 2757-2768 (2000), which is hereby incorporated by reference in its entirety) and seizure activity in experimental animals (Yamaguchi et al., “Effects of Anticonvulsant Drugs on 4-Aminopyridine-Induced Seizures in Mice,” Epilepsy Res. 11:9-16 (1992), which is hereby incorporated by reference in its entirety). All slices exposed to 4-AP (61 slices from 23 rats) exhibited epileptiform bursting activity expressed as transient episodes of neuronal depolarizations eliciting trains of action potentials (FIG. 1A). Bath application of TTX promptly eliminated neuronal firing (FIG. 1B). Unexpectedly, the paroxysmal neuronal depolarization events evoked by 4-AP were largely TTX-insensitive (FIG. 1B). Pyramidal neurons exposed to 4-AP continued to exhibit 10-30 mV depolarization shifts after addition of TTX, despite complete suppression of action potentials (FIG. 1B). To ensure that all synaptic activity was eliminated, a mixture of voltage-gated Ca²⁺ channel (VGCC) blockers, including nifedipine, mibefradil, omega-conotoxin MVIIC, omega-conotoxin GVIA, and SNX-482 was added (Elmslie, K. S., “Neurotransmitter Modulation of Neuronal Calcium Channels,” J. Bioenerg. Biomembr. 35:477-489 (2003), which is hereby incorporated by reference in its entirety). Notably, this cocktail of VGCC blockers did not suppress the expression of 4-AP-induced PDSs compared with TTX alone (FIG. 1B vs. FIG. 1D). In contrast to neurons, voltage changes in astrocytes during PDSs were minor, 0.5-2 mV, in accordance with the non-excitable properties of astrocytic plasma membranes, indicated in FIGS. 1E-F.

Combined, these experiments demonstrated that PDSs can be triggered by an action potential-independent mechanism. Neurons exhibited a 16±5 mV (n=24) depolarization shift, whereas astrocytes only display a modest change in membrane potential (0.5±0.2 mV, n=22) during PDSs in the presence of TTX.

### Example 3

### Glutamate Release Mediates Paroxysmal Depolarization Shifts

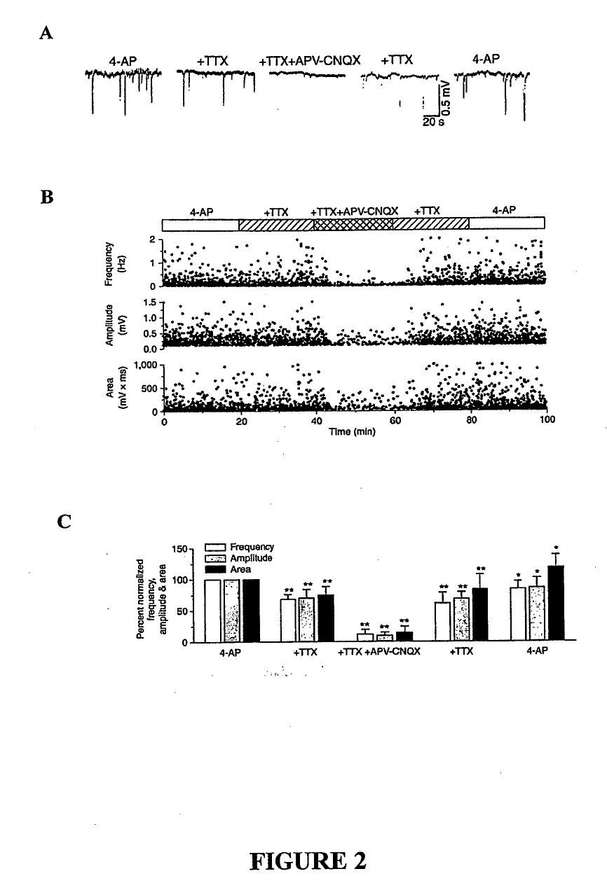

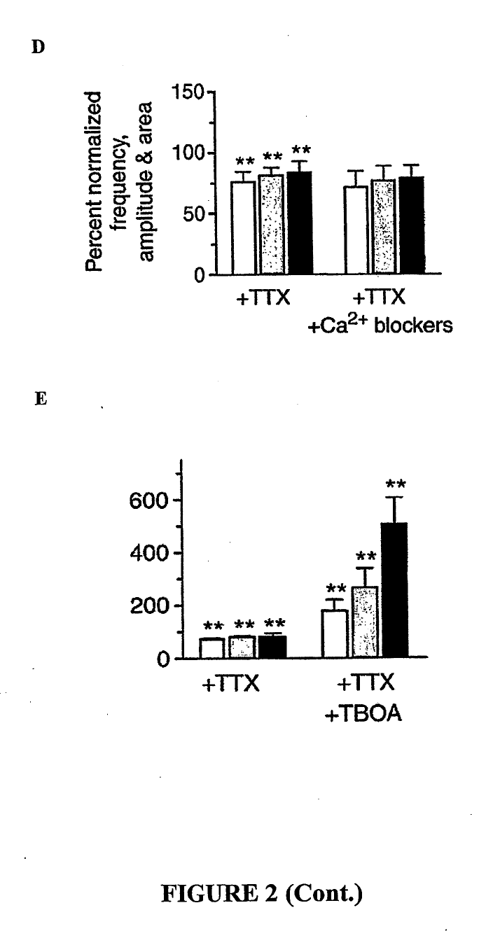

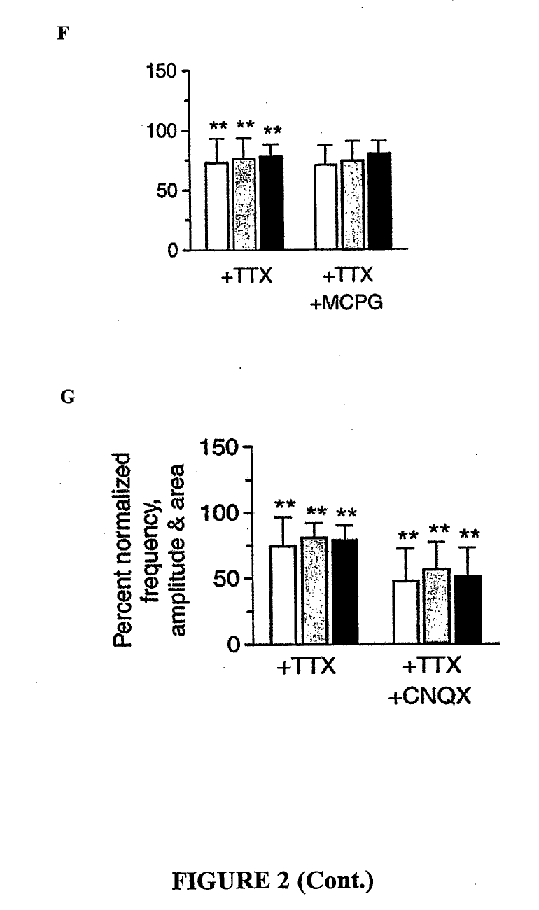

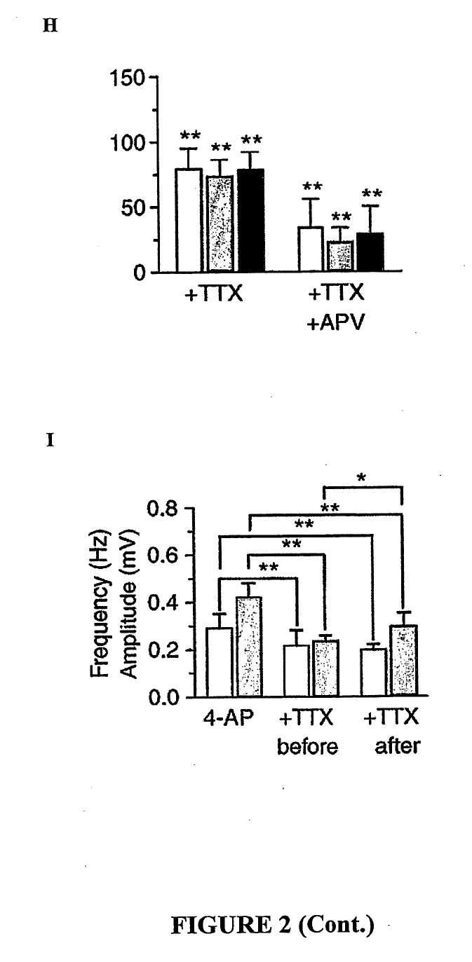

To examine the role of glutamate released from action potential-independent sources in PDSs, the occurrence of PDSs in the presence of TTX and GluR antagonists was quantified. The PDSs evoked by 4-AP resulted primarily from activation of ionotropic glutamate receptors, because APV and CNQX potently reduced both the frequency and the amplitude of the PDSs, in accordance with earlier studies (FIGS. 2A-C) (Meldrum, B. S., “Update on the Mechanism of Action of Antiepileptic Drugs,” Epilepsia 37 (Suppl.):6, S4-11 (1996), which is hereby incorporated by reference in its entirety). Washout of TTX, APV, and CNQX resulted in partial recovery of PDSs, as shown in FIGS. 2A-C. Addition of VGCC blockers did not cause an additional decrease in the frequency and amplitude of PDSs compared with TTX alone, further supporting the notion that glutamate was released from an action potential-independent source (FIG. 2D). PDSs persisted in the presence of D,L-threo-beta-benzyloxyaspartate (“TBOA”, a glutamate transport inhibitor), and TBOA increased the frequency and amplitude of PDSs significantly suggesting that inverted transport of glutamate did not contribute to PDSs (FIG. 2E).

Addition of TTX and CNQX (no APV) resulted in a significant decrease in the occurrence of PDSs compared with TTX alone (FIG. 2G), whereas TTX and APV compared with TTX alone, displayed a highly significant reduction in both frequency and amplitude of PDSs (FIG. 2H). Thus, the larger fraction (57%) of TTX-insensitive PDSs is caused by activation of NMDA receptors, whereas activation of AMPA receptors plays less of a significant role in generation of PDSs (26%). (S)-Alpha-methyl-4-carboxyphenylglycine ((S)-MCPG, a non-selective mGluR antagonist) (Drew et al., “Multiple Metabotropic Glutamate Receptor Subtypes Modulate GABAergic Neurotransmission in Rat Periaqueductal Grey Neurons In vitro,” Neuropharmacology 46:927-934 (2004), which is hereby incorporated by reference in its entirety) failed to reduce the frequency and amplitude of PDSs (FIG. 2F), indicating that the TTX-insensitive PDSs were not elicited by activation of mGluRs.

In all experiments thus far, TTX was first added after the hippocampal slices had been exposed for 20 min to 4-AP (FIGS. 1 and 2A-H). To test the possibility that astrocytic activation was secondary to neuronal bursting activity triggered by 4-AP, TTX (10-15 min) was added before exposing the slices to 4-AP (FIG. 2D. Interestingly, when TTX was added before 4-AP, the frequency and amplitude of 4-AP induced PDSs were only slightly decreased (FIG. 2I).

Combined, these observations show that TTX decreased the relative frequency of PDSs by 32±8% (P=0.001) compared with 4-AP alone (FIG. 2A-C). These observations suggest that 4-AP, in addition to its well known effects on neurons (Grimaldi et al., “Mobilization of Calcium from Intracellular Stores, Potentiation of Neurotransmitter-Induced Calcium Transients, and Capacitative Calcium Entry by 4-Aminopyridine,” J. Neurosci. 21:3135-3143 (2001), which is hereby incorporated by reference in its entirety), evoked a large number of paroxysmal depolarization events (approx 70% of total) which were triggered by release of glutamate from extrasynaptic sources.

### Example 4

### Paroxysmal Depolarization Shifts in Several Acute Seizure Models

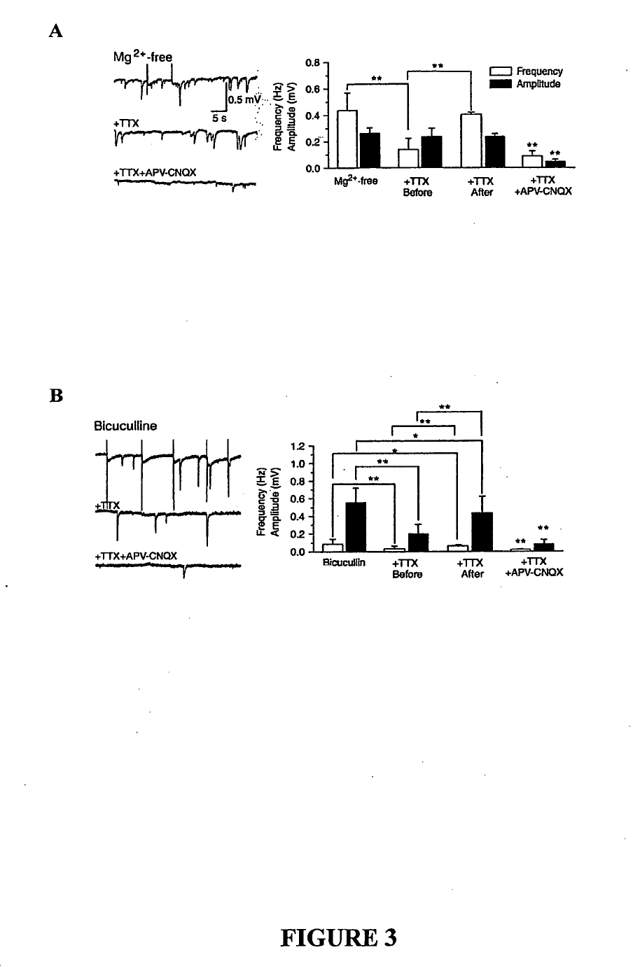

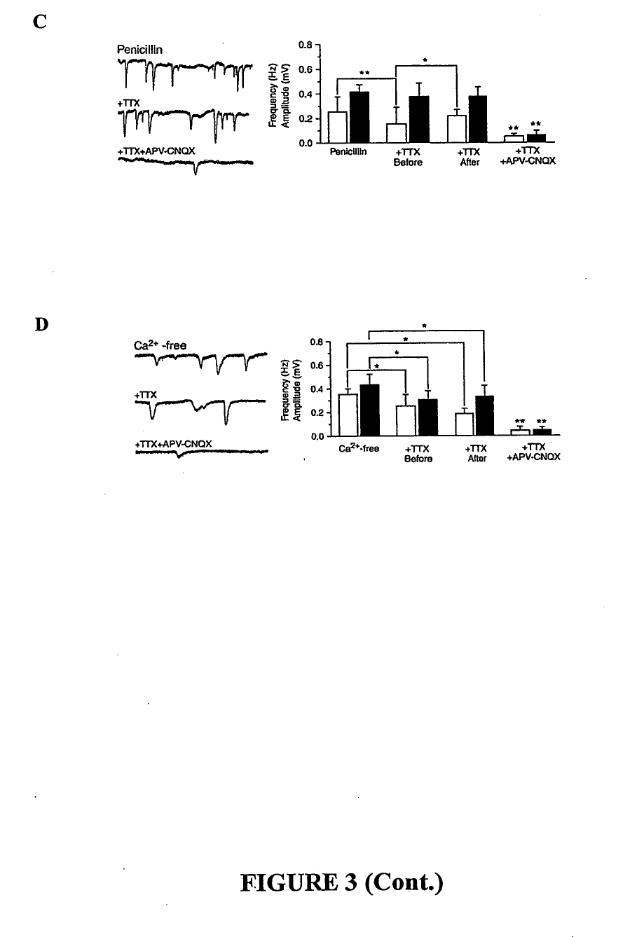

Seizures can be induced by a variety of inciting agents with apparently unrelated mechanisms of action. The traditionally defined mechanisms of epileptogenesis involve either the facilitation of excitatory synaptic activity, or the suppression of inhibitory transmission. To assess whether glutamate release from action potential-independent sources plays a role in experimental epilepsy, the dependence of PDSs upon TTX and glutamate receptor antagonists in several seizure models was analyzed. A common approach to induce hypersynchronous burst activity of large groups of neurons is to enhance excitatory synaptic activity by removing extracellular Mg²⁺. The epileptogenic action of Mg²⁺ depletion has been attributed to the activation of NMDA receptors at the resting membrane potential (Schuchmann et al., “Nitric Oxide Modulates Low-Mg2+-Induced Epileptiform Activity in Rat Hippocampal-Entorhinal Cortex Slices,” Neurobiol. Dis. 11:96-105 (2002), which is hereby incorporated by reference in its entirety). In accordance with earlier reports, Mg²⁺-free solution triggered repeated PDSs (FIG. 3A). These, however, were sustained in the presence of TTX despite complete elimination of action potentials, whereas APV/CNQX blocked more than 80% of PDSs (FIG. 3A). Thus, PDSs evoked by low extracellular Mg²⁺ appeared to result from glutamate released from action potential-independent sources, in addition to the removal of the Mg²⁺ block on NMDA receptors. Bicuculline and penicillin are potent convulsants in slice preparations (Schneiderman, J. H., “The Role of Long-Term Potentiation in Persistent Epileptiform Burst-Induced Hyperexcitability Following GABAA Receptor Blockade,” Neuroscience 81:1111-1122 (1997), which is hereby incorporated by reference in its entirety) and in animal models (Jones et al., “Effects of Bicuculline Methiodide on Fast (>200 Hz) Electrical Oscillations in Rat Somatosensory Cortex,” J. Neurophysiol. 88:1016-1125 (2002), which is hereby incorporated by reference in its entirety). The epileptogenic actions of bicuculline and penicillin have been ascribed to their antagonism of GABAA receptors (Schneiderman, J. H., “The Role of Long-Term Potentiation in Persistent Epileptiform Burst-Induced Hyperexcitability Following GABAA Receptor Blockade,” Neuroscience 81:1111-1122 (1997), which is hereby incorporated by reference in its entirety). Recordings, however, suggested that both bicuculline and penicillin, similar to 4-AP and Mg²⁺-free solution, triggered TTX-insensitive depolarization shifts resulting from extrasynaptic glutamate release and reception (FIGS. 3B-C). Simply lowering extracellular Ca²⁺ generates slow-wave and late-burst activity similar to seizures that occur in patients with hippocampal epilepsy (Perez-Velazquez et al., “Modulation of Gap Junctional Mechanisms During Calcium-Free Induced Field Burst Activity: A Possible Role for Electrotonic Coupling in Epileptogenesis,” J. Neurosci. 14:4308-4317 (1994), which is hereby incorporated by reference in its entirety). As in the other seizure models, Ca²⁺-free solution induced repeated TTX-insensitive depolarization shifts, resulting from activation of neuronal glutamate receptors (FIG. 3D). The effect of TTX added 10-15 min prior to inducing seizure activity vs addition of TTX 20 min later (when seizure activity was maximal) was compared. If TTX was added first, the frequency of PDSs were reduced in slices exposed to Mg²⁺-free solution, bicuculline, and penicillin, but not in slices incubated in Ca²⁺-free solution (FIG. 3). Similar to 4-AP (FIG. 2I), however, the major fraction of PDSs occurred independently of TTX addition before or after induction of seizure activity.

Thus, in all experimental models of seizure analyzed, including exposure to 4-AP, Mg²⁺-free solution, bicuculline, penicillin, and removal of extracellular Ca²⁺, PDSs were largely insensitive to TTX. Depending upon the model, TTX (and VGCC blockers) reduced the frequency of PDSs to 70-90% of total, demonstrating that the majority of PDSs was evoked by action potential-independent pathways. Another key observation was that glutamate is the principal mediator of TTX-insensitive PDS, because combined exposure to APV/CNQX/MCPG decreased the frequency of PDSs to 5-20%. The TTX- and APV/CNQX/MCPG-insensitive PDS might be elicited by other action potential-independent mechanisms, including gap junctions (Perez-Velazquez et al., “Modulation of Gap Junctional Mechanisms During Calcium-Free Induced Field Burst Activity: A Possible Role for Electrotonic Coupling in Epileptogenesis,” J. Neurosci. 14:4308-4317 (1994), which is hereby incorporated by reference in its entirety) and purinergic receptor activation possibly mediated by release of ATP by astrocytes (Cotrina et al., “Connexins Regulate Calcium Signaling by Controlling ATP Release,” Proc. Natl. Acad. Sci. USA 95:15735-15740 (1998); Cotrina et al., “ATP-Mediated Glia Signaling,” J. Neurosci. 20:2835-2844 (2000), which are hereby incorporated by reference in their entirety).

### Example 5

### TTX-Insensitive Astrocytic Ca²⁺ Signaling In Experimental Seizure Models

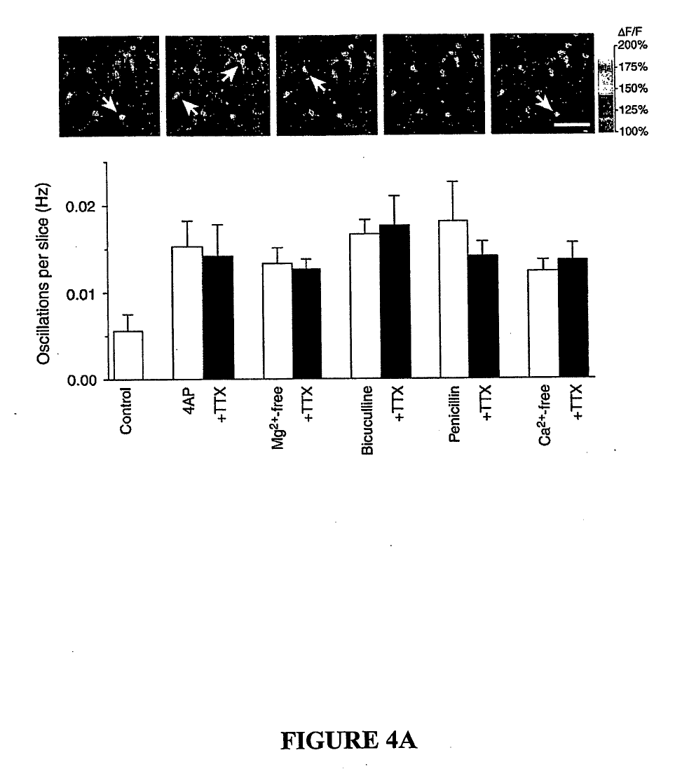

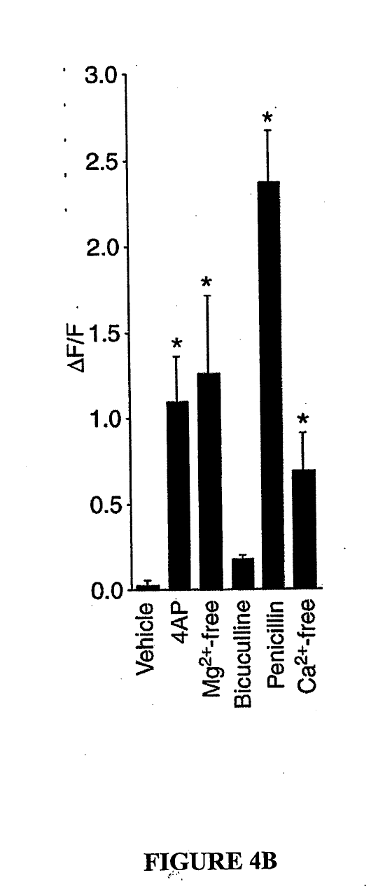

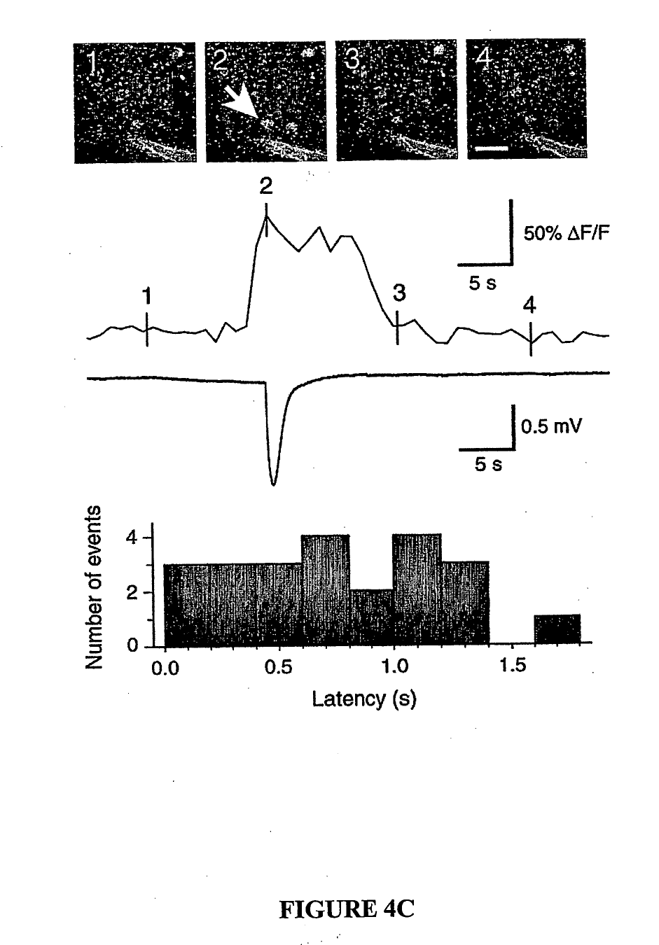

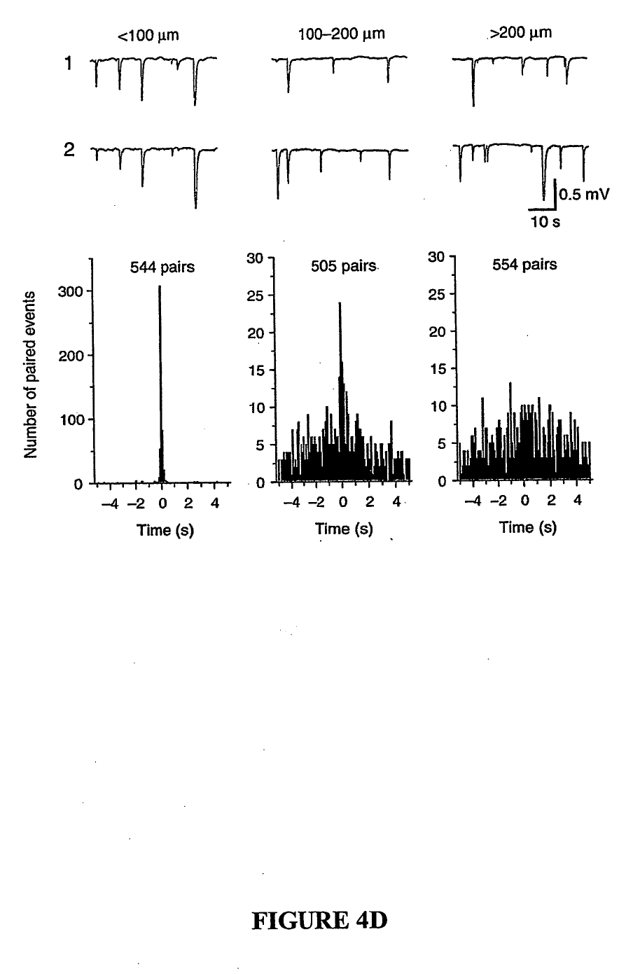

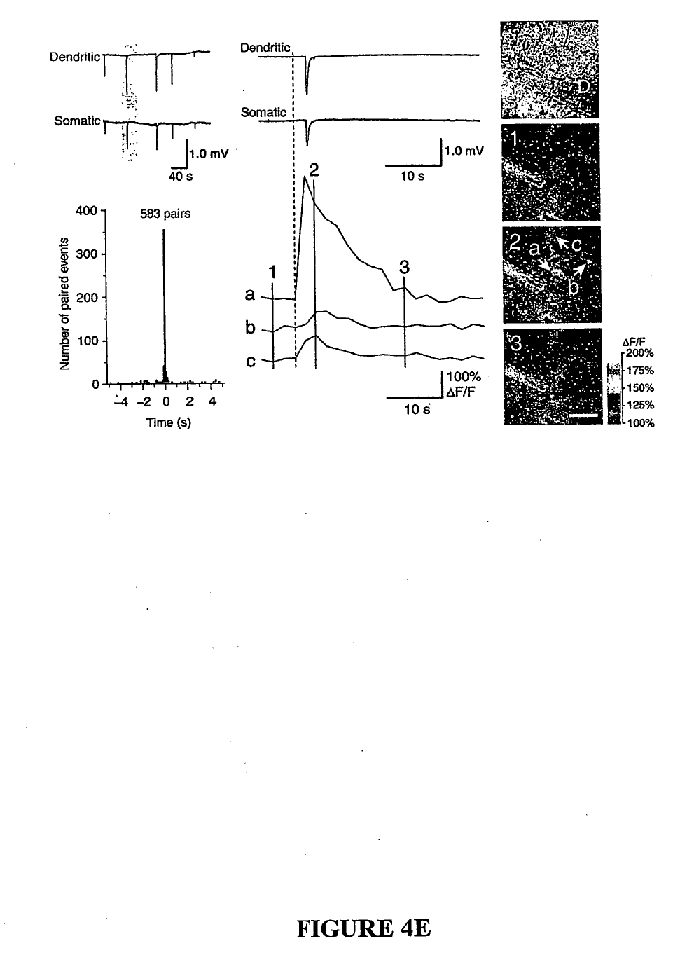

Recordings in hippocampal slices indicated that the cellular hallmark of epileptic discharge, PDSs, is caused by prolonged episodes (˜500 ms) of neuronal depolarization triggered by glutamate release from a non-synaptic source. Since a number of studies have documented that astrocytes can release glutamate in a Ca²⁺-dependent manner (Bezzi et al., “Prostaglandins Stimulate Calcium-Dependent Glutamate Release in Astrocytes,” Nature 391:281-285 (1998); Fellin et al., “Neuronal Synchrony Mediated by Astrocytic Glutamate Through Activation of Extrasynaptic NMDA Receptors,” Neuron 43:729-743 (2004); Angulo et al., “Glutamate Released from Glial Cells Synchronizes Neuronal Activity in the Hippocampus,” J. Neurosci. 24:6920-6927 (2004), which are hereby incorporated by reference in their entirety), whether activation of astrocytic Ca²⁺ signaling was a unifying feature of epileptogenesis was examined. Hippocampal slices were loaded with the Ca²⁺ indicator, fluo-4/AM and viewed by two-photon laser scanning microscopy. The preferential loading of fluorescent acetoxymethyl esters indicators by astrocytes has been extensively reported (Kang et al., “Astrocyte-Mediated Potentiation of Inhibitory Synaptic Transmission,” Nat. Neurosci. 1:683-692 (1998); Zonta et al., “Neuron-to-Astrocyte Signaling is Central to the Dynamic Control of Brain Microcirculation,” Nat. Neurosci. 6:43-50 (2003); Liu et al., “Astrocyte-Mediated Activation of Neuronal Kainate Receptors. Proc. Natl. Acad. Sci. USA 101:3172-3177 (2004), which are hereby incorporated by reference in their entirety). Bath application of 4-AP potently initiated astrocytic Ca²⁺ signaling expressed as infrequent Ca²⁺ oscillations (FIG. 4A). TTX did not reduce either the frequency or the amplitude of astrocytic Ca²⁺ oscillations, indicating that astrocytic activation was not an indirect effect of transmitters released during neuronal firing, but resulted from a direct action of 4-AP on astrocytes (paired t-test, P=0.4-0.8). In fact, 4-AP promptly induced Ca²⁺ signaling in cultured astrocytes, in the absence of co-cultured neurons, in accordance with previous publications (FIG. 4B) (Grimaldi et al., “Mobilization of Calcium from Intracellular Stores, Potentiation of Neurotransmitter-Induced Calcium Transients, and Capacitative Calcium Entry by 4-Aminopyridine,” J. Neurosci. 21:3135-3143 (2001)), which is hereby incorporated by reference in its entirety). Frequent TTX-insensitive Ca²⁺ oscillations were also observed in hippocampal slices exposed to Mg²⁺-free solution, bicuculline, penicillin, and Ca²⁺-free solution. Thus, all paradigms of experimental seizure studied potently triggered Ca²⁺ signaling of astrocytes in hippocampal slices in the absence of action potentials. Astrocytic Ca²⁺ signaling was expressed as slow oscillatory elevations of cytosolic Ca²⁺ lasting 10-60 s in individual astrocytes, but small groups of neighboring astrocytes also frequently displayed synchronized increases in Ca²⁺. Similar results were obtained in cultures of astrocytes (FIG. 4B) with the exception that bicuculline only weakly induced Ca²⁺ signaling. No explanation for the different response to bicuculline is apparent, but culturing of astrocytes is associated with major alterations of both morphology and receptor expression (Ransom et al., “New Roles for Astrocytes (Stars at Last),” Trends Neurosci. 26:520-522 (2003), which is hereby incorporated by reference in its entirety). The combination of Ca²⁺ imaging with field potential recordings was done to establish the temporal connection between the two events (FIG. 4C). When the field electrode was placed in close proximity to the astrocytic cell body, at an average distance of 22±2 μm (range 10 to 30 μm, n=23, 7 slices), it was found that oscillatory increases in astrocytic Ca²⁺ were linked to a negative shift in field potential (0.38±0.06 mV, range 0.2 to 1.17 mV, 23 of a total of 45 spontaneous astrocytic Ca²⁺ increases in a total of 8 slices). Interestingly, the oscillatory increase in astrocytic Ca²⁺ always preceded the drop in field potential. The average delay between the onset of astrocytic Ca²⁺ increase to the onset of a decrease in field potential was 0.38±0.06 s (range 0.05 to 1.69 s) (FIG. 4C).

Astrocytes within the cortex and hippocampus are organized in essentially non-overlapping microdomains with an average diameter of 40-70 μm, reviewed in Nedergaard et al., “New Roles for Astrocytes: Redefining the Functional Architecture of the Brain,” Trends Neurosci. 26:523-530 (2003), which is hereby incorporated by reference in its entirety). Since Ca²⁺ oscillations are restricted to 1-3 neighboring astrocytes, it is expected that the PDSs are limited to small (<50-200 μm) regions. To establish the spatial territories of PDSs, recording with two field electrodes in the stratum radiatum of CA1 was performed (FIG. 4D). Paired events were arbitrarily defined as negative shifts that occurred within a 5 s window at both electrode sites. If the electrodes were positioned <100 μm apart, 56% of the paroxysmal depolarizations were temporally synchronized (307 of 544 events occurred within 100 ms of each other). When the electrodes were placed at a distance ranging from 100-200 μm, 4.8% of the paired events (24 of 505) occurred within the time window of 100 ms, whereas only 1.4% of events (8 of 554) were synchronized if the electrodes were greater than 200 μm apart. Typically, the amplitude of the field potential deflections varied as a function of time and from event to event. Similarly, if the two electrodes were placed in the stratum radiatum and the stratum pyramidale, respectively, simultaneous depolarization events were observed in 356 of 583 pairs (61%) (FIG. 4E). Interestingly, the PDSs in the stratum pyramidale were preceded by Ca²⁺ increases in astrocytes, similar to PDSs recorded in the stratum radiatum (FIG. 4C).

Taken together, these observations demonstrate that astrocytic Ca²⁺ signaling is evoked in 5 different models of acute seizure. In all paradigms studied, astrocytic Ca²⁺ signaling was insensitive to TTX indicating direct stimulation of astrocytes, rather than a secondary response to neuronal bursting activity. Furthermore, PDSs were spatially restricted to a few hundred micrometers and increments in cytosolic Ca²⁺ of astrocytes always preceded PDSs by in the stratum radiatum.

### Example 6

### Photolysis of Caged Ca²⁺ in Astrocytes Triggers Paroxysmal Depolarization Shifts

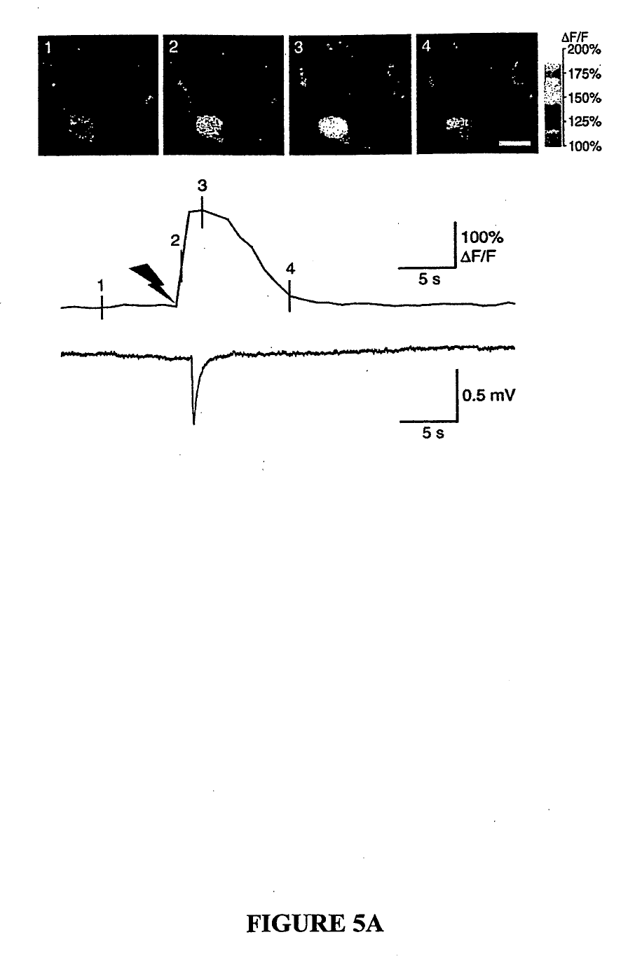

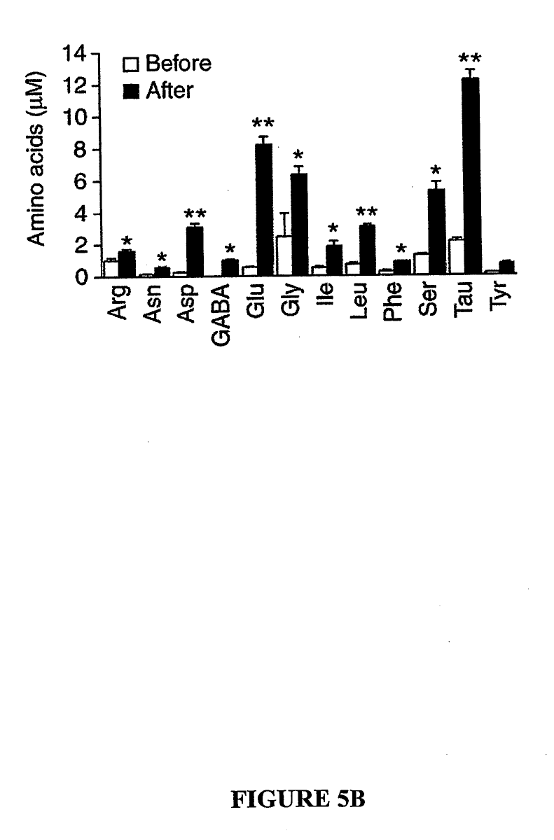

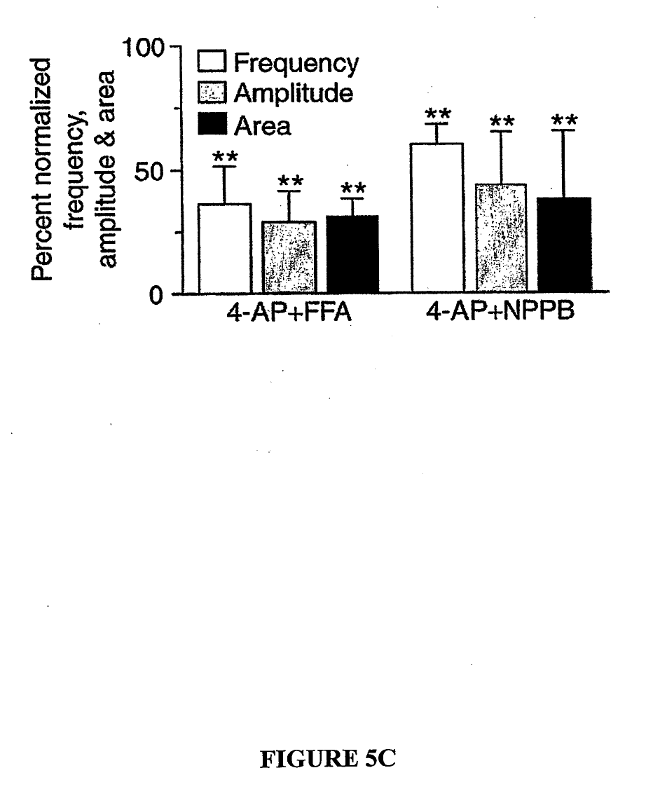

To demonstrate that astrocytic activation is not only correlated with, but sufficient for generation of negative depolarization shifts, photo release of caged Ca²⁺ (NP-EGTA) in astrocytes was performed (FIG. 5A) (Liu et al., “Astrocyte-Mediated Activation of Neuronal Kainate Receptors. Proc. Natl. Acad. Sci. USA 101:3172-3177 (2004), which is hereby incorporated by reference in its entirety). Increases in astrocytic Ca²⁺ evoked by uncaging of NP-EGTA triggered in 8 of 12 experiments a PDS, whereas UV-flash in an identical fashion of slices not loaded with NP-EGTA failed to increase astrocytic Ca²⁺ concentration or to evoke PDSs (n=15) (FIG. 5A). Targeting neurons with the UV beam also failed to evoke PDSs (n=7). This set of observations indicates that increases in astrocytic Ca²⁺ are sufficient to induce local depolarization shifts.

One of the characteristics of Ca²⁺-dependent astrocytic glutamate release is that other amino acids, including aspartate, glutamine, and taurine also are released (Jeremic et al., “ATP Stimulates Calcium-Dependent Glutamate Release from Cultured Astrocytes,” J. Neurochem. 77:664-675 (2001); Nedergaard et al., “Beyond the Role of Glutamate as a Neurotransmitter,” Nat. Rev. Neurosci. 3:748-755 (2002), which are hereby incorporated by reference in their entirety). These amino acids exit through volume sensitive channels (VSC) expressed by astrocytes, whereas other amino acids, including asparagine, isoleucine, leucine, phenylalanine and tyrosine, are released to a lesser extent. To test the idea that astrocytes release glutamate during epileptic seizures, a microdialysis probe with a built-in electrode for EEG recording (Obrenovitch et al., “Evidence Disputing the Link Between Seizure Activity and High Extracellular Glutamate,” J. Neurochem. 66:2446-2454 (1996), which is hereby incorporated by reference in its entirety), was implanted in the hippocampus and perfused with artificial cerebrospinal fluid (ACSF) containing 4-AP. The basal extracellular concentration of glutamate was low in accordance with earlier reports (0.5-1.5 μM) (Mena et al., “In vivo Glutamine Hydrolysis in the Formation of Extracellular Glutamate in the Injured Rat Brain,” J. Neurosci. Res. 60:632-641 (2000), which is hereby incorporated by reference in its entirety), but increased to 6-10 μM approximately 10 min after addition of 4-AP. Consistent with the idea that glutamate is released by astrocytes, a 3-8 fold increase in release of amino acid osmolytes, including glutamate, aspartate, glutamine, and taurine, was observed (FIG. 5B). This profile of amino acid release was very similar to the profile of amino acid release triggered by Ca²⁺ signaling in cultured astrocytes, with the exception that the concentration of non-osmolyte amino acids doubled during seizure activity. The shrinkage of the extracellular space that occurs during seizure activity has previously been reported to cause an artificial increase in the concentration of compounds collected by microdialysis (Benveniste et al., “Microdialysis—Theory and Application,” Prog. Neurobiol: 35:195-215 (1990), which is hereby incorporated by reference in its entirety).

Given that photolysis experiments and HPLC analysis indicated that astrocytes contribute to elevations in extrasynaptic glutamate in epileptic tissue, it was predicted that compounds that reduce astrocytic glutamate release would suppress epileptiform activity. Based on culture experiments, it has been documented that anion channel inhibitors, including 5-nitro-2-(3-phenylpropylamino) benzoic acid (“NPPB”) and flufenamic acid (“FFA”), reduce glutamate release from astrocytes (Nedergaard et al., “Beyond the Role of Glutamate as a Neurotransmitter,” Nat. Rev. Neurosci. 3:748-755 (2002), which is hereby incorporated by reference in its entirety). To evaluate the effect of anion channel inhibition upon epileptic discharges, FFA or NPPB were bath applied to hippocampal slices exhibiting 4-AP induced seizures. Both NPPB and FFA markedly reduced the frequency and amplitude of PDSs (FIG. 5C).

Combined, these observations indicate: 1) that targeting astrocytes by photolysis of caged Ca²⁺ triggered PDSs, whereas similar stimulation of neurons had no effect upon the field potential; 2) that the footprint of amino acids released during 4-AP induced seizures was similar to Ca²⁺-dependent amino acids released from cultured astrocytes, and; 3) that anion channel inhibitors reduce the frequency and amplitude of PDSs. Together, these findings support the idea that astrocytes contribute to action potential-independent glutamate release in 4-AP evoked seizures.

### Example 7

### Suppression of Astrocytic Ca²⁺ Signaling by Anti-Epileptic Drugs

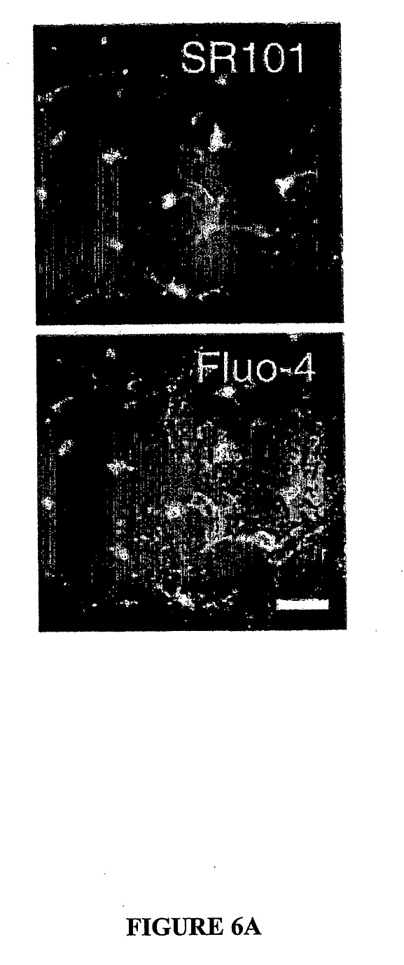

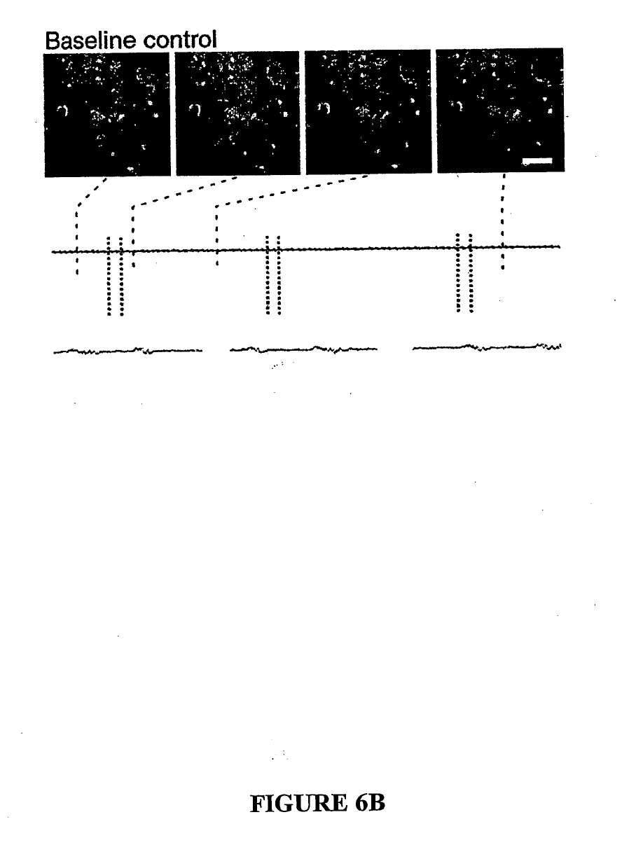

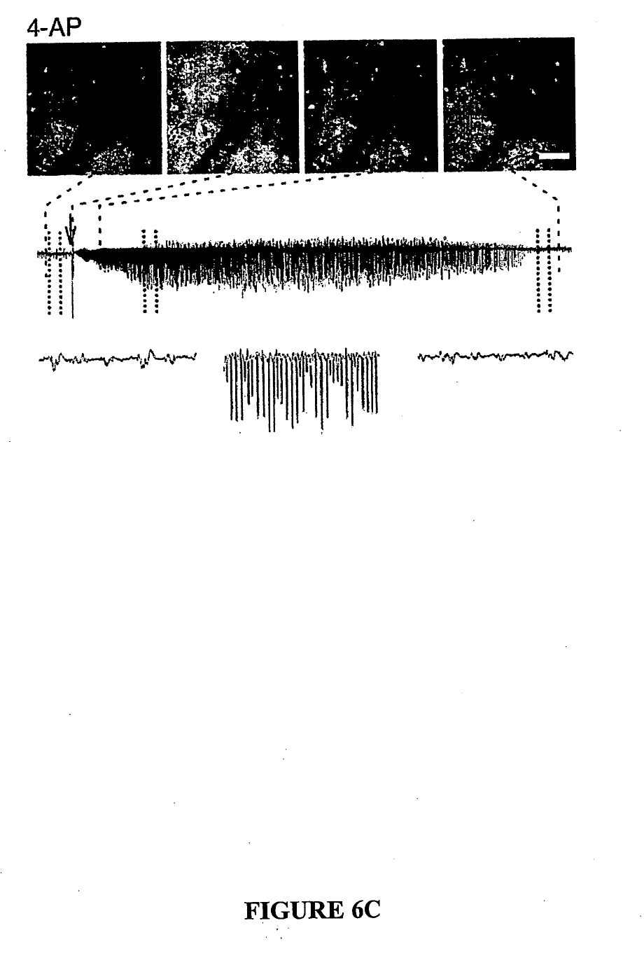

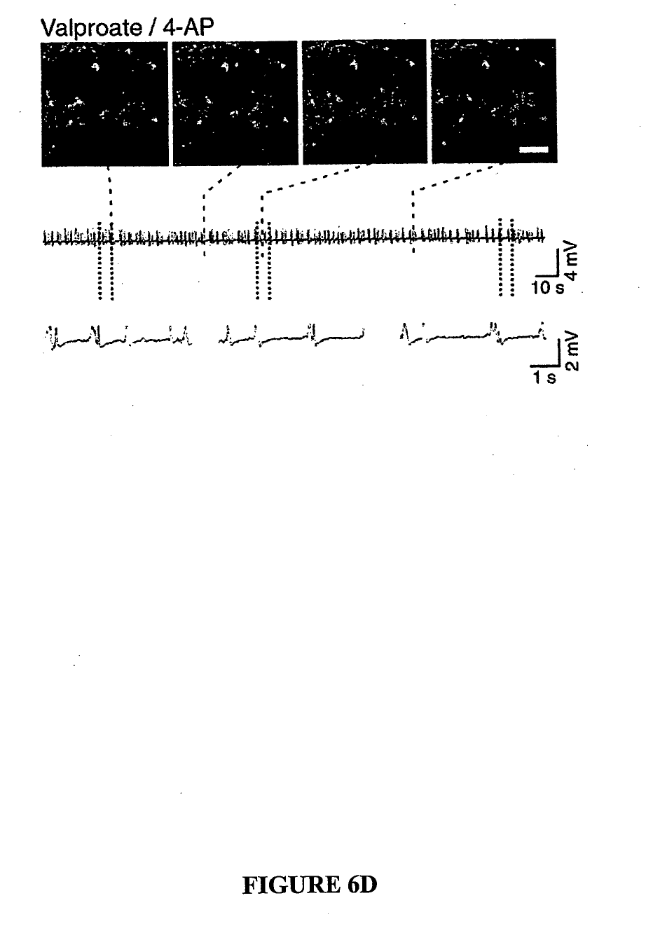

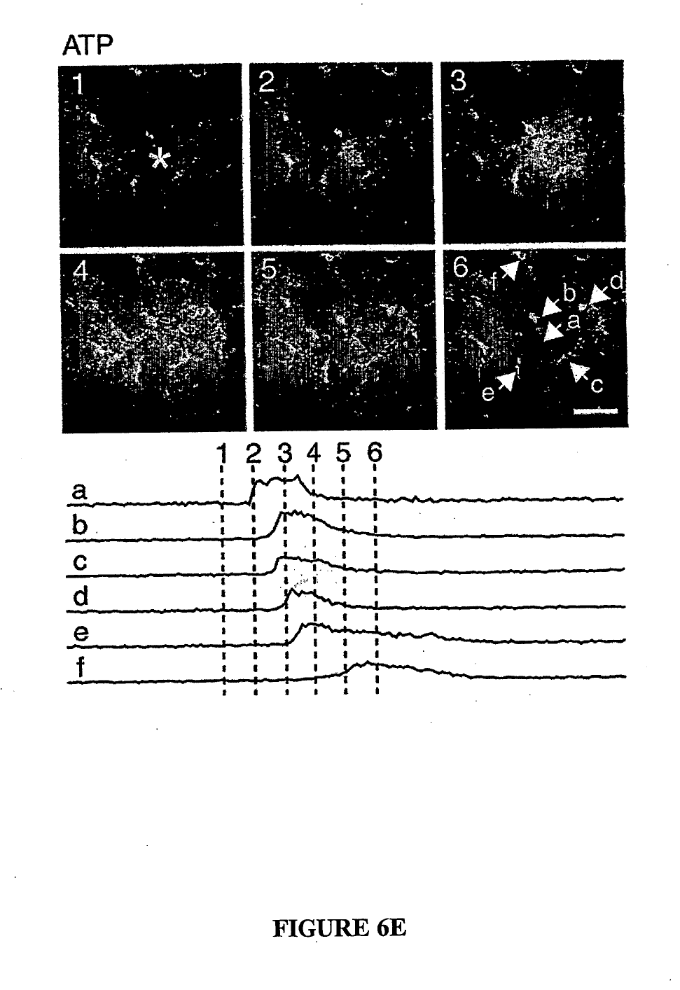

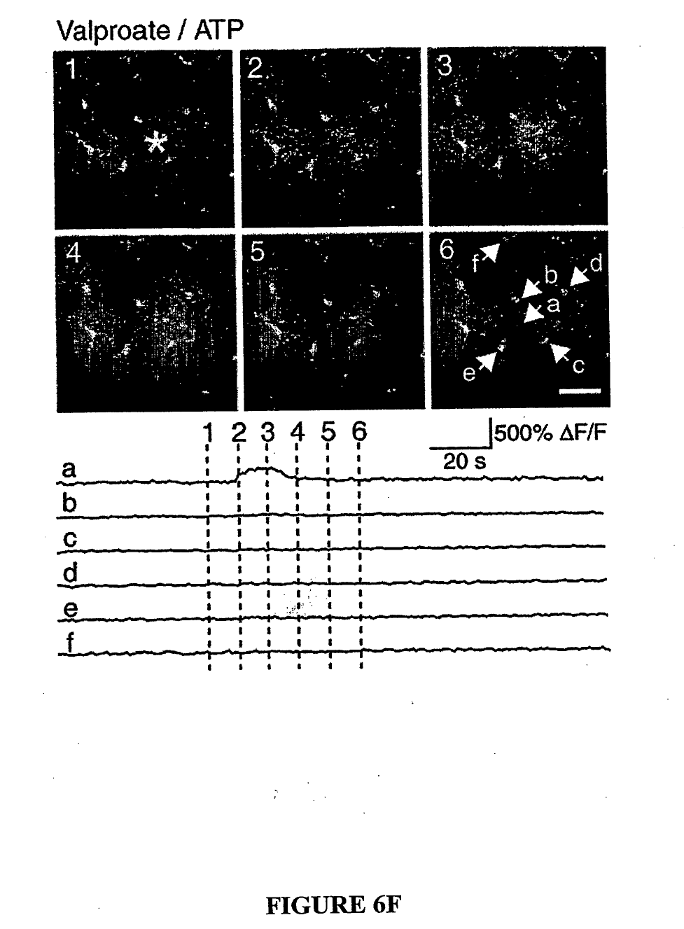

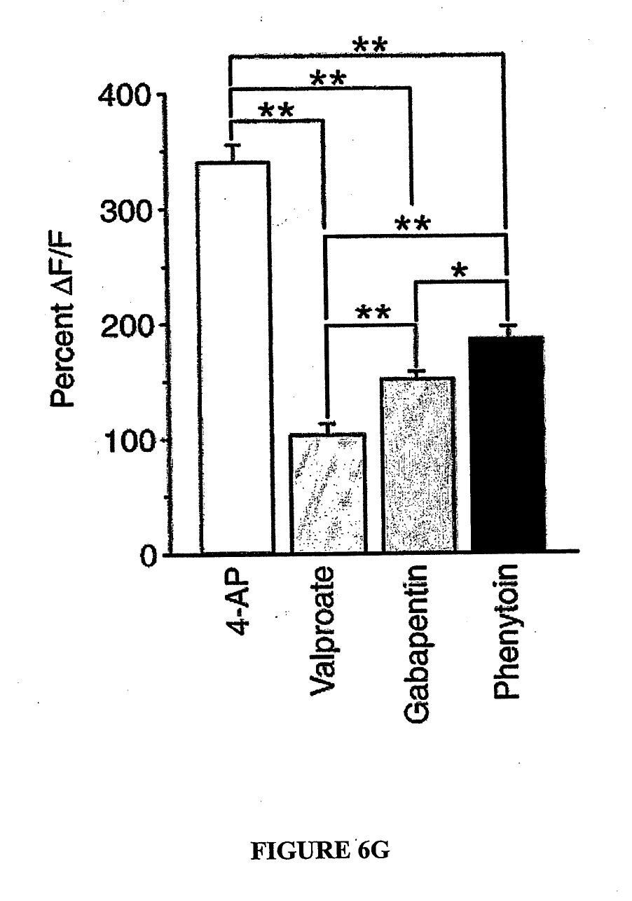

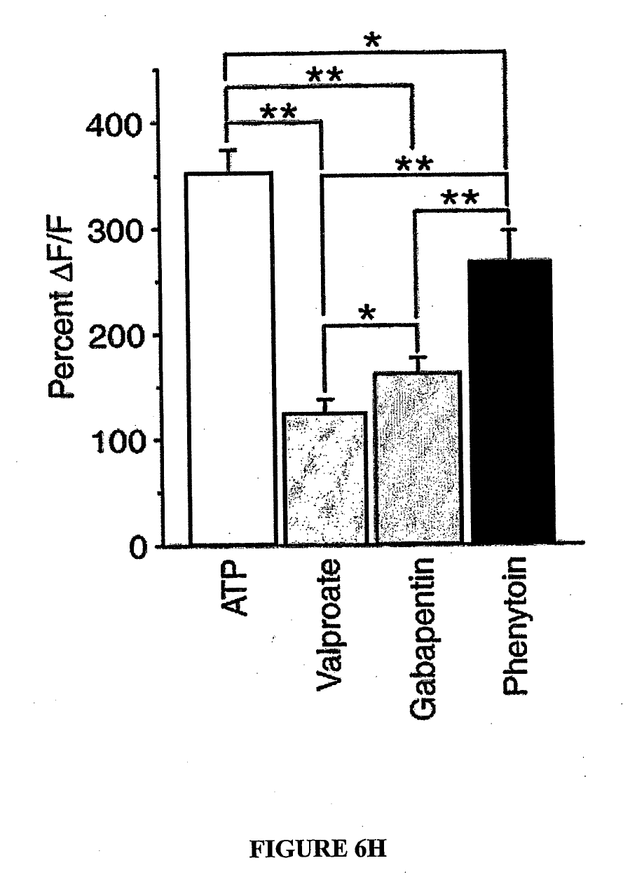

To test the importance of astrocytic activation in generation of seizures in live animals, two-photon imaging of Ca²⁺ signaling in the exposed cortex of adult mice was used. The primary somatosensory cortex was loaded with fluo-4/AM prior to imaging. In initial experiments, Fluo-4/AM was loaded concomitant with the astrocyte specific marker Sulforhodamine 101 (Nimmerjahn et al., “Sulforhodamine 101 as a Specific Marker of Astroglia in the Neocortex In vivo,” Nature Methods 1:1-7 (2004), which is hereby incorporated by reference in its entirety). Fluo-4 and Sulforhodamine 101 were co-localized, indicating that fluo-4 is preferentially taken up by astrocytes in live exposed cortex as previously reported (FIG. 6A) (Hirase et al., “Capillary Level Imaging of Local Cerebral Blood Flow in Bicuculline-Induced Epileptic foci,” Neuroscience 128:209-216 (2004), which is hereby incorporated by reference in its entirety). 4-AP was delivered locally by an electrode used for recording of the field potential. Application of 4-AP triggered propagating Ca²⁺ waves and repeated oscillatory increases in Ca²⁺. In addition, astrocytes displayed Ca²⁺ signaling in conjunction with the spontaneous seizure activity that occurred 5-30 min after application of 4-AP (FIGS. 6B and 6C). Only these late events were further analyzed. Interestingly, astrocytic Ca²⁺ signaling preceded bursting activity (FIG. 6C). A total of 31 epileptic activities in 5 animals, where 19 were preceded by astrocytic Ca²⁺ increases in the area of the recordings, were recorded. The increases in astrocytic Ca²⁺ occurred 4.7±2.8 sec (Mean±SD, n=19) prior to seizure activity and were characterized by a widespread increase in Ca²⁺ across multiple astrocytes. Only two episodes of spontaneous astrocytic Ca²⁺ increases (2 of 21 total events) were not linked to epileptiform activity. Opposite, 19 of 31 seizure-like neuronal burstings were preceded by astrocytic Ca²⁺ signaling, whereas 10 seizure-like events occurred without an increase in astrocytic Ca²⁺. Valproate reduced both the amplitude of neuronal discharges and astrocytic responses to 4-AP (FIG. 6D). 4-AP-induced Ca²⁺ signaling was reduced by 69.7% in animals treated with valproate, by 55.6% in animals with gabapentin, and by 45.5% in animals with phenyloin (FIG. 6G). Thus, three commonly used anti-epileptic drugs all depressed astrocytic Ca²⁺ signaling triggered by 4-AP. Since previous work has established that astrocytic Ca²⁺ signaling is activated during seizure activity (Hirase et al., “Capillary Level Imaging of Local Cerebral Blood Flow in Bicuculline-Induced Epileptic foci,” Neuroscience 128:209-216 (2004); Tashiro et al., “Calcium Oscillations in Neocortical Astrocytes under Epileptiform Conditions,” J. Neurobiol. 50:45-55 (2002), which are hereby incorporated by reference in their entirety), the inhibition of astrocytic Ca²⁺ signaling likely reflects that the anti-epileptics reduced the neuronal activity. To test the alternative idea, that valproate, gabapentin, and phenyloin, directly targeted astrocytes and by suppression of their ability to transmit Ca²⁺ signaling reduced epileptiform activity, Ca²⁺ waves by iontophoretic application of ATP was evoked. In control animals, ATP triggered astrocytic Ca²⁺ waves, which propagated and spread beyond the field of view, as shown in FIG. 6E. In animals pretreated with valproate, gabapentin, and phenyloin, Ca²⁺ wave propagation was significantly decreased (FIGS. 6F and 6H). Valproate depressed Ca²⁺ signaling by 64.9%, gabapentin by 53.8%, whereas phenyloin was least efficient (23.8%). Thus, all anti-epileptics tested directly suppressed astrocytic Ca²⁺ signaling evoked by purinergic receptor stimulation in control non-epileptic mice.

### Example 8

### Models of Chronic Epilepsy

Post-traumatice epilepsy induced by intracortical Iron Injection: Adult mice (2 months) can be anesthetized using a ketamine (100 mg/kg) and xylazine (25 mg/kg) mixture and positioned in a stereotaxic frame. Following a small craniotomy and opening of the dura, 1.0 μl of 100 mM ferrous chloride solution can be injected into sensorimotor cortex (1.5-2.0 mm posterior to bregma, 1.0-1.5 mm lateral to midline, and 0.5-1.0 mm below the cortical surface) at a rate of 1.0 μl/min using a microprocessor controlled syringe pump (Model 210, Stoelting Co. IL, USA) (Willmore et al., “Chronic Focal Epileptiform Discharges Induced by Injection of Iron into Rat and Cat Cortex”, Science 200:1501-1503, (1978)). Electroencephalography can be obtained at 2 months after intracortical injections of ferric chloride (Shah et al., “Seizure-induced Plasticiy of H Channels in Entorhinal Corical Layer III Pyramidal Neurons”, Neuron 44:495-508 (2004)). More than 90% animal will develop spontaneous epileptiform EEG-activity after intracortical injection of ferrous chloride.

Genetic epilepsy: Genetic epilepsy mice-tottering mice (B6.D2-cacna1a^{tg}/J), which are genetically predisposed to epilepsy due to a mutation in the voltage gated calcium channel subunit α1A (Tg⁻) (Fletcher et al., “Absence Epilepsy in Tottering Mutant mice is Associated with Calcium Channel Defects”, Cell 87:607-617 (1996)), were obtained from the Jackson Laboratory (JAX #000544). Onset of seizures occurs usually 3-4 weeks of age and symptoms persist throughout life. The Tottering mouse has a characteristic wobbly gait and display bilaterally synchronous spike-wakes in EEG recordings of 1-10 seconds in duration many times during a day. Stereotypic partial motor seizures with abnormal ECG activity also occur once or twice a day and are usually 20-30 minutes in duration.

### Example 9

### Paroxysmal Depolarization Shifts Preceded Epileptiform Bursting Activities

Paroxysmal depolarization shifts are abnormal prolonged depolarizations with repetitive spiking and are reflected as interictal discharges in the electroencephalogram (Heinemann et al., “Contribution of Astrocytes to Seizure Activity”, Adv. Neurol. 79:583-590 (1999)). Here, it has been demonstrated that glutamate released from astrocytes can trigger paroxysmal depolarization shifts in several models of acute experimental seizure (Tian et al., “An Astrocytic Basis of Epilepsy” Nature Med. 11:973-981 (2005)). A unifying feature of seizure activity was its consistent association with antecedent astrocytic Ca²⁺ signaling. Oscillatory, TTX-insensitive increases in astrocytic Ca²⁺ preceded or occurred concomitantly with paroxysmal depolarization shifts, and targeting astrocytes by photolysis of caged Ca²⁺ evoked paroxysmal depolarization shifts. Furthermore, several anti-epileptic agents, including valproate, gabapentin, and phenyloin, potently reduced astrocytic Ca²⁺ signaling detected by 2-photon imaging in live animals. This suggests that pathologic activation of astrocytes may play a central role in the genesis of epilepsy, as well in the pathways targeted by current anti-epileptics.

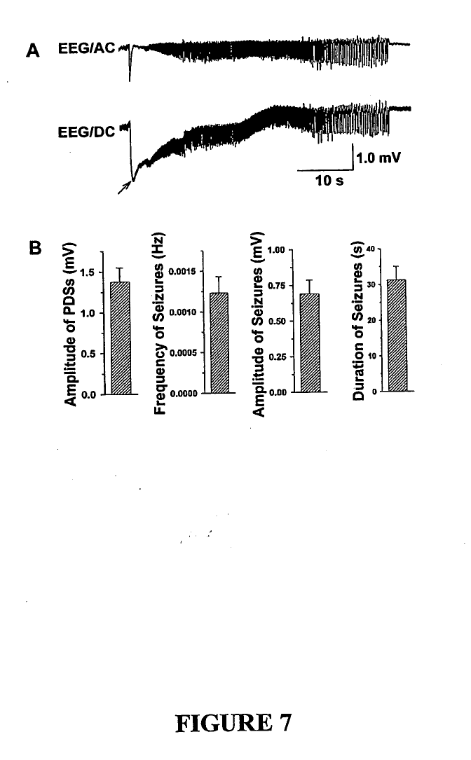

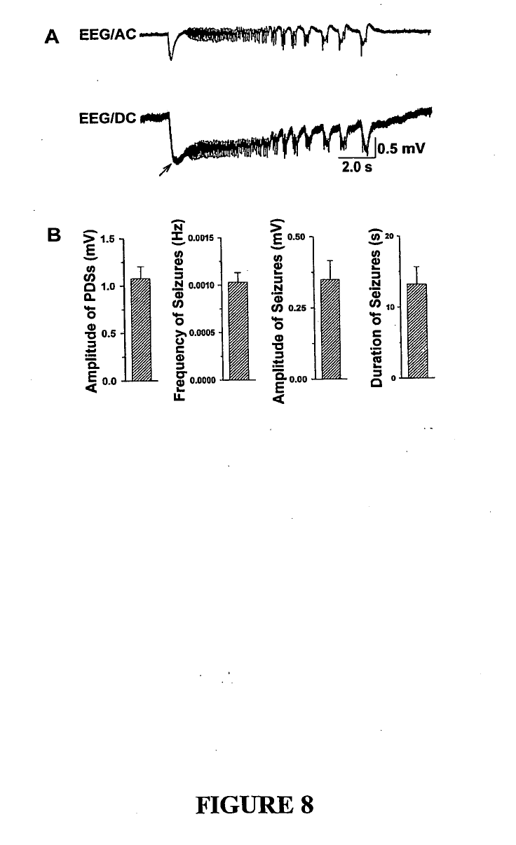

It has been observed that paroxysmal depolarization shifts preceded epileptiform EEG in mice with intracortical injection of ferric chloride 2 months prior (FIG. 7) and in genetic epilepsy mice (B6.D2-Cacnala^{tg}/J) (FIG. 8). This observation confirmed and extended in vitro observations according to the present invention (e.g. 4-AP, bicuculline, penicilline) to in vivo models of chronic epilepsy.

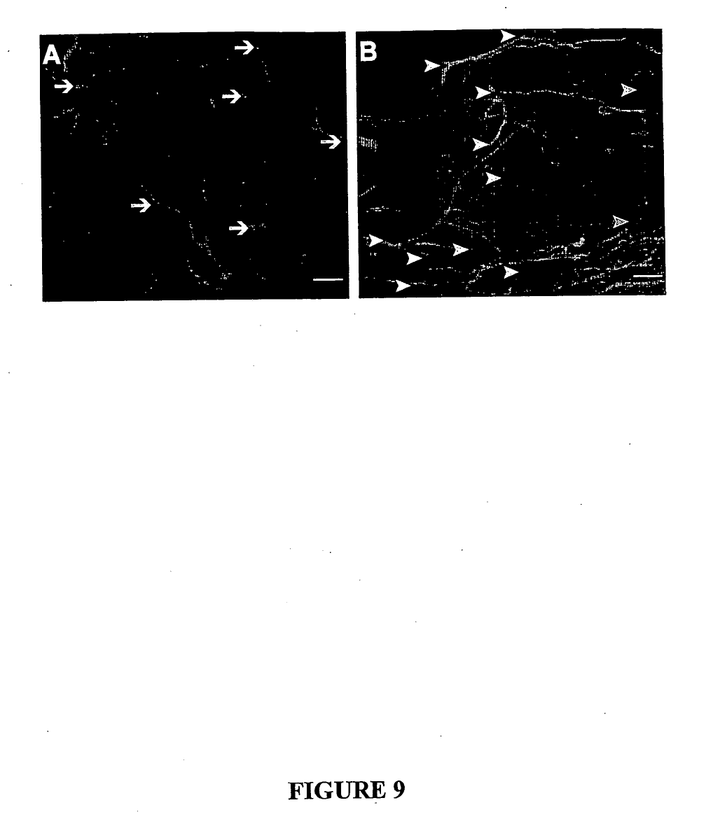

It has also been observed that Cx43 formed large plaques in the cortex of mice with intracortical injection of ferric chloride (FIG. 9B), whereas Cx43 in the cortex of control mice distributed evenly (FIG. 9A). Much denser GFAP expression exists in the tissues of mice with intracortical injection of ferric chloride, evident in FIG. 9B.

According to the present invention, prolonged episodes of neuronal depolarization evoked by astrocytic glutamate release contribute to epileptiform discharges. Synchronized population spikes are key concomitants to seizure. Prior studies have demonstrated that multisynaptic excitatory pathways can trigger synchronized burst activity in picrotoxin-induced seizure activity (Miles et al., “Single Neurones Can Initiate Synchronized Population Discharge in the Hippocampus,” Nature 306:371-373 (1983), which is hereby incorporated by reference in its entirety), whereas other evidence has been presented for roles of both recurrent inhibition and gap junction coupling (Perez-Velazquez et al., “Modulation of Gap Junctional Mechanisms During Calcium-Free Induced Field Burst Activity: A Possible Role for Electrotonic Coupling in Epileptogenesis,” J. Neurosci. 14:4308-4317 (1994), which is hereby incorporated by reference in its entirety).

According to the present invention, additional mechanism exists indicating that an action potential-independent source of glutamate can trigger local depolarization events and synchronized bursting activity. That other cells, including neurons, contribute to extrasynaptic glutamate release cannot be excluded, but several observations point to astrocytes as the primary source. First, the existence of a Ca²⁺-dependent mechanism of astrocytic glutamate release has been documented by several groups (Haydon, P. G., “GLIA: Listening and Talking to the Synapse,” Nat. Rev. Neurosci. 2:185-193 (2001), which is hereby incorporated by reference in its entirety). Second, photolysis of caged Ca²⁺ in astrocytes was sufficient to trigger PDSs, as shown in FIG. 5A. Third, astrocytic Ca²⁺ signaling was triggered in all models of seizure studied, as shown in FIG. 4A. Fourth, glutamate was not released in isolation, but was joined by the release of several amino acids present in the cytosol of astrocytes, including aspartate and taurine, as depicted in FIG. 5B. Fifth, all conventional anti-epileptics tested suppressed astrocytic Ca²⁺ signaling following systemic administration (FIG. 6G).

According to the present invention, 70-90% of PDSs were TTX-insensitive, indicating that a non-synaptic mechanism played a predominant role in generating seizure activity in the 5 models of experimental epilepsy studied. This observation does not exclude that astrocytes may play a role in seizure activity that originate in neurons. Astrocytes may amplify, maintain, and expand neurogenic seizure activity. Excessive neuronal firing is associated with marked alterations in the composition of the extracellular ions, most notably an increase in K⁺ and a reduction of Ca²⁺ (Heinemann et al., “Extracellular Calcium and Potassium Concentration Changes in Chronic Epileptic Brain Tissue,” Adv. Neurol. 44:641-461 (1986), which is hereby incorporated by reference in its entirety). Lowering of extracellular Ca²⁺ potently elicits astrocytic Ca²⁺ signaling (Stout et al., “Modulation of Intercellular Calcium Signaling in Astrocytes by Extracellular Calcium and Magnesium,: Glia 43:265-273 (2003), which is hereby incorporated by reference in its entirety) and glutamate release (Ye et al., “Functional Hemichannels in Astrocytes: A Novel Mechanism of Glutamate Release,” J. Neurosci. 23:3588-3596 (2003), which is hereby incorporated by reference in its entirety), and secondary engagement of astrocytes may convert an otherwise self-limited episode of intense neuronal firing into a seizure focus. It is also possible that spillover of glutamate from excitatory synapses contributes to activation of astrocytic Ca²⁺ signaling by binding to mGluR (Zonta et al., “Neuron-to-Astrocyte Signaling is Central to the Dynamic Control of Brain Microcirculation,” Nat. Neurosci. 6:43-50 (2003), which is hereby incorporated by reference in its entirety). Thus, astrocytes may initially be activated by excessive neuronal activity, but once activated, neuronal firing may no longer be required for continued activity of astrocytes, and thereby for maintenance and propagation of abnormal electrical activity.

Similar action potential-independent mechanisms may underlie local expansion of a seizure focus. Lowering of extracellular Ca²⁺ triggers propagation of astrocytic Ca²⁺ waves that spread into adjacent tissue (Arcuino et al., “Intercellular Calcium Signaling Mediated by Point-Source Burst Release of ATP,” Proc. Natl. Acad. Sci. USA 99:9840-9845 (2002), which is hereby incorporated by reference in its entirety). Long-distance astrocytic Ca²⁺ waves excite neurons along their path by release of glutamate (Nedergaard et al., “Beyond the Role of Glutamate as a Neurotransmitter,” Nat. Rev. Neurosci. 3:748-755 (2002), which is hereby incorporated by reference in its entirety). In turn, neuronal activity lowers extracellular Ca²⁺ resulting in activation of astrocytes in increasing distances from the seizure focus (Bikson et al., “Modulation of Burst Frequency, Duration, and Amplitude in the Zero-Ca(2+) Model of Epileptiform Activity,” J. Neurophysiol. 82:2262-2270 (1999), which is hereby incorporated by reference in its entirety). Thus, a cascade of events in which astrocytic Ca²⁺ signaling plays a key role may cause conversion of normal brain tissue remote from the center of seizure initiation into an epileptic focus.

The new observation reported here is that astrocytic activation can directly trigger seizure activity and that epilepsy thereby, at least in part, may originate in astrocytes.

It is proposed that seizure activity may have an astrocytic basis, in addition to the well-established neurogenic mechanisms. The primary argument for existence of an astrocytic basis for seizure is that the larger fraction (70-90%) of PDSs was TTX-insensitive in five experimental models of seizure studied (FIGS. 1-3). The new observation, according to the present invention, is that astrocytic glutamate release constitutes a mechanism for generation of PDS, and thereby for hypersynchronous neuronal firing. Accepting that seizure activity can originate from both astrocytes and neurons, it is also of importance to acknowledge that both astrocytes and neurons may contribute to the maintenance and spread of seizure activity. Even in gliogenic-induced seizures, excessive neuronal activity is associated with increases in interstitial K⁺, decreases in Ca²⁺, and additional glutamate release. High K⁺, low Ca²⁺, and glutamate (Zonta et al., “Neuron-to-Astrocyte Signaling is Central to the Dynamic Control of Brain Microcirculation,” Nat. Neurosci. 6:43-50 (2003); Stout et al., “Modulation of Intercellular Calcium Signaling in Astrocytes by Extracellular Calcium and Magnesium, Glia 43:265-273 (2003); Carmignoto et al., “On the Role of Voltage-Dependent Calcium Channels in Calcium Signaling of Astrocytes In situ,” J. Neurosci. 18:4637-4645 (1998), which are hereby incorporated by reference in their entirety) are all potent triggers of astrocytic Ca²⁺ signaling and may be independent of etiology of the seizure due to secondary astrocytic activation. Synaptic mechanisms can on the other hand also amplify or generalize a local seizure focus (Meldrum, B. S., “Update on the Mechanism of Action of Antiepileptic Drugs,” Epilepsia 37 (Suppl.):6, S4-11 (1996), which is hereby incorporated by reference in its entirety). Because trans-synaptic spread of seizure activity is driven by synaptic input, it is likely that TTX-sensitive seizures are not preceded by astrocytic Ca²⁺ increases or PDS. Consistent with this idea, 90% of Ca²⁺ increments in astrocytes (19 of 21) was followed by a seizure-like event in animals exposed to 4-AP, whereas only 61% (19 of 31) of the seizure events was preceded by increases in astrocytic Ca²⁺ (FIGS. 6C and 6G).

Existing drugs available for treatment of epilepsy fall into three categories. Na⁺ channel blockers attenuate high-frequency firing by reducing the amplitude and rate of rise of action potentials. GABA receptor agonists mimic the action of GABA, thereby increasing inhibitory synaptic transmission. Lastly, glutamate receptor antagonists block ionotopic glutamate receptors thereby reducing excitatory synaptic transmission (Rogawski et al., “The Neurobiology of Antiepileptic Drugs for the Treatment of Nonepileptic Conditions,” Nat. Med. 10:685-692 (2004), which is hereby incorporated by reference in its entirety). The downside of these drugs is that the therapeutic mechanisms of action also suppress normal neural activity. Valproate, gabapentin, and phenyloin all reduced astrocytic Ca²⁺ signaling in animals exposed to 4-AP. Even more intriguing, valproate, gabapentin, and phenyloin directly depressed astrocytic Ca²⁺ signaling evoked by purinergic receptor activation, demonstrating a direct effect on the ability of astrocytes to mobilize Ca²⁺ and/or transmit intercellular Ca²⁺ signaling. Thus, the anticonvulsive activity of valproate, gabapentin, and phenyloin, may be mediated by directly depressing astrocytic activity. Since the results of the above experiments suggest that epileptic discharges are secondary to glial pathology, astrocytes may represent a promising new target for epileptogenic interventions. Pharmacotherapy directed specifically at suppressing glial Ca²⁺ signaling or decreasing TTX-insensitive glutamate release may achieve seizure control, without the suppression of neural transmission associated with current treatment options.

### Example 10

### Astrocyte Cell Volume Measurements

Cortical astrocyte cultures were made from P1 Sprague-Dawley rat pups. Heterozygotes of the Cx43 knockout line were obtained from The Jackson Laboratory (Lin et al., “Connexin Mediates Gap Junction-Independent Resistance to Cellular Injury,” J. Neurosci. 23(2):430-441 (2003), which is hereby incorporated by reference in its entirety). Astrocytes were loaded with calcein/acetoxymethyl ester (AM) (5 μM for 30 min) and visualized by confocal microscopy (Schreiber et al., “The Cystic Fibrosis transmembrane Conductance Regulator Activates Aquaporin 3 in Airway Epithelial Cells,” J. Biol. Chem. 274(17):11811-11816 (1999), which is hereby incorporated by reference in its entirety). The fluorescence dilution technique was performed on astrocytes loaded with fura-2/AM (5 μM for 30 min). The volume of astrocytes in suspension was analyzed with a Coulter counter.

### Example 11

### Glutamate Measurements, Slice Preparation, and Electrophysiology

An enzymatic fluorescence detection assay for monitoring glutamate was used (Bezzi et al., “Prostaglandins Stimulate Calcium-Dependent Glutamate Release in Astrocytes,” Nature 391(6664):281-285 (1998), which is hereby incorporated by reference in its entirety). For analysis by high-performance liquid chromatography (HPLC), confluent cultures were mounted in a perfusion chamber. The amino acid content was analyzed after reaction with o-phthaldialdehyde by using fluorometric detection (Shank et al., “Cerebral Metabolic Compartmentation as Revealed by Nuclear Magnetic Resonance Analysis of D-[1-13C]Glucose Metabolism,” J. Neurochem. 61(1):315-323 (1993), which is hereby incorporated by reference in its entirety). Acutely isolated cortical or hippocampus slices prepared from 14- to 18-day-old Sprague-Dawley rats were used for electrophysiological recordings (Kang et al., “Astrocyte-Mediated Potentiation of Inhibitory Synaptic Transmission,” Nat. Neurosci. 1(8):683-692 (1998), which is hereby incorporated by reference in its entirety).

### Example 12

### Receptor-Mediated Ca²⁺-Dependent Astrocytic Swelling

To test the hypothesis that a Ca²⁺ increase is associated with a transient increase in astrocytic cell volume, relative changes in cell volume were measured using three different approaches.

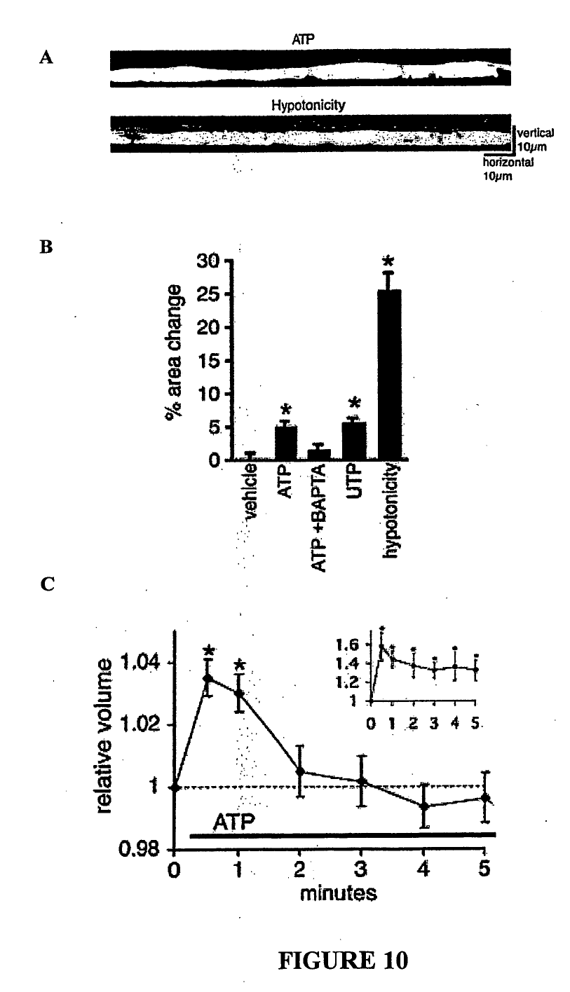

First, a confocal x-z layer scanning microscope was used Schreiber et al., “The Cystic Fibrosis Transmembrane Conductance Regulator Activates Aquaporin 3 in Airway Epithelial Cells,” J. Biol. Chem. 274(17):11811-11816 (1999), which is hereby incorporated by reference in its entirety). Vertical sections of cultured astrocytes loaded with calcein/AM (5 μM for 30 min) were constructed from repetitive x-z line scans (FIG. 10A). As several lines of work have indicated that astrocytic Ca²⁺ waves are mediated by ATP (Cotrina et al., “Connexins Regulate Calcium Signaling by Controlling ATP Release,” Proc. Natl. Acad. Sci. USA 95(26):15735-15740 (1998), which is hereby incorporated by reference in its entirety), vertical section areas of the cells were measured before and after purinergic receptor stimulation. The addition of ATP (100 μM) caused a small but significant increase of 5.19±0.66% in the vertical sectional areas, which was attenuated to 1.63±0.73% by chelation with 1,2-bis(2-aminophenoxy)ethane-N,N,N′,N′-tetraacetate (BAPTA)/AM (20 μM) (FIGS. 10A-B). Assuming that the swelling occurs only in a vertical direction in confluent cell culture, these results suggest that ATP stimulation causes a 5.2% volume increase.

Second, the use of the fluorescence-dilution method (Hanson, E., “Metabotropic Glutamate Receptor Activation Induces Astroglial Swelling,” J. Biol. Chem. 269:21955-21961 (1994), which is hereby incorporated by reference in its entirety) detected a 9.6±1.3% decrease in fluorescence-dilution emission in fura-2-loaded cultured astrocytes 1 min after ATP (100 μM) exposure compared with a 1.1±1.3% increase in emission in vehicle-controls (n=5; P<0.001, t test).

Third, exposure of astrocytes in suspension culture to ATP and subsequent assay of cell volume with a Coulter counter (Raat et al., “Measuring Volume Perturbation of Proximal Tubular Cells in Primary Culture with Three Different Techniques,” Am. J. Physiol. 271(1 Pt 1):C235-C241 (1996), which is hereby incorporated by reference in its entirety) showed a significant, reversible increase in astrocytic cell volume, averaging 3.5±0.6% at 30 sec (P<0.0003) and 3.0±0.5% at 60 sec (P<0.001); cell volume returned to prestimulation values 2 min after stimulation (FIG. 1C). In comparison, reducing external osmolarity from 314 to 214 mOsM resulted in a 58±15% increase in cell volume, which was only partly reversible (FIG. 10C inset).

Each of these independent approaches to measure cell volume demonstrated a transient increase in astrocytic cell volume in response to purinergic activation. ATP-induced swelling was modest, in the range of 3-10%, compared with the 25-60% increase in cell volume in response to hypotonicity.

### Example 13

### Pharmacological Characterization of Astrocytic Glutamate Release

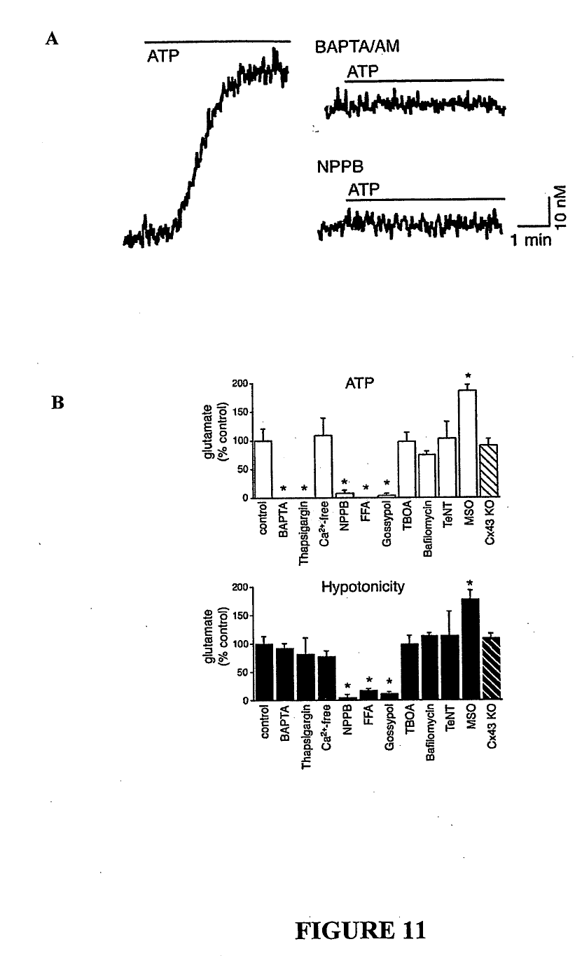

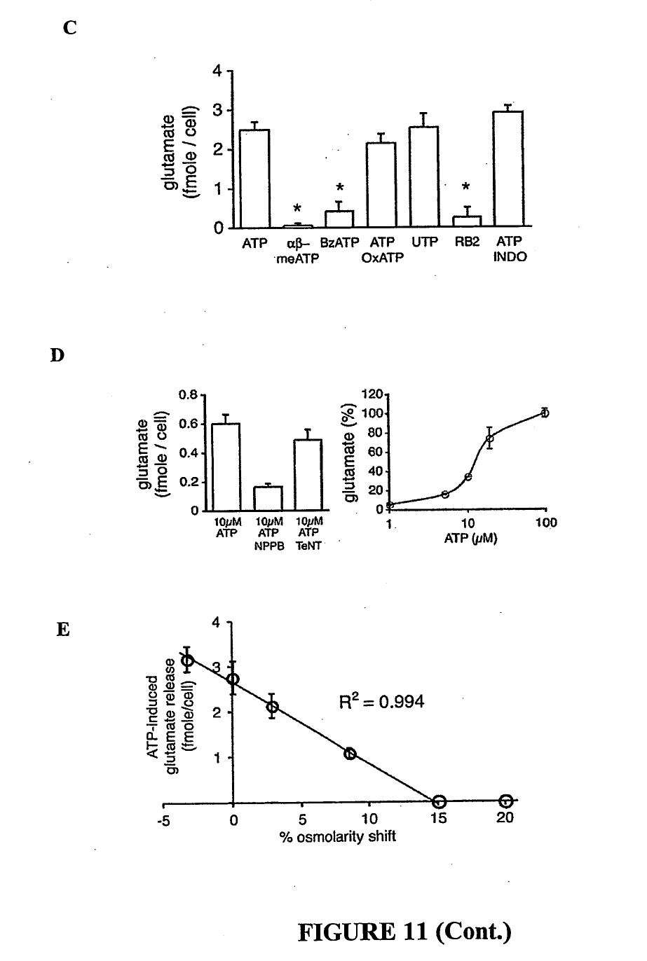

To determine whether ATP and hypotonicity induce glutamate release by the same mechanism, glutamate release from cultured rat cortical astrocytes was analyzed by using a highly sensitive enzymatic assay (Bezzi et al., “Prostaglandins Stimulate Calcium-Dependent Glutamate Release in Astrocytes,” Nature 391(6664):281-285 (1998), which is hereby incorporated by reference in its entirety). Application of 100 μM ATP resulted in the release of 2.49±0.21 fmol glutamate per cell. ATP-induced glutamate release depended on increases of cytosolic Ca²⁺, because BAPTA/AM (20 μM) for 30 min) and thapsigargin (1 μM for 10 min) attenuated the release, whereas removal of extracellular Ca²⁺ had no effect. In comparison, BAPTA and thapsigargin failed to affect glutamate release evoked by a hypoosmotic challenge (FIGS. 11A-B). Three different anion channel blockers, including 5-nitro-2-(3-phenylpropylamino)-benzoic acid (NPPB, 100 μM), flufenamic acid (FFA, 100 μM), and gossypol (10 μM), all decreased ATP- and swelling-induced glutamate release (0.22±0.14, 0.00±0.00, and 0.07±0.07 fmol per cell, respectively), whereas a noncompetitive glutamate transporter inhibitor, DL-threo-βbenzyloxyaspartic acid (TBOA, 100 μM), was without effect (FIGS. 11A-B). The application of 1 μM bafilomycin A1, an inhibitor of vesicular proton pumps for 1 h, or 2 μg/ml tetanus neurotoxin (TeNT), which inhibits exocytosis by cleaving synaptobrevin for 24 h, had no effect on glutamate release evoked by ATP or by the hypotonic challenge (FIG. 11B). However, the protocol used for application of bafilomycin A1 effectively blocked exocytosis in hippocampal slices, Within 1 hr, bafilomycin A1 reduced the frequency of inhibitory postsynaptic currents in interneurons from 2.35 per sec to 0.2 per sec (n=4; P<0.001, t test). Lastly, a 2-h application of methionine sulfoximine (MSO, 1.5 mM) (Kimelberg et al., “Swelling-Induced Release of Glutamate, Aspartate, and Taurine from Astrocyte Cultures,” J. Neurosci. 10(5):1583-1591 (1990), which is hereby incorporated by reference in its entirety), an inhibitor of glutamine synthetase that elevates the concentration of cytosolic glutamate, increased glutamate release after both ATP and hypotonicity (FIG. 11B). Together, these observations demonstrate that ATP and swelling-induced glutamate release share may characteristics but differ with regard to the dependency on cytosolic Ca²⁺:BAPTA and thapsigargin completely suppressed glutamate release evoked by ATP but had no effect on glutamate release triggered by hypotonicity.

Connexin (Cx) hemichannels have been implicated in astrocytic glutamate release after removal of extracellular divalent cations (such as Ca²⁺ and Mg²⁺) (Ye et al., “Functional Hemichannels in Astrocytes: A Novel Mechanism of Glutamate Release,” J. Neurosci. 23(9):3588-3596 (2003), which is hereby incorporated by reference in its entirety). To evaluate the role of Cx43 (the predominant member of the Cx family expressed by astrocytes), ATP-induced glutamate release from cultured astrocytes prepared from Cx43 KO mice and matched wild-type littermates was compared. ATP (100 μM) induced glutamate release of 3.02 fmol per cell from Cx43 KO astrocytes, which was 90.7% of astrocytes prepared from wild-type littermates (FIG. 11B). Therefore, astrocytes prepared from Cx43 KO mice responded similar (n 4; P=0.64, t test) to astrocytes that express Cx43. To further characterize the mechanism of ATP-induced glutamate release, the potency with which ATP agonists triggered glutamate release was evaluated (Cotrina et al., “Connexins Regulate Calcium Signaling by Controlling ATP Release,” Proc. Natl. Acad. Sci. USA 95(26):15735-15740 (1998), which is hereby incorporated by reference in its entirety). UTP (an agonist for several P2Y receptor subtypes that are expressed by astrocytes, 100 μM) triggered glutamate release with a potency that was roughly equivalent to that of ATP, and broad-spectrum P2Y receptor antagonist Reactive Blue 2 (30 μM blocked the release (FIG. 11C). By contrast, two P2X receptor agonists, αβ-meATP (100 μM) and 2′,3′-O-(4-benzoylbenzoyl)-ATP (Bz-ATP, 100 μM) were without effect (FIG. 11C). Preincubation with P2×1 and 7 receptor antagonist, oxidized ATP (OxATP; 300 μM for 1 h) did not significantly reduce ATP-induced glutamate release from cultured astrocytes. Similarly, P2×7 receptor antagonist Brilliant Blue G (BBG; 1 μM) did not reduce the glutamate release (106.4±19.1% of control). Furthermore, UTP (100 μM caused 5.75±0.57% cell swelling, similar to ATP (FIG. 10B), whereas Bz-ATP failed to induce the swelling (1.35±1.05%, n=22). These observations indicate that ATP-induced astrocytic cell swelling and glutamate release are primarily evoked by activation of P2Y receptors. Indomethacin (10 μM) did not reduce ATP-induced glutamate release, suggesting that PGE₂ production was not necessary for ATP-induced glutamate release. It was found that lowering the concentration of ATP caused a dose-dependent reduction in glutamate release (FIG. 11D). To test the idea that vesicular release contribute more significantly to glutamate release, when astrocytes were stimulated with lower and more physiological ATP concentration, we next found that NPPB (100 μM) caused 72.6±3.9% reduction after the exposure to 10 μM ATP, whereas TeNT (10 μg/ml overnight) had no effect. Thus, the relative potency of NPPB and TeNT did not significantly change when the concentration of ATP was lowered from 100 μM to 10 μM.

### Example 14

### Cell Swelling is Required for Astrocytic Ca²⁺-Dependent Glutamate Release

To determine whether cell swelling was required for Ca²⁺-dependent astrocytic glutamate release, ATP was applied simultaneously with either increasing extracellular osmolarity (inhibition of cell swelling) or decreasing osmolarity (potentiation of cell swelling). ATP-induced glutamate release from cultured astrocytes was an inverse function of extracellular osmolarity shift (regression curve: y=0.181x+2.656, R²=0.994) and completely attenuated when osmolarity was raised by 15% (FIG. 11E). This set of data confirms previous studies showing that ATP enhances swelling-induced release of excitatory amino acids released from astrocytes (Mongin et al., “ATP Regulates Anion Channel-Mediated Organic Osmolyte Release From Cultured Rat Astrocytes via Multiple Ca²⁺-Sensitive Mechanisms,” Am. J. Physiol. 288(1):C204-C213 (2005); Mongin et al., “ATP Potently Modulates Anion Channel-Mediated Excitatory Amino Acid Release from Cultured Astrocytes,” Am. J. Physiol. 283(2):C569-C578 (2002), which are hereby incorporated by reference in their entirety) and indicates that volume increase is a prerequisite for ATP-induced astrocytic glutamate release. Furthermore, because hypertonicity is known to trigger vesicular release (Sara et al., “Fast Vesicle Recycling Supports Neurotransmission During Sustained Stimulation at Hippocampal Synapses,” J. Neurosci. 22(5):1608-1617 (2002), which is hereby incorporated by reference in its entirety), the present data does not support the idea that exocytosis of glutamate containing vesicles plays a predominant role in astrocytic glutamate release.

### Example 15

### Joint Release of Amino Acid Osmolytes Evoked by Increases in Astrocytic Ca²⁺

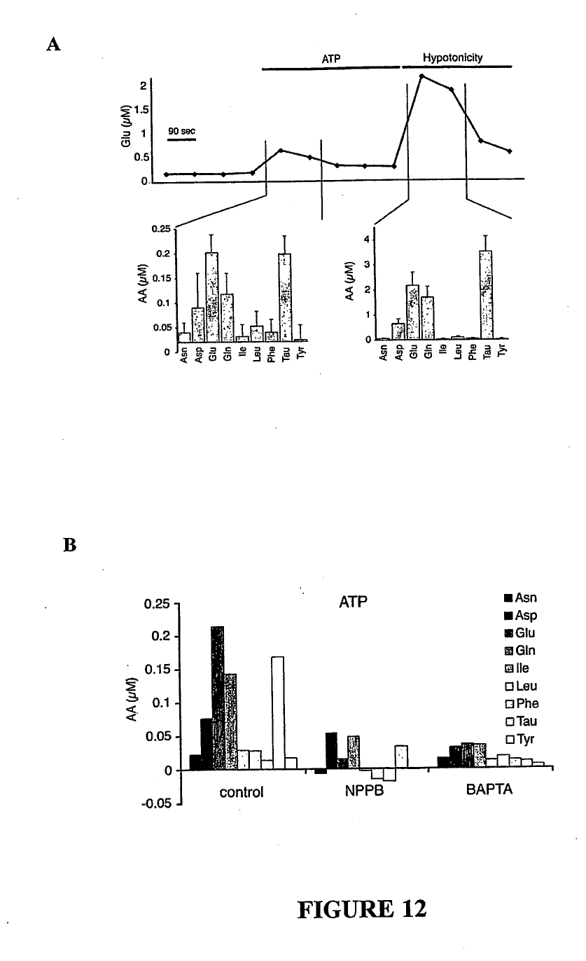

One of the characteristics of swelling-induced glutamate release is that other osmolytes, including taurine, aspartate, and glutamine, are also released in parallel (Kimelberg et al., “Swelling-Induced Release of Glutamate, Aspartate, and Taurine from Astrocyte Cultures,” J. Neurosci. 10(5):1583-1591 (1990), which is hereby incorporated by reference in its entirety). To compare the mechanism of ATP-induced, Ca²⁺-dependent astrocytic glutamate release with swelling-induced release, the extracellular concentrations of amino acids released from cultured astrocytes by using HPLC was analyzed (FIG. 12A). Interestingly, glutamate was not released in isolation, but in conjunction with taurine, aspartate, and glutamine. The profile of amino acid release in response to ATP was strikingly similar, if not identical, to swelling-induced release. In essence, purinergic stimulation induced efflux of amino acids that are regarded as osmolytes, but not of other amino acids such as asparagines, isoleucine, leucine, phenylalanine, and tyrosine (FIG. 12A). These observations strongly support the notion that volume-sensitive channels are activated during receptor-stimulated Ca²⁺ increase, resulting in efflux of cytosolic glutamate along with other amino acids. Moreover, NPPB and BAPTA/AM inhibited glutamate, as well as aspartate, glutamine, and taurine releases evoked by ATP exposure (FIG. 12B).

### Example 16

### Ca²⁺-Medicated Activation of a Channel Permeable to Glutamate

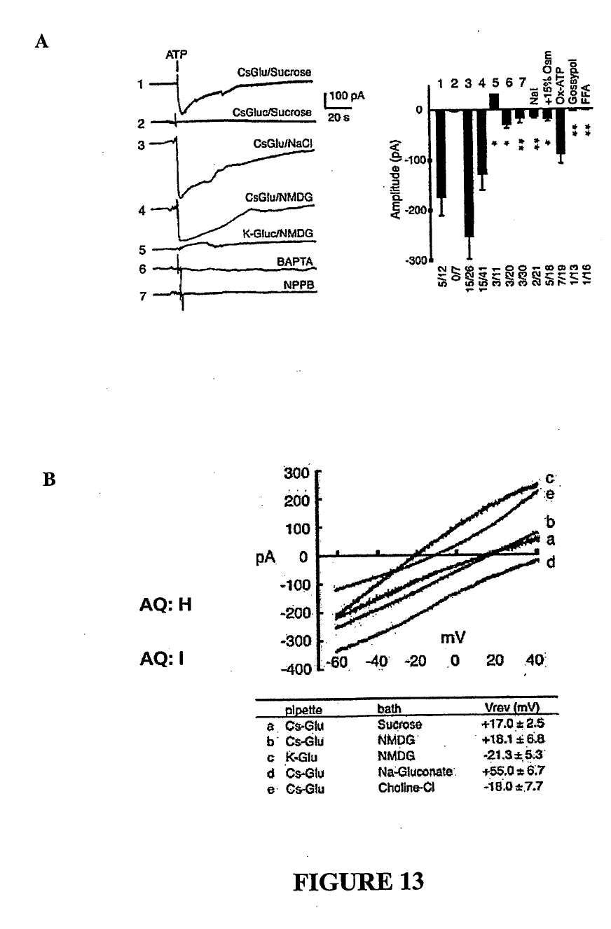

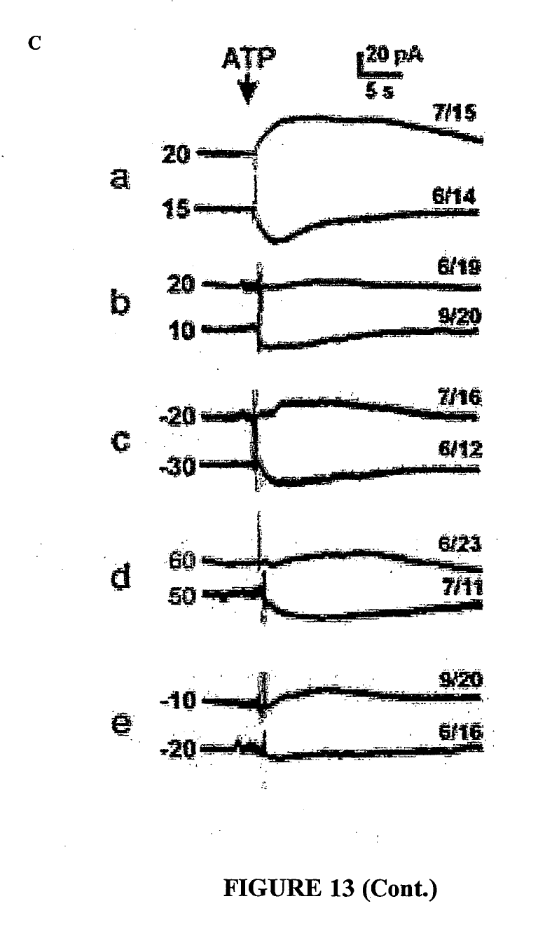

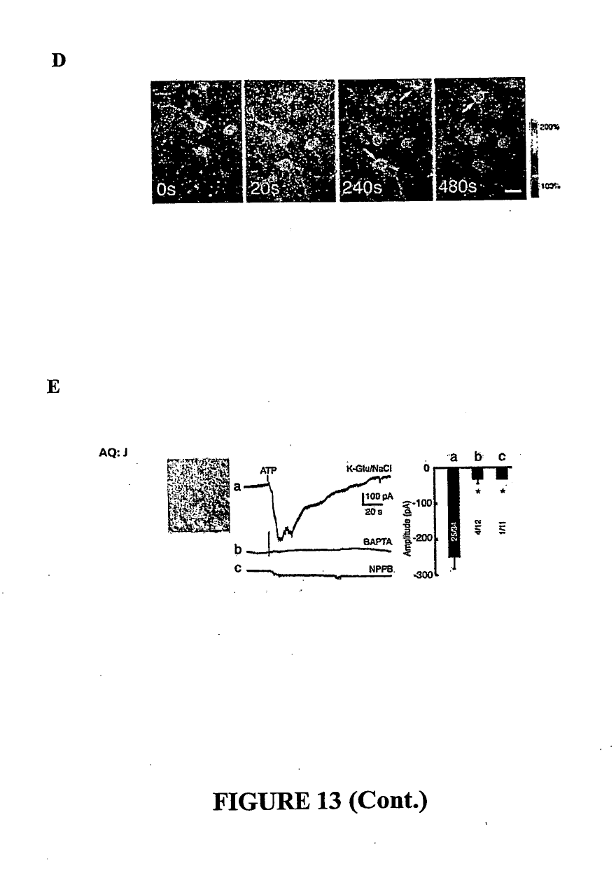

To provide direct evidence for purinergic-mediated opening of a glutamate-permeable channel, whole-cell currents in cultured astrocytes were recorded. To eliminate inward cation conductances, extracellular ions were replaced by sucrose (250 mM; osmolarity, 290 mEq) in the external solution, whereas the pipette solution contained 123 mM Cs⁺ Glutamate (Cl⁻-free). Under these ion conditions with a holding potential of −60 mV, glutamate is the only ion that can cause inward current due to its efflux. ATP (100 μM) triggered an inward current in 5 of 12 astrocytes, with average amplitude of 177±37 pA (range of 90-260 pA) (FIG. 13A-1). This observation suggests that purinergic-mediated Ca²⁺ increases are associated with the opening of a channel permeable to glutamate. Replacing glutamate with gluconate resulted in disappearance of the inward current (FIG. 13A-2), demonstrating that glutamate was the sole ion responsible for the inward current, and Cs⁺, sucrose, and gluconate⁻ were impermeable. With normal bath solution containing NaCl (126 mM) and Cs-glutamate in pipette solution, the amplitude of the current increased to 250±33 pA (range of 70-670 pA), indicating that the channel was also permeable to Na⁺ (FIG. 13A-3). When NMDG-Cl replaced NaCl in the external solution, ATP (100 μM) triggered an inward current in 15 of 41 astrocytes, with an average amplitude of 138±36 pA (range 25-290 pA), similar to sucrose substitution (FIG. 13A-4). When the pipette contained K-gluconate (glutamate-free solution), and NaCl was substituted by NMDG in the extracellular solution, no inward current was detected. Instead, ATP triggered a small outward current (FIG. 13A-5), suggesting that K⁺ also could permeate the channel. The ATP-induced current (with NaCl in the external solution) was blocked by adding 10 mM BAPTA to the pipette solution (FIG. 13A-6) in agreement with the observation that BAPTA attenuates glutamate release from astrocytic cultures (FIG. 11B). Similarly, three anion channel blockers that inhibited glutamate release, NPPB (100 μM, FIG. 13A-7), FFA (100 μM), and gossypol (10 μM), all inhibited ATP-induced current (FIG. 13A). Taken together, these observations indicate that ATP activates a glutamate-permeable channel, and that channel opening is Ca²⁺-dependent and strongly inhibited by anion channel blockers. Replacing Cl⁻ with I⁻ (Na) attenuated the inward current (FIG. 13A, NaI), in agreement with the recent observation and F potently inhibited volume recovery of cultured astrocytes (Parkerson et al., “Contribution of Chloride Channels to Volume Regulation of Cortical Astrocytes,” Am. J. Physiol. 284(6):C1460-C1467 (2003), which is hereby incorporated by reference in its entirety). Indeed, increasing the osmolarity of the bath solution by 15% decreased the amplitude of the ATP-induced current (FIG. 4A, +15% Osm). Preincubation with OxATP (300 μM for 1 h), had no significant effect on the frequency or amplitude of the ATP-induced current, suggesting that P2×7 does not play a significant role in the Ca²⁺-dependent glutamate release (FIG. 13A, Ox-ATP).

### Example 17

### Ion Permeability of the ATP-Activated Channel

Because ATP-activated glutamate-permeable channel also exhibited permeability to Na⁺ and K⁺, characterization of the ion permeability of the channel was performed. Reversal potentials under different ionic conditions were measured. Ramp commands before and after the application of ATP was first applied. The net I-V current was obtained by subtracting the I-V current before ATP application from the I-V current after ATP application (FIG. 13B). This step was taken to eliminate the large leak current of cultured astrocytes. Because subtraction of the leak currents might interfere with the measurement of reversal potential in the ramp experiments, an alternative approach to confirm the reversal potential of the ATP-induced current was used. For each ion substitution condition, two different holding potentials were used, one below and one above the reversal potential that was obtained from the ramp experiments. Astrocytes were patched in the voltage-clamp configuration, and once a stable baseline was obtained, the cells were exposed to ATP. The currents reversed between the test holding potentials (FIG. 13C), confirming the reversal potential values from the ramp experiments.

With 123 mM Cs-glutamate in the pipette and sucrose outside, the reversal potential of the ATP-induced current was +17.0±2.5 mV (FIG. 4 Ba and Ca), indicating that glutamate indeed permeated the channel. With 100 mM Cs⁺-glutamate/23 mM Cs⁺-gluconate in the pipette and NMDG outside, the reversal potential of the ATP-induced current was +18.1±6.8 mV (FIGS. 13B-b and 13C-b). Together with the observation that no current was recorded in response to ATP, when the pipette contained Cs-gluconate and extracellular NaCl was replaced by sucrose (FIG. 13A-2), this set of information indicates that gluconate and NMDG both are impermeable. When Cs⁺ was replaced by K⁺ (100 mM K⁺ glutamate/23 mM K⁺-gluconate in the pipette and NMDG outside), the reversal potential shifted leftward to −21.3±5.3 mV (FIGS. 13B-c and 13C-c), suggesting that the channel is permeable to K⁺. When NMDG was replaced by Na-gluconate, the reversal potential shifted rightward to +55±6.7 mV (FIGS. 13B-d and 13C-d), indicating that the channel is permeable to Na⁺. Of note, the tail of ramp command was 40 mV, and the reversal potential for Na⁺-gluconate was therefore calculated by extrapolation. Last, when choline chloride replaced NMDG (100 mM Cs⁺-glutamate/23 mM Cs⁺ gluconate in the pipette and 126 mM choline chloride outside), the reversal potential shifted leftward to −18.0±7.7 mV (FIGS. 13B-e and 13C-e), as compared with recordings made in the presence of Cs⁺-glutamate/sucrose or Cs⁺-glutamate/NMDG (FIGS. 13B and 13C-a-b), suggesting that the channel is also permeable to Cl⁻.

Together, these observations on ion permeability are consistent with the recordings in FIG. 13A and add further strength to the conclusion that ATP activates glutamate-permeable channels.

### Example 18

### ATP Activates Glutamate-Permeable Channel in Astrocytes in Acute Slices

To determine whether stimulation of ATP is associated with a transient increase in astrocytic Ca²⁺ concentration and activation of glutamate-permeable channels in intact tissue, ATP-induced activation of astrocytes in situ was next characterized by using two-photon microscopy and whole-cell current-clamp approach. Astrocytes in hippocampal slices were loaded with Ca²⁺ indicator dye, Fluo-4 am (Kang et al., “Astrocyte-Mediated Potentiation of Inhibitory Synaptic Transmission,” Nat. Neurosci. 1(8):683-692 (1998); Kang et al., “Imaging Astrocytes in Acute Brain Slices,” Plainview, N.Y.: Cold Spring Harbor Lab. Press (1999), which are hereby incorporated by reference in their entirety). Application of ATP (1001) evoked a 131±13% increase in the fluo-4 signal over baseline that lasted an average of 8.7±1.4 sec in the vast majority of cells (>95%, n=250) (FIG. 13D). Thus, ATP potently increased astrocytic Ca²⁺ in acute slices, similar to previous observations in cultured astrocytes (Cotrina et al., “Connexins Regulate Calcium Signaling by Controlling ATP Release,” Proc. Natl. Acad. Sci. USA 95(26):15735-15740 (1998), which is hereby incorporated by reference in its entirety). In the presence of 50 mM glutamate in the pipette solution, bath application of ATP (100 μM) induced an inward current in 25 of 34 cells (254±31 pA, range: 53-560) (FIG. 13E-a). Similar to the observations in cultured astrocytes, the ATP-induced inward current was attenuated by either 10 mM intracellular BAPTA (FIG. 13D-b) or 100 μM NPPB (FIG. 13E-c) in bath solution. BBG (1 μM) did not affect the amplitude of the inward current (106.8±22.0%; n=14; P=0.8, t test).

The main observation is that receptor-mediated astrocytic Ca²⁺ increases are associated with transient cell swelling, resulting in the activation of volume-sensitive channels and the release of cytosolic glutamate. This demonstrates that glutamate release is intimately linked to dynamic changes in astrocytic cell volume and activation of VSC. Another important observation is that cytosolic glutamate can be released in a regulated, Ca²⁺-dependent manner and, therefore, constitute a potential transmitter pool.

Direct evidence for channel-mediated efflux of glutamate was obtained by whole-cell recordings of cultured astrocytes. ATP activated a glutamate-permeable channel. The property of channel opening closely mimicked the characteristics of astrocytic glutamate release. BAPTA, NPPB, FFA, and glossypol potently inhibited both channel activation and glutamate release. Importantly, increasing both osmolarity by 15% strongly inhibited channel activation and eliminated glutamate release (FIGS. 11 and 13). The role of VSC in receptor-mediated glutamate release was illustrated by the striking similarity of the profiles of amino acids released between the receptor stimulation and the hypotonic activation of VSC (FIG. 12). Both stimulation paradigms were associated with the selective release of amino acid osmolytes, including aspartate, glutamate, glutamine, and taurine, whereas other amino acids, such as leucine, phenylalanine, and tyrosine, were not released (FIG. 12). It has previously been demonstrated that ATP potentiates hypotonicity-induced release of amino acid osmolytes (Mongin et al., “ATP Regulates Anion Channel-Mediated Organic Osmolyte Release From Cultured Rat Astrocytes via Multiple Ca²⁺-Sensitive Mechanisms, “Am. J. Physiol. 288(1):C204-C213 (2005); Mongin et al., “ATP Potently Modulates Anion Channel-Mediated Excitatory Amino Acid Release from Cultured Astrocytes,” Am. J. Physiol. 283(2):C569-C578 (2002), which are hereby incorporated by reference in their entirety), but direct evidence for receptor-mediated opening of a channel permeable to glutamate has been lacking. Taken together, these observations indicate that astrocytes release glutamate through a regulated pathway that requires mobilization of intracellular Ca²⁺ stores and activation of volume sensitive glutamate-permeable channels.

Four other possible mechanisms of glutamate release to explain the data were considered.

First, opening of Ca²⁺-activated Cl⁻ channels may provide a pathway for glutamate efflux. However, the inner pore diameter of Ca²⁺-activated Cl⁻ channels may not be large enough to allow permeation of glutamate (6.5×10.8 Å), because diphenylamine-2-carboxylic acid (DPC, 6.0×9.4 Å) failed to permeate (Qu et al., “Functional Geometry of the Permeation Pathway of Ca²⁺-Activated Cl-Channels Inferred From Analysis of Voltage-Dependent Block,” J. Biol. Chem. 276(21):18423-18429 (2001), which is hereby incorporated by reference in its entirety). Also, the dependence of astrocytic glutamate release upon medium osmolarity (FIG. 11E) does not support the role of Ca²⁺-activated Cl⁻ channels in efflux of glutamate. However, ATP may trigger opening of several types of channels, which may include both glutamate permeable and impermeable channels.

Second, P2×7 receptor-gated channels have been implicated in Ca²⁺-independent efflux of glutamate from astrocytes (Duan et al., “P2X7 Receptor-Mediated Release of Excitatory Amino Acids From Astrocytes,” J. Neurosci. 23(4):1320-1328 (2003), which is hereby incorporated by reference in its entirety). The lack of action of BzATP and OxATP lend no support to a significant contribution of P2×7 receptors in Ca²⁺-dependent glutamate release (FIGS. 11 and 13). Also, P2×7-linked channels are characterized by their cation selectivity and are not gated by cytosolic Ca²⁺.

Third, Ransom and coworkers (Ye et al., “Functional Hemichannels in Astrocytes: A Novel Mechanism of Glutamate Release,” J. Neurosci. 23(9):3588-3596 (2003), which is hereby incorporated by reference in its entirety) have recently reported the removal of divalent cations that open Cs-hemichannels, resulting in efflux of cytosolic glutamate. It was confirmed that removal of both extracellular divalent cations Mg²⁺ and Ca²⁺ resulted in sustained basal release but failed to potentiate ATP-induced glutamate release. Furthermore, astrocytes prepared from Cx43 KO and wild-type mice released comparable amount of glutamate, lending no support for the idea that Cs-hemichannels play a role in Ca²⁺-dependent glutamate release from astrocytes. This observation does not exclude that Cx-hemichannel may play important roles in glutamate release in pathological conditions, including ischemia and epilepsy (Ye et al., “Functional Hemichannels in Astrocytes: A Novel Mechanism of Glutamate Release,” J. Neurosci. 23(9):3588-3596 (2003); Tian et al., “An Astrocytic Basis of Epilepsy,” Nat. Med. 11(9):973-981 (2005), which are hereby incorporated by reference in their entirety).

Fourth, Ca²⁺-dependent exocytosis of glutamate from cultured astrocytes has been demonstrated by several groups (Montana et al., “Vesicular Glutamate Transporter-Dependent Glutamate Release From Astrocytes,” J. Neurosci. 24(12):2633-2642 (2004); Bezzi et al., “Astrocytes Contain a Vesicular Compartment That is Competent for Regulated Exocytosis of Glutamate,” Nat. Neurosci. 7(6):613-620 (2004); Kreft et al., “Properties of Ca(2+)-Dependent Exocytosis in Cultured Astrocytes,” Glia 46(4):437-445 (2004); Zhang et al., “Fusion-Related Release of Glutamate from Astrocytes,” J. Biol. Chem. 279(13):12724-12733 (2004), which are hereby incorporated by reference in their entirety). Although the present observations do not directly address the role of exocytosis in glutamate release, a number of the observations are not consistent with exocytosis constituting the primary pathway of astrocytic glutamate release. First, several anion channel blockers attenuated Ca²⁺-dependent glutamate release (FIG. 11B). Second, astrocytes did not release glutamate in isolation, but in conjunction with the release of other amino acids, osmolytes, including aspartate, taurine, and glutamine (FIG. 12). VGLUT1/2 are highly specific for glutamate and do not transport other amino acid osmolytes. Thus, the joint release of aspartate, glutamate, glutamine, and taurine indicates that channel-mediated efflux play a predominant role in Ca²⁺-mediated glutamate release. Culturing can induce astrocytes to express proteins that in situ are neuron specific. For example, synaptic vesicular protein 2 is abundantly expressed by cultured astrocytes but not by astrocytes in intact brain (Wilhelm et al., “Localization of SNARE Proteins and Secretory Organelle Proteins in Astrocytes In vitro and In situ,” Neurosci. Res. 48(3):249-257 (2004), which is hereby incorporated by reference in its entirety). In this regard, it is important to note that the ATP-induced inward current was of a similar magnitude, ≈250 pA, in astrocytes in slices and in cultures in the presence of extracellular Na⁺ (FIG. 13). Third, hyperosmotic solutions are known to trigger vesicle fusion (Pyle et al., “Rapid Reuse of Readily Releasable Pool Vesicles at Hippocampal Synapses,” Neuron 28(1):221-231 (2000), which is hereby incorporated by reference in its entirety), yet hyperosmotic solutions inhibited the glutamate release. In fact, glutamate release was an inverse function of osmolarity and was completely blocked by a 15% increase in medium osmolarity in agreement with previous reports (Mongin et al., “ATP Potently Modulates Anion Channel-Mediated Excitatory Amino Acid Release from Cultured Astrocytes,” Am. J. Physiol. 283(2):C569-C578 (2002), which is hereby incorporated by reference in its entirety) (FIG. 11E). Finally, another important argument against exocytosis as the principal pathway of glutamate release is the large quantity of the amino acid, which is released by cultured astrocytes in response to receptor activation. ATP and PGE₂ stimulation trigger glutamate release in the order of 1 nM/mg protein or ≈3 fmol of glutamate released per astrocyte (ref. 13 and FIG. 11). VGLUT1/2 expressing vesicles in astrocytes have a diameter of 30 nm (Bezzi et al., “Astrocytes Contain a Vesicular Compartment That is Competent for Regulated Exocytosis of Glutamate,” Nat. Neurosci. 7(6):613-620 (2004), which is hereby incorporated by reference in its entirety), and assuming that the concentration of glutamate is similar to synaptic vesicles (10-100 mM glutamate), each astrocyte must release 10⁵ to 10⁶ vesicles to account for the release observed (Glavinovic, M. I., “Monte Carlo Simulation of Vesicular Release, Spatiotemporal Distribution of Glutamate in Synaptic Cleft and Generation of Postsynaptic Currents,” Pfügers Arch. 437(3):462-470 (1999), which is hereby incorporated by reference in its entirety). To the contrary, TIR-FM imaging of membrane fusions of acridine orange-filled vesicles detected a total of 120 exocytotic events per astrocyte (Bezzi et al., “Astrocytes Contain a Vesicular Compartment That is Competent for Regulated Exocytosis of Glutamate,” Nat. Neurosci. 7(6):613-620 (2004), which is hereby incorporated by reference in its entirety).

The finding that astrocytes release glutamate by a regulated pathway that is sensitive to several anion channel inhibits offers an opportunity to manipulate synaptic transmission both in normal physiology and in conditions that involve the pathological activation of astrocytes, including neurodegenerative diseases. Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating or preventing epileptic seizures in a subject, said method comprising:
administering an agent which interferes with glutamate, aspartate, and/or ATP release from astrocytes to the subject under conditions effective to treat or prevent epilepetic seizures.

2. The method according to claim 1, wherein said method prevents epileptic seizures.

3. The method according to claim 1, wherein said method treats epileptic seizures.

4. The method according to claim 1, wherein said method reduces incidence of epileptic seizures.

5. The method according to claim 1, wherein said method reduces spread of epileptic seizures.

6. The method according to claim 1, wherein the agent does not suppress neural transmission.

7. The method according to claim 1, wherein the agent interferes with glutamate release from astrocytes.

8. The method according to claim 7, wherein the agent is a compound from Tables 1, 2, 3, 4, 5, or 6.

9. The method according to claim 1, wherein the agent interferes with aspartate release from astrocytes.

10. The method according to claim 9, wherein the agent is a compound from Tables 1, 2, 3, 4, 5, or 6.

11. The method according to claim 1, wherein the agent interferes with ATP release from astrocytes.

12. The method according to claim 11, wherein the agent is a compound from Tables 1, 2, 3, 4, 5, or 6.

13. A method of inhibiting hypersynchronous burst activity of a large group of neurons, said method comprising:
administering an agent which interferes with glutamate, aspartate, and/or ATP release from astrocytes to the group of neurons under conditions effective to inhibit hypersynchronous burst activity.

14. The method according to claim 13, wherein said method is carried out in vivo.

15. The method according to claim 13, wherein said method is carried out in vitro.

16. The method according to claim 13 wherein the agent interferes with glutamate release from astrocytes.

17. The method according to claim 16, wherein the agent is a compound from Tables 1, 2, 3, 4, 5, or 6.

18. The method according to claim 13, wherein the agent interferes with aspartate release from astrocytes.

19. The method according to claim 18, wherein the agent is a compound from Tables 1, 2, 3, 4, 5, or 6.

20. The method according to claim 13, wherein the agent interferes with ATP release from astrocytes.

21. The method according to claim 20, wherein the agent is a compound from Tables 1, 2, 3, 4, 5, or 6.

22. A method of identifying agents suitable for treating or preventing epileptic seizures, said method comprising:
contacting astrocytes with one or more candidate compounds;
evaluating the astrocytes for glutamate, aspartate, and/or ATP release; and
identifying the candidate compounds which interfere with glutamate, aspartate, and/or ATP release as agents potentially suitable for treating or preventing epileptic seizures.

23. The method according to claim 22, wherein said evaluating comprises:
detecting calcium release.

24. The method according to claim 22 wherein the astrocytes are evaluated for glutamate release.

25. The method according to claim 22 wherein the astrocytes are evaluated for aspartate release.

26. The method according to claim 22 wherein the astrocytes are evaluated for ATP release.
